(19) United States
(12) Patent Application Publication
(10) Pub. No.: US 2026/0294449 A1
(43) Pub. Date: Oct. 1, 2026

(54) METATARSAL OSTEOTOMY SYSTEMS AND TECHNIQUES WITH SCREW FIXATION

(71) Applicant: Treace Medical Concepts, Inc., Ponte Vedra, FL (US)

(72) Inventors: Jonathan M. Dewey, Ponte Vedra, FL (US); Caitlin DiGeorgio, Jacksonville, FL (US); Sean F. Scanlan, Jacksonville, FL (US); Adriaan Kuyler, Ponte Vedra, FL (US); Oliver Schipper, Arlington, VA (US)

(21) Appl. No.: 19/629,898

(22) Filed: Mar. 26, 2026

Related U.S. Application Data
(60) Provisional application No. 63/778,198, filed on Mar. 26, 2025.
(60) Provisional application No. 63/988,199, filed on Feb. 22, 2026.

(51) Int. Cl. A61B 17/17; A61B 17/56

(52) U.S. Cl. CPC A61B 17/1775; A61B 2017/565

(57) ABSTRACT

Systems and techniques for performing a minimally invasive metatarsal correction procedure, such as a bunion correction procedure, can utilize an instrument having a body, an intramedullary insertion body coupled to the body, a transverse-plane bone positioning device coupled to the body, and a screw targeting guide. The intramedullary insertion body and a transverse-plane bone positioning device can apply oppositely directed medial and lateral forces to controllably reposition a proximal bone portion relative to a distal bone portion. The screw targeting guide can be operatively connected to the body and have an opening defining a screw insertion trajectory for guiding a screw through proximal bone portion and into the distal bone portion for permanent fixation.

### CROSS REFERENCE

This application claims priority to and the benefit of United States Provisional Patent Application No. 63/778,198, filed Mar. 26, 2025 and United States Provisional Patent Application No. 63/988,199, filed Feb. 22, 2026. The entire contents of each of these applications are incorporated herein by reference.

### TECHNICAL FIELD

This disclosure generally relates to osteotomy devices, systems, and techniques, and more particularly, to devices, systems, and techniques for performing a percutaneous osteotomy, bone realignment, and bone fixation.

### BACKGROUND

Bones within the human body, such as bones in the foot, may be anatomically misaligned. For example, one common type of bone deformity is hallux valgus, which is a progressive foot deformity in which the first metatarsophalangeal joint is affected and is often accompanied by significant functional disability and foot pain. The metatarsophalangeal joint is laterally deviated, resulting in an abduction of the first metatarsal while the phalanges adduct. This often leads to development of soft tissue and a bony prominence on the medial side of the foot, which is called a bunion.

Surgical intervention may be used to correct a bunion deformity. A variety of different surgical procedures exist to correct bunion deformities and may involve removing the abnormal bony enlargement on the first metatarsal and/or attempting to realign the first metatarsal relative to the adjacent metatarsal. In some applications, an osteotomy is performed that involves cutting the metatarsal into two portions and shifting the cut distal portion laterally to reduce the prominence of the bunion.

For patients, surgical intervention requires making one or more incisions through the patient's skin to access the underlying bone(s) to perform a corrective procedure. A longer incision provides the surgeon with greater access to perform the procedure. However, a longer incision results in a longer scar for the patient after healing, which can be cosmetically undesirable. For this reason, the patient may prefer a shorter incision. A percutaneous procedure can provide access through one or more comparatively small puncture incisions through the skin instead of making a larger incision. While a small puncture incision can be desirable for patients, this can be challenging for the surgeon because it limits access for performing the corrective procedure. Surgical instruments that can facilitate efficient, accurate, and reproducible clinical results when working through incision opening(s) of limited size are useful for practitioners performing osteotomy and other bone realignment techniques.

### SUMMARY

In general, this disclosure is directed to devices, systems, and techniques for performing an osteotomy on one or more bones in the foot of the patient. The described devices, systems, and techniques can be utilized to partially or fully correct an anatomical misalignment of the one or more bones. Example instruments and techniques described in the present disclosure can be used to surgically access and cut a bone into two different portions, controllably realign one bone portion relative to the other bone portion, and/or fixate a moved position of the one bone portion relative to the other bone portion using one or more percutaneously inserted screws.

In some implementations, a clinician surgically accesses a bone of a foot, such as first metatarsal of the foot. The clinician may utilize an incision guide or perform a freehand cut to cut the bone into at least two portions: a distal portion which may be referred to as a capital fragment and a proximal portion. With the bone cut into two portions, the clinician can attach a bone positioning instrument to one or both bone portions. The bone positioning instrument, which may also be referred to as a bone positioner, can be configured to controllably realign the distal bone portion relative to the proximal bone portion in one or more planes.

In some configurations, the bone positioning instrument includes an intramedullary insertion body that is insertable into the proximal bone portion. For example, the intramedullary insertion body may be in the form of an elongated member insertable into the medullary canal of the proximal bone portion. The intramedullary insertion body can be operatively coupled to a translation mechanism that a clinician can control to move the intramedullary insertion body relative to the body of the bone positioner. The clinician can engage the translation mechanism to cause the intramedullary insertion body to move medially, thereby pulling the proximal bone portion medially and/or pushing the distal bone portion laterally. This can create an offset between the cut end faces of the distal bone portion and proximal bone portion, providing a correction in the transverse plane.

In some configurations, the bone positioning instrument also includes a transverse plane bone positioning device. The transverse plane bone positioning device may be located proximally along the length of the bone positioner from where the intramedullary insertion body is positioned. The transverse plane bone positioning device can include a contact surface that can apply a laterally directed force to the proximal bone portion. This may apply a force in a direction substantially opposite from the direction of the pulling force provided by translation of the intramedullary insertion body. This can help stabilize and/or temporarily attach the bone positioner to the proximal bone portion, helping to provide a stable mechanical construct that the clinician can engage with and/or remove their hands from while remaining stably engaged with the bone portion(s).

The bone positioning instrument may additionally or alternatively include one or more features to control positioning of the distal bone portion in the frontal plane and/or sagittal plane. In some examples, the bone positioning instrument includes a frontal plane bone positioning device that is configured to engage the distal bone portion and can be used to control frontal plane realignment of the distal bone portion. The frontal plane bone positioning device can engage a pin inserted (e.g., percutaneously) into the distal bone portion and can be used to control a frontal plane position of the distal bone portion.

To fixate a moved position of the distal bone portion relative to the proximal bone portion (after moving the distal portion in one or more planes), one or screws may be inserted (e.g., percutaneously) into the proximal bone portion and the distal bone portion, across the osteotomy, to fixate the bone portions relative to each other. For example, at least two screws may be inserted through the proximal bone portion and into the distal bone portion to fixate the bone portions relative to each other. The screws can hold the distal bone portion in a moved position relative to the proximal bone portion and can remain in the body after closure of the one or more incision sites. The screws can hold the distal bone portion relative to the proximal bone portion to facilitate bone in growth and fusion of the two bone portions over time, resulting in the two bone portions being fused together and again forming a unitary bone construct.

To facilitate accurate and repeatable installation of the one or more screws during the surgical procedure, the bone positioning instrument may additionally include a screw targeting functionality thereby providing a combined positioning and targeting instrument. For example, the instrument may include a screw targeting guide operatively connectable to the body of the instrument to which the intramedullary insertion body is connected. The screw targeting guide may be attached to the instrument throughout the surgical procedure (e.g., during one or more bone realignment steps) or may be attached to the body of the instrument after performing one or bone realignment steps. In either case, the screw targeting guide can define one or more openings that provide screw insertion trajectories along which one or more corresponding screws can be guided through the proximal bone portion and into the distal bone portion.

To accurately position the one or more screws during the surgical procedure, the instrument may include one or more openings for guiding one or more corresponding wires into the distal bone portion. The wires may be configured as indicating wires having a length sized to position a designated target location on the wire (e.g., a tip of the wire) at a location that intersects the screw insertion trajectory defined by the screw targeting guide. The clinician may insert multiple different indicating wires having different lengths to identify corresponding different target locations (e.g., different locations in a medial-to-lateral direction) across the distal bone portion. The clinician can select one of the multiple different target locations as the one the clinician desires to target the screw to intersect.

For example, the screw targeting guide may include a plurality of discrete screw targeting options that correspond to the different target locations identified by the indicating wires. The clinician may select one of the plurality of discrete target options by manipulating the screw targeting guide such that the screw insertion trajectory defined by the screw targeting guide intersects the target location identified by the indicating wire. In some implementations, the screw targeting guide is further adjustable to adjust the angle of the screw insertion trajectory relative to the proximal bone portion around the set target location.

With the screw targeting guide set at a desired position relative to the proximal bone portion and the distal bone portion for guiding one or more screws, the clinician can proceed with the procedure steps to insert the screws. In practice, the screws being inserted may have a tendency to skive or otherwise move off a target insertion trajectory during installation. Accordingly, systems and techniques according to the disclosure can be implemented to reduce or eliminate screw insertion inaccuracies, helping to ensure accurate and repeatable fixation.

In some examples, a technique utilizing the instrument involves advancing a drill through the screw targeting guide into the proximal bone portion (e.g., without penetrating the lateral cortical wall of the proximal bone portion). After withdrawing the drill from the proximal bone portion, a sleeve can be introduced into the hole formed in the proximal bone portion, helping to maintain alignment of the screw targeting guide with the hole formed in the proximal bone portion as well as helping to stabilize the interface between the instrument and proximal bone portion. With the sleeve inserted into the proximal bone portion, a wire can be advanced through the sleeve, penetrating through the lateral cortical wall of the proximal bone portion, and be advanced into the distal bone portion. With the wire so inserted, the sleeve can be removed over the wire and a cannulated screw advanced over the wire. The cannulated screw can be advanced until the tip of the screw is positioned at a desired target location in the distal bone portion and/or the proximal end of the screw is positioned at a desired location relative to the medial cortical wall of the proximal bone portion. The proximal end of the screw may include a chamfer or angulation that can be aligned with the medial cortical wall of the angled proximal bone portion, e.g., presenting a substantially flush interface between the bone and screw. Devices, systems, and techniques according to the disclosure can have a variety of additional or different features and aspects, as will be described herein.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIG. 4 is a flow diagram illustrating an example technique for performing an osteotomy and bone realignment procedure.

FIG. 5 illustrates one example bone preparation guide that may be used as part of an osteotomy procedure.

FIG. 6 illustrates an example bone preparation guide being secured to a first metatarsal.

FIGS. 7A and 7B are different views of an example bone positioning and screw targeting instrument according to the disclosure.

FIG. 7C is an illustration showing an example configuration of an intramedullary insertion body that includes an opening extending through the thickness of the body.

FIG. 7D is a cutaway view of a portion of the instrument from FIGS. 7A and 7B showing an example configuration of the intramedullary insertion body and connection with the body of the instrument.

FIG. 7E is another cutaway view of a portion of the instrument of FIGS. 7A and 7B.

FIGS. 8A and 8B are distal-to-proximal and medial-to-lateral side views, respectively of example configurations of a body defining a contact surface.

FIG. 8C is an illustration of an example configuration of a body defining first and second contact surfaces showing example fixation aperture pin insertion trajectories.

FIGS. 9A and 9B are dorsal-to-plantar views illustrating example procedure steps for inserting an intramedullary insertion body into a proximal bone portion.

FIGS. 9C-9E are dorsal-to-plantar views illustrating example procedure steps for engaging a transverse plane bone positioning device with a proximal bone portion.

FIG. 10 is a dorsal-to-plantar view illustrating an example procedure step for moving a distal bone portion relative to a proximal bone portion.

FIGS. 11A-11G are different views of an example bone positioning device that can be used with the instrument of FIGS. 7A and 7B to control repositioning of a bone portion in the frontal plane.

FIGS. 12A and 12B are dorsal-to-plantar views illustrating example procedure steps for engaging a frontal plane and/or sagittal plane bone positioning device with a distal bone portion.

FIGS. 13A-13C are different views of the instrument of FIGS. 7A and 7B showing example fixation aperture and fixation wire arrangements.

FIGS. 14A and 14B are different views of an example configuration of a screw targeting guide that can be used with the instrument of FIGS. 7A and 7B.

FIG. 14C is a dorsal-to-plantar view of the instrument with screw targeting guide attached illustrating an example procedure step and arrangement of components.

FIG. 15A as a partial cutaway of an example screw targeting guide showing an example selector that can be used by a clinician to select one of a plurality of identified target locations to be targeted.

FIG. 15B is an expanded partial cutaway of an example screw targeting guide showing an example adjustable interface.

FIGS. 15C-15E illustrate an example screw targeting guide interfacing with an instrument body different discrete selectable meeting positions.

FIGS. 16A-16C show different example discrete target locations at a distal bone portion that can be selected to be targeted by a screw targeting guide.

FIG. 17 is a dorsal-to-planar view showing an example entry point identification tool that can be engaged with a screw targeting guide to visualize a screw insertion trajectory.

FIGS. 18A-18F are different views illustrating an example configuration of an entry point identification tool that may be used according to disclosure.

FIGS. 19A-19O illustrate example procedure steps that can be used to install screw fixation using instruments and techniques according to disclosure.

FIG. 20 is a perspective view showing an example configuration of a plantar support mechanism for supporting a distal bone portion that can be engaged with an instrument.

FIGS. 21A-21D illustrate example procedure steps for engaging the plantar support mechanism of FIG. 20 with an instrument and actuating the plantar support mechanism to plantarly engage and support a distal bone portion.

FIGS. 22A and 22B illustrate an example cutting instrument that may be used to form an incision through the skin and/or soft tissue.

FIGS. 23A and 23B illustrate an example use of the cutting instrument of FIGS. 22A and 22B to form an incision through the skin and/or soft tissue by advancing the cutting instrument through an opening of an example screw targeting guide of the disclosure.

### DETAILED DESCRIPTION

This disclosure generally relates to devices, systems, and techniques for performing a bone osteotomy and realignment procedure in which a bone is cut into at least two portions and one portion is move relative to another portion. In an exemplary applications, the devices and techniques can be used during a surgical procedure performed on one or more bones, such as bones in the foot or hand, where the bones are relatively small compared to bones in other parts of the human anatomy. In one example, a procedure utilizing embodiments of the disclosure can be performed to correct metatarsal misalignment. An example of such a procedure is a bunion correction procedure where an osteotomy is performed on a first metatarsal of the foot to divide the first metatarsal into a proximal portion and a distal portion. The distal portion of the first metatarsal can be moved (e.g., laterally) relative to the proximal portion to reduce or eliminate the bony prominence of the bunion. Another example is a bunionette correction procedure (also known as a tailor's bunion procedure) performed on a fifth metatarsal of the foot to divide the fifth metatarsal into a proximal portion and a distal portion. The distal portion of the fifth metatarsal can be moved (e.g., medially) relative to the proximal portion to reduce or eliminate the bony prominence on the fifth metatarsal.

In some examples, an osteotomy procedure is performed to treat hallux valgus, which is referred to as a bunion. Hallux valgus, also referred to as hallux abducto valgus, is a complex progressive condition that is characterized by lateral deviation (valgus, abduction) of the hallux and medial deviation of the first metatarsophalangeal joint. Hallux valgus typically results in a progressive increase in the hallux abductus angle, the angle between the long axes of the first metatarsal and proximal phalanx in the transverse plane. An increase in the hallux abductus angle may tend to laterally displace the plantar aponeurosis and tendons of the intrinsic and extrinsic muscles that cross over the first metatarsophalangeal joint from the metatarsal to the hallux. Consequently, the sesamoid bones may also be displaced, e.g., laterally relative to the first metatarsophalangeal joint, resulting in subluxation of the joints between the sesamoid bones and the head of the first metatarsal. This can increase the pressure between the medial sesamoid and the crista of the first metatarsal head.

In some examples, an osteotomy procedure is performed to treat a tailor's bunion, also known as digitus quintus varus or bunionette. A bunionette is a callus and an adventitious bursa that overlies a prominent, laterally deviated fifth metatarsal head and a medially deviated fifth toe.

While devices and techniques are generally described herein in connection with the first metatarsal of the foot as part of a bunion correction procedure, the techniques and devices may be used on other bones and/or to treat other bone conditions. In various examples, the devices, systems, and/or techniques of the disclosure may be utilized on comparatively small bones in the foot such as a metatarsal (e.g., first, second, third, fourth, or fifth metatarsal), a cuneiform (e.g., medial, intermediate, lateral), a cuboid, a phalanx (e.g., proximal, intermediate, distal), and/or combinations thereof.

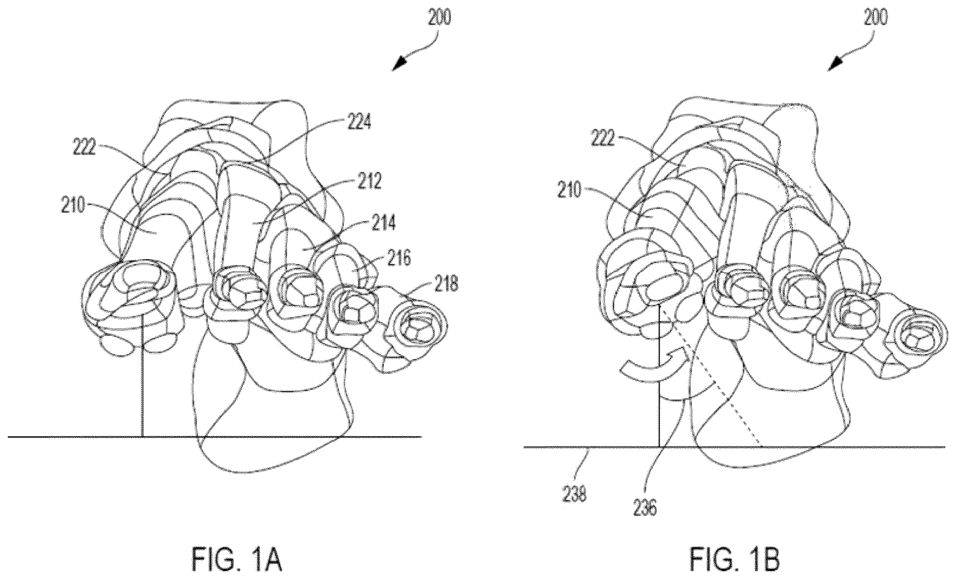

To further understand example techniques of the disclosure, the anatomy of the foot will first be described with respect to FIGS. 1-3 along with example misalignments that may occur and be corrected according to the present disclosure. A bone misalignment may be caused by hallux valgus (bunion), bunionette, a natural growth deformity, and/or other condition.

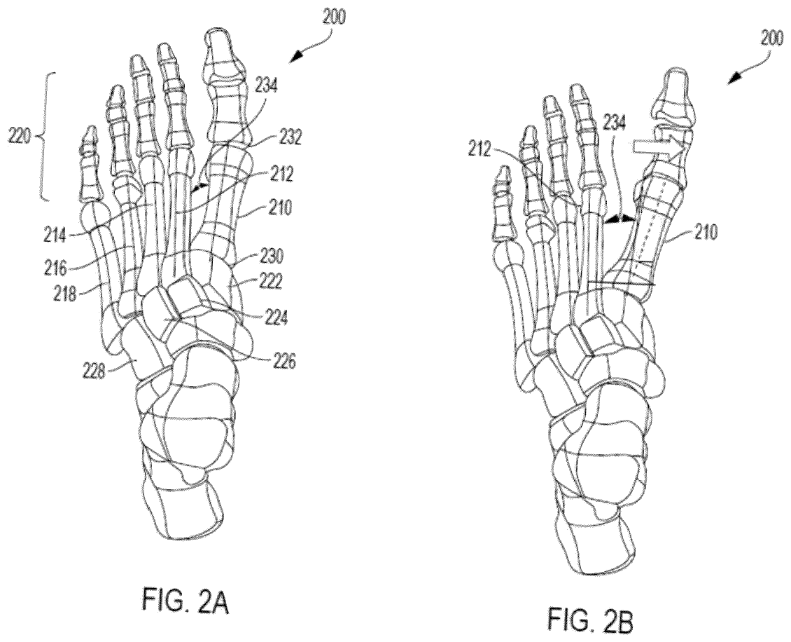

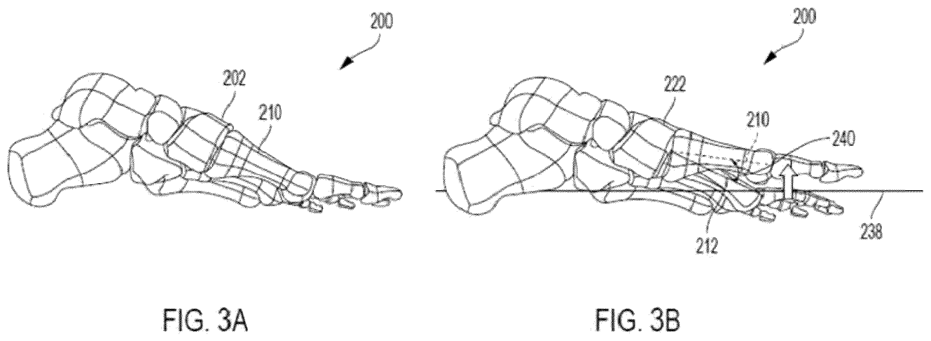

FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane misalignment in each of FIGS. 1B, 2B, and 3B is for purposes of illustration and a metatarsal may be misaligned in multiple planes that is desirably corrected. Further, a bone condition treated according to the disclosure may not present any of the example misalignments described with respect to FIGS. 1B, 2B, and 3B, and it should be appreciated that the disclosure is not limited in this respect.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. The first metatarsal 210 is connected proximally to a medial cuneiform 222, while the second metatarsal 212 is connected proximally to an intermediate cuneiform 224 and the third metatarsal is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The joint 232 between a metatarsal and respective proximal phalanx is referred to as a metatarsophalangeal (“MTP”) joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and is perpendicular to an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

Standard medical planes of reference and descriptive terminology are employed in this disclosure. A sagittal plane divides a body into right and left portions. A coronal or frontal plane divides a body into anterior and posterior portions. A transverse plane divides a body into superior and inferior portions. Anterior means toward the front of a body. Posterior means toward the back of a body. Superior or cephalad means toward the head. Inferior or caudal means toward the feet or tail. Medial means toward the midline of a body (e.g., toward a plane of bilateral symmetry of the body). Lateral means away from the midline of a body or away from a plane of bilateral symmetry of the body. Proximal means toward the trunk of the body. Distal means away from the trunk. Dorsal means toward the top of the foot or other body structure. Plantar means toward the sole of the foot or toward the bottom of the body structure.

Surgical techniques and instruments according to the disclosure can be useful to treat a misalignment of one or more bones of the foot, such as first metatarsal 210. In some applications, the technique involves surgically accessing first metatarsal 210. The clinician may utilize an incision guide to identify the location and size of the incision to be made relative to first metatarsal 210 prior to making the incision through the skin of the patient to surgically access the bone. After making the incision through the skin of the patient, the clinician may or may not attach a cutting guide, which can also be referred to as a bone preparation guide, having one or more guide surfaces configured to guide a cutting instrument. The clinician can use the cutting guide to guide the cutting instrument to cut the first metatarsal into a distal portion (which can be referred to as a capital fragment) and a residual proximal portion. Alternatively, the clinician can use a cutting instrument to cut the first metatarsal into a distal portion and a proximal portion without the aide of the cutting guide, which may be referred to as a freehand or unguided cut.

In either case, with the first metatarsal cut into two portions, the distal portion can be realigned in one or more planes relative to the proximal portion to reduce or eliminate an anatomic misalignment. For example, the distal portion can be realigned in two or more planes, or three planes relative to the proximal portion. In some examples, the distal portion is moved laterally in a transverse plane relative to the proximal portion (e.g., to reduce the bony prominence associated with the bunion deformity), the distal portion is rotated in a frontal plane relative to the proximal portion (e.g., to reposition the sesamoid bones plantarly under the distal portion), and/or the distal portion is plantar flexed or dorsiflexed in the sagittal plane. The repositioning of the distal portion can occur via the clinician's hand (e.g., grasping one or more wires inserted into the distal portion) and/or with the aid of instrumentation that applies a force in one or more planes to control repositioning of the distal portion. The clinician can install one or more screws across the osteotomy location between the distal portion and proximal portion to fixate a moved position of the distal portion relative to the proximal portion. The screws can hold the moved position of the distal portion to allow bone to form and grow between the proximal portion and moved distal portion, thereby fusing the two portions together. As will be described in greater detail below, the clinician may utilize one or more instruments, screw implants, and/or techniques according to disclosure to perform the osteotomy, bone realignment, and/or screw fixation of the realigned bone portions.

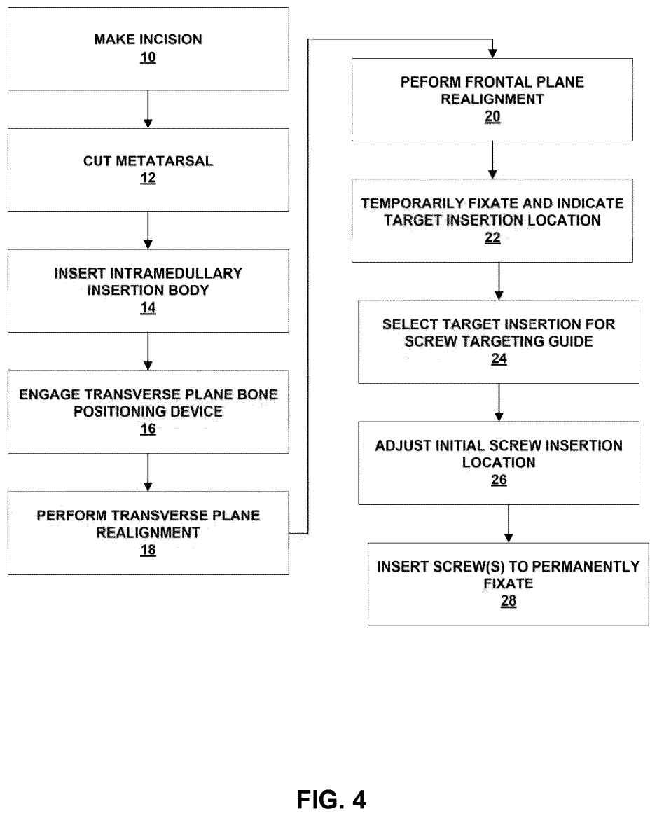

FIG. 4 is a flow diagram illustrating an example technique for performing an osteotomy, bone realignment, and fixation procedure that can use example instruments and implants according to the disclosure. The example technique will be described with respect to first metatarsal 210, although can be performed on other bones, as discussed above.

The example technique of FIG. 4 involves surgically accessing first metatarsal 210 (step 10 on FIG. 4). To surgically access the bone, the patient may be placed in a supine position on the operating room table and general anesthesia or monitored anesthesia care administered. Hemostasis can be obtained by applying thigh tourniquet or mid-calf tourniquet.

The clinician may image at least a portion of the foot 200 where first metatarsal 210 is to be cut and, correspondingly, the incision is to be made of. The clinician may take a fluoroscopic images of at least a portion of foot 200 in one or more views encompassing the region where first metatarsal 210 is to be cut. The clinician can identify the midline of first metatarsal 210 (midline between the dorsal-most surface and the planter-most surface) on the medial side of the foot based on the imaging. The clinician may position a K-wire or other radiopaque instrument along the midline while viewing the foot under imaging to identify the midline location. The clinician can also identify the metaphysis region, e.g., the first MTP joint 232 between metatarsal 210 and proximal phalanx 220. The clinician may position a K-wire or other radiopaque instrument at the MTP joint while viewing the foot under imaging to identify the MTP joint. For purposes of this disclosure, the terms wire and pin are used interchangeably and generally refer to an elongated member having a length greater than a width; the cross-sectional shape of the wire or pin may typically be circular (although other shapes can be used) and may or may not be constant across the length of the wire or pin.

The clinician may mark the medial midline location of first metatarsal 210 and/or a location of first MTP joint 230. In some examples, the clinician uses a marking source (e.g., a surgical marker pen) to indicate the midline location and/or the MTP joint. Additionally or alternatively, the clinician may percutaneously insert a wire into the midline of the foot and/or MTP joint with the wire extending out through the skin.

While first metatarsal 210 can be cut at any desired location along the length of the bone, in some examples, the location for cutting the bone is more proximal along the length of the bone than distal (e.g., the cut location may be on the proximal-most half of first metatarsal 210, such as proximal-most third of the first metatarsal, or proximal-most quarter of the proximal-most. In some examples, the cut location is set relative to a joint (e.g., MTP joint 232; TMT joint 230). For example, a target location where first metatarsal 210 is to be cut and, correspondingly, the location where an incision is to be made through the skin may be set based on the location of MTP joint 232. The clinician may identify a location offset along the midline length of first metatarsal 210 a distance distally from MTP joint 232 (e.g., as indicated by a wire extending out of the joint and/or MTP marking line). The location where first metatarsal 210 is to be cut may be within a range from 10 mm to 40 mm distal of MTP joint 232, such as 15 mm to 30 mm distal of MTP joint 232.

After identifying a target cut location, the clinician can make an incision through the skin of the patient at the target location to access the underlying bone. In different applications, the incision may be made along a medial side of first metatarsal 210, along a dorsal side of the first metatarsal, and/or along a dorsal-medial side of the first metatarsal. The incision may be comparatively small, such as having a length less than 30 mm, less than 20 mm, less than 15 mm, or less than 10 mm. The incision can be made with a cutting instrument (e.g., scalpel) and extend distal-to-proximally along the length of the metatarsal and/or dorsal-to-plantarly about the circumferential perimeter of the metatarsal.

With first metatarsal 210 exposed through the incision in the skin, the example technique of FIG. 4 includes cutting the first metatarsal to form a distal bone portion and a proximal bone portion (step 12 on FIG. 4). The clinician can use a cutting instrument to cut first metatarsal 210 into two portions. Example cutting instruments that can be used (which may also be referred to as tissue removing instruments) include, but are not limited to, a saw blade, a rotary bur, a rongeur, a reamer, an osteotome, a curette, and the like. In some examples, the clinician may use one cutting instrument (e.g., saw blade, rotary bur) to transect first metatarsal 210 into two portions and then further prepared the cut end faces of the two bone portions, e.g., by fenestrating, morselizing, and/or otherwise generating bleeding bone faces to promote fusion. In one example, the clinician uses a rotary bur to cut the first metatarsal 210 into two portions (a distal portion and a proximal portion), since a rotary bur can be effectively used through a comparatively small incision. In another example, the clinician uses a saw blade the first metatarsal 210 into two portions (a distal portion and a proximal portion).

In some examples, the clinician may cut first metatarsal 210 freehand by controlling the positioning and movement of the cutting instrument with their hand without the aid of a guide. In other examples, the clinician may cut first metatarsal 210 with the aid of a cutting guide (which can also be referred to as a bone preparation guide) having a guide surface positionable over first metatarsal 210 at the location with the bone is to be cut. When using a bone preparation guide, a cutting instrument can be inserted against a guide surface (e.g., between a slot define between two guide surfaces) to guide the cutting instrument for bone cutting. In either case, the clinician can advance the cutting instrument to cut first metatarsal 210 into two portions. The clinician can advance the cutting instrument so the cut ends of the bone portions extend at an approximately 90° angle relative to the longitudinal axis of first metatarsal 210 (e.g., by making a cut perpendicular to the longitudinal axis of the bone) or so the cut ends of the bone portions have end faces that are angled at a non-perpendicular angle with respect to the longitudinal axis of the first metatarsal 210.

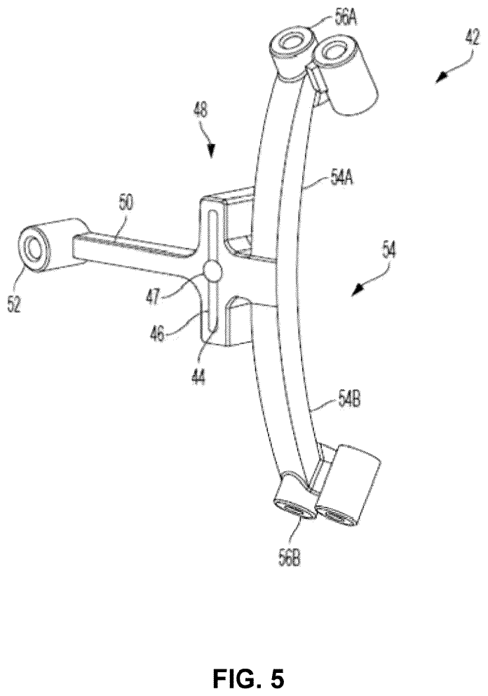

If used, a variety of different bone preparation guide configurations can be used to guide a cutting instrument. FIG. 5 illustrates one example bone preparation guide 42 that may be used as part of an osteotomy procedure. As shown, bone preparation guide 42 may define at least one guide surface 44 for guiding a cutting instrument. Guide surface 44 can be positioned over first metatarsal 210 at the location where the bone is to be cut. The clinician can guide the cutting instrument along the plane defined by guide surface 44 to control the location where first metatarsal 210 is cut. For example, the clinician may place the cutting instrument adjacent to and/or in contact with guide surface 44 and advance the cutting instrument into and through the first metatarsal 210 along the plane defined by the guide surface (e.g., by advancing the cutting instrument from the medial side of the metatarsal laterally through the lateral side of the metatarsal).

In some examples, bone preparation guide 42 defines a single guide surface 44 without opposed facing surface. In other examples, such as the example illustrated FIG. 5, bone preparation guide 42 includes a second guide surface 46, which may be referred to as a facing guide surface, defining a plane parallel to the plane defined by guide surface 44. As a result, a cutting slot can be provided between the two guide surfaces 44, 46, with the two guide surfaces bounding the extent of the cutting slot. In use, the clinician can insert the cutting instrument through the cutting slot, with the cutting slot guiding the cutting instrument to define the trajectory of the cutting instrument for cutting first metatarsal 210.

The one or more guide surfaces 44, 46 of bone preparation guide 42 may be configured to cut first metatarsal 212 transversely in the transverse plane (e.g., in a plane parallel to the frontal plane of the bone). When so configured, the cut ends of the bone portions may extend at an approximately 90° angle relative to the longitudinal axis of first metatarsal 210. In other examples, the one or more guide surfaces may be skewed in the frontal plane and/or sagittal plane such that the cut ends of the resulting bone portions have end faces that are angled at a non-perpendicular angle with respect to the longitudinal axis of the first metatarsal 210.

In some examples, such as that illustrated in FIG. 5, bone preparation guide 42 defines a pin opening 47 configured (sized and positioned) to receive K-wire 40 inserted into first metatarsal 210. For example, where K-wire 40 has a diameter within a range from 0.8 to 3.0 millimeters, such as from 0.8 mm to 2.0 mm (e.g., 1.6 mm), pin opening 47 may have a corresponding diameter sized to receive the K-wire therethrough. As illustrated, pin opening 47 is located substantially centered along the length of the one or more guide surfaces 44, 46, although in other examples may be positioned at other locations relative to the guide surfaces. Configuring bone preparation guide 42 with pin opening 47 may be useful to help position and orient the guide surfaces 44, 46 relative to the target cut location (e.g., by positioning the bone preparation over the K-wire).

In the example of FIG. 5, bone preparation guide 42 is illustrated as having a body 48 defining first guide surface 44 and second guide surface 46. Bone preparation guide 42 may define at least one pin receiving hole for pinning the bone preparation guide to the first metatarsal, such as at least one pin receiving hole positionable proximally from where first metatarsal 210 is to be cut using the bone preparation guide and at least one pin receiving hole positioned distally from where first metatarsal 210 is to be cut using the bone preparation guide. When so configured, pins can be inserted on both sides of the cut to be made using the bone preparation guide, helping to stabilize the bone preparation guide while cutting the first metatarsal and after the first metatarsal has been cut into two different portions.

In the illustrated example, bone preparation guide 42 is illustrated as including a first arm 50 extending outwardly from body 48 to define a first pin receiving hole 52 at the end of the body. First arm 50 is illustrated as extending generally perpendicularly relative to the length of guide surfaces 44, 46. As a result, when bone preparation guide 42 is positioned over first metatarsal 210 with first and second guide surfaces 44, 46 defining guide planes across the cross section of first metatarsal 210, first arm 50 may extend parallel to the longitudinal length of the first metatarsal (in a distal to proximal direction).

Bone preparation guide 42 is also illustrated as including a second arm 54 defining a second pin receiving hole 56, which is illustrated as including at least two pin receiving holes 56A and 56B. The second arm 54 may be configured to extend parallel to the longitudinal length of first metatarsal 210 (e.g., mirroring the arrangement of first arm 50). As illustrated, however, second arm 54 extends generally parallel to the plane defined by first guide surface and second guide surface 44, 46. For example, second arm 54 can define a radius of curvature extending outwardly from body 48 to position second pin receiving hole 56 at a location that is offset about the perimeter of first metatarsal 210 in the frontal plane from the first and second guide surfaces. For example, bone preparation guide 42 may include a second arm 54 having a first portion 54A configured to wrap dorsally upwardly from the medial side of the first metatarsal where the guide surfaces are to be positioned, thereby positioning pin receiving hole 56A over a dorsal-medial and/or dorsal side of the metatarsal. Bone preparation guide 42 may additionally or alternatively include a second arm 54 having a second portion 54B configured to wrap plantarly downwardly from the medial side of the first metatarsal where the guide surfaces are to be positioned, thereby positioning pin receiving hole 56B over a plantar-medial and/or plantar side of the metatarsal.

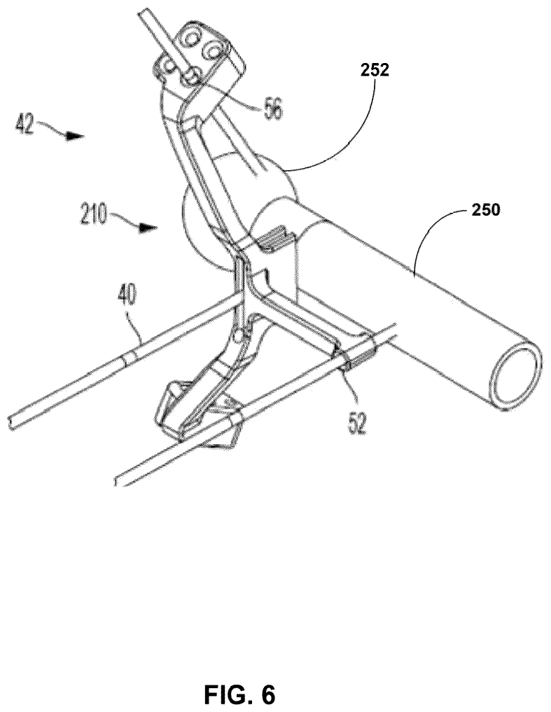

After making the incision through the skin of the patient, the clinician can insert a bone preparation guide 42 into the incision (e.g., until the cut guide abuts first metatarsal 210), optionally positioning opening 47 over a wire 40 extending out of the bone. FIG. 6 illustrates bone preparation guide 42 being secured to first metatarsal 210 with a fixation wire inserted into the metatarsal proximally of the target osteotomy location through pin receiving opening 52 and also distally of the target osteotomy location through pin receiving opening 56. Pin receiving opening 52 may be aligned with the central axis of first metatarsal 210 (e.g., using an external mark on the skin made when initially laying out the osteotomy) to ensure the resulting cut through the bone is perpendicular to the longitudinal axis of the metatarsal shaft. This can help prevent off axis movements introduced by a non-perpendicular osteotomy, helping to ensure that the cut end faces of the resulting bone portions are substantially perpendicular to the longitudinal axis of the bone (plus or minus 10 degrees or less, such as 5 degrees or less, or 3 degrees or less from absolutely perpendicular).

Once secured proximally and distally of the target osteotomy location, wire 40 introduced into metatarsal 210 at the target osteotomy location (if used) can be removed and a bone preparation instrument guided along a guide surface (e.g., through a slot bounded by two guide surfaces) to divide the first metatarsal into a proximal bone portion 250 in a distal bone portion 252. The wire inserted through pin receiving opening 52 can then be removed and bone preparation guide 42 slid off the wire extending through pin receiving opening 56. This wire inserted into distal bone portion 252 can be used subsequent during a realignment process for controlling repositioning of the distal bone portion (e.g., in the frontal plane and/or sagittal plane). In some examples, the wire(s) inserted into portion of metatarsal 210 that is subsequently formed into proximal bone portion 250 and/or distal bone portion 252 are inserted percutaneously (through the skin of the patient, such as through a stab incision) rather than through a primary incision providing access to a location where the metatarsal is cut.

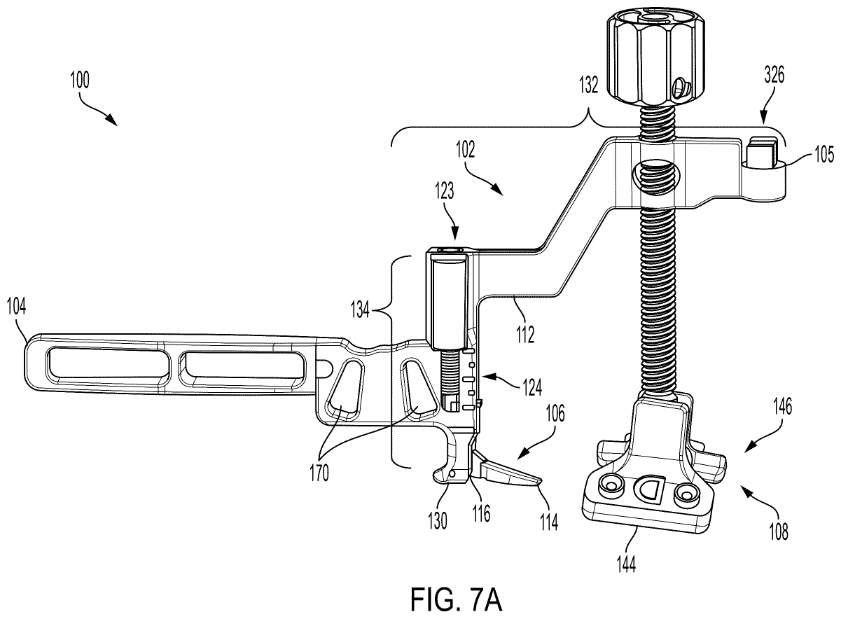

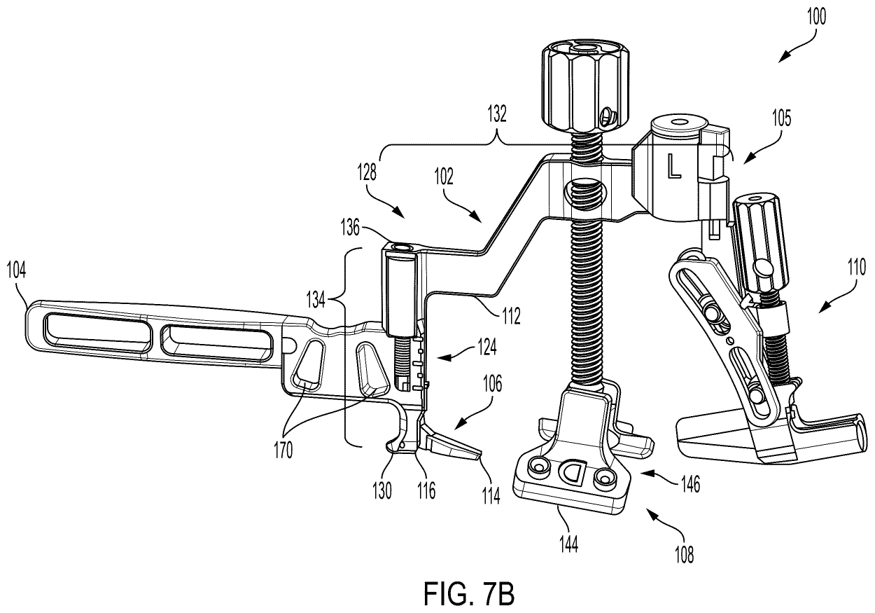

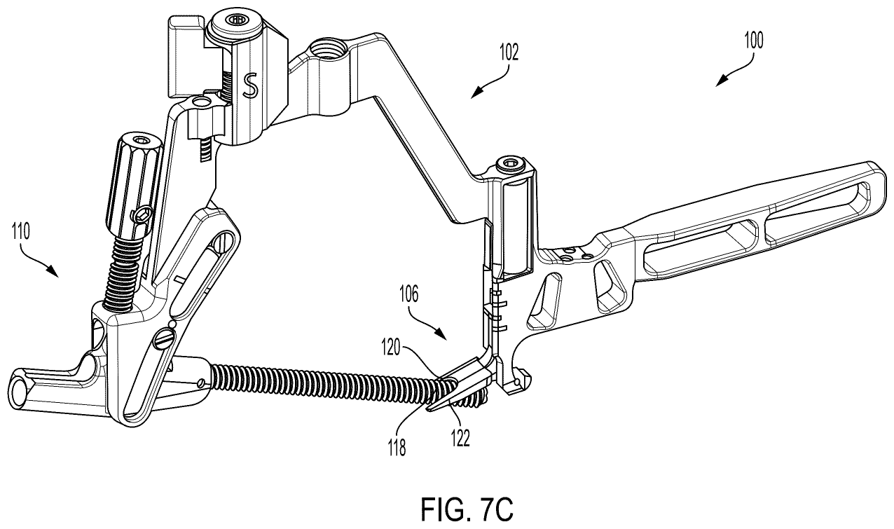

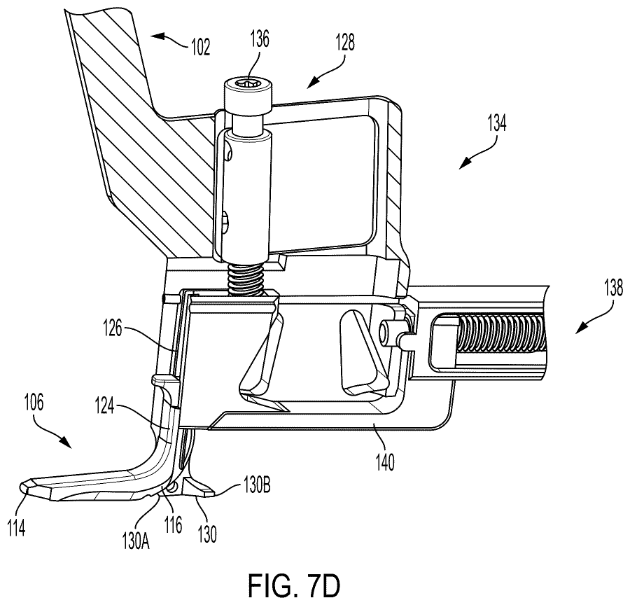

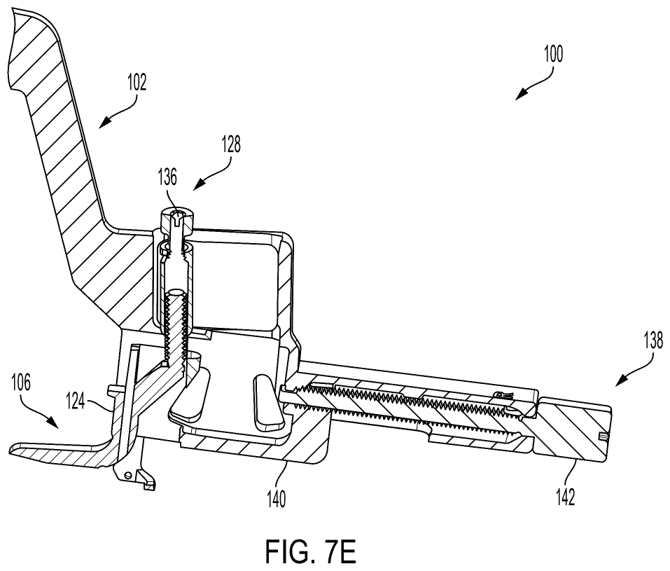

With further reference to FIG. 4, the example technique may include engaging a bone positioning and screw targeting instrument with proximal bone portion 250 and/or distal bone portion 252, including inserting an intramedullary insertion body associated with the instrument into proximal bone portion 250 (step 14 in FIG. 4). FIGS. 7A and 7B (collectively “FIG. 7”) are different views of an example bone positioning and screw targeting instrument 100 according to the disclosure. FIG. 7A is a top view of instrument 100 shown with a screw targeting guide associated with the instrument removed from the remainder of the instrument. FIG. 7B is a top view of instrument 100 shown with the screw targeting guide connected with the remainder of the instrument.

As shown in the illustrated example of FIG. 7, instrument 100 can include a body 102 that one or more functional features of the instrument are operatively connected to and that carries and holds the features for positioning relative to proximal bone portion 250 and distal bone portion 252. For example, body 102 can extend from a first end 104 to a second end 105. In use, body 102 of instrument 100 may be positioned on a medial side of first metatarsal 210 (e.g., generally parallel to and medially offset from proximal bone portion 250 and distal bone portion 252) with first end 104 position distally and second end 105 positioned proximally.

Instrument 100 can include a variety of different features configured to control positioning of distal bone portion 252 relative to proximal bone portion 250 and/or control delivery of one or more fixation screws to fixate the bone portions relative to each other. In the example of FIG. 7, instrument 100 as illustrated as including an intramedullary insertion body 106, a bone positioning device 108, and a screw targeting guide 110. In use, intramedullary insertion body 106 can be inserted into proximal bone portion 250 and engaged to apply a pulling force to the proximal bone portion. This can help offset the cut end face of proximal bone portion 250 relative to distal bone portion 252, helping to provide a transverse plane correction for the distal bone portion. In addition, bone positioning device 108 can be used to apply a force proximal of intramedullary insertion body 106, such as a laterally directed force to the sidewall of proximal bone portion 250. The force provided by bone positioning device 108 can be directed in the transverse plane, in which case bone positioning device 108 may be referred to as a transverse plane bone positioning device. The force provided by bone positioning device 108 can help stabilize the engagement of instrument 100 to proximal bone portion 250 and/or distal bone portion 252.

Screw targeting guide 110 can be used to accurately target delivery of one or more screws through proximal bone portion 250 and into distal bone portion 252, after the distal bone portion has been realigned in one or more planes relative to the proximal bone portion. For example, as will be described, screw targeting guide 110 can define one or more openings that define screw insertion trajectories that can be used for drilling screw holes through bone and/or guiding screws through the bone portions along controlled trajectories to a target insertion location.

As noted, instrument 100 is shown as including an intramedullary insertion body 106 operatively connected to body 102. Intramedullary insertion body 106 can be inserted into the medullary cannel of a bone portion (e.g., proximal bone portion 250). Intramedullary insertion body 106 can move relative to instrument body 102 to apply a positioning force between body 102 of instrument 100 and proximal bone portion 250 and/or distal bone portion 252. A clinician can translate intramedullary insertion body 106 to pull or push a bone portion engaged with the intramedullary insertion body in a direction of movement desired by the clinician. For example, during initial set up, intramedullary insertion body 106 may be positioned offset from a support surface 112 of body 102 a maximum offset distance. During subsequent use, a clinician may cause intramedullary insertion body 106 to move closer toward support surface 112 of body 102, pulling the proximal bone portion 250 away (e.g., medially outwardly) from the distal bone portion 252.

Intramedullary insertion body 106 can have a variety of different configurations. In the illustrated example, intramedullary insertion body 106 defines a length extending from a first end 114 to a second end 116. Intramedullary insertion body 106 is insertable into a medullary canal of a bone portion, e.g., by inserting first end 114 through a cut end face of a bone portion and advancing the body into the medullary canal of the bone portion. In some examples, the thickness of intramedullary insertion body 106 tapers from second end 116 to first end 114, e.g., such that the body is thicker adjacent second end 116 than at first end 114. This can provide a body with a tapered profile to help insert the body into the medullary canal.

In different implementations, intramedullary insertion body 106 may be a solid structure devoid of openings or may, instead, include one or more openings extending through the thickness of the body. For example, FIG. 7C is an illustration showing an example configuration of intramedullary insertion body 106 that includes an opening 118 extending through the thickness of the body. Opening 118 may be entirely enclosed by a remaining portion of the intramedullary insertion body 106 or may divide the intramedullary insertion body into different portions, such as a dorsal portion 120 and a plantar portion 122, with the dorsal portion positionable comparatively dorsally in the plantar portion positionable comparatively plantarly. Opening 118 may be sized to receive a drill and/or screw that can be inserted therethrough. During use, opening 118 may provide access through intramedullary insertion body 106 that a drill and/or screw guided by screw targeting guide 110 can be advanced through.

Intramedullary insertion body 106 can be operatively connected to body 102 of instrument 100 via a direct or indirect connection between the intramedullary insertion body and body 102 of instrument 100. FIG. 7D is a cutaway view of a portion of instrument 100 showing an example configuration of intramedullary insertion body 106 and connection between body 102 of instrument 100. As shown in this example, intramedullary insertion body 106 may be connected to and/or extend from a support arm 124 (optionally defining a unitary structure) operatively connected to body 102. For example, intramedullary insertion body 106 may extend at an angle relative to support arm 124 (e.g., an angle with a range from 45 degrees to 135 degrees relative to the support arm, such as approximately 90 degrees). In use, intramedullary insertion body 106 can be positioned in a medullary canal of a bone while support arm 124 resides outside of the medullary canal, e.g., such as extending transversely to a cut end face of proximal bone portion 250 into which intramedullary insertion body 106 is inserted.

Intramedullary insertion body 106 can be translatable relative to body 102 of instrument 100. For example, support arm 124 can be slidably connected to body 102 within a retention cavity via a sliding connection 126 between the support arm and body.

Instrument 100 may include an intramedullary insertion body translation mechanism 128 for controlling translational movement of the intramedullary insertion body relative to body 102. When instrument 100 is positioned on a medial side of first metatarsal 210 (e.g., substantially parallel to and medially offset from proximal bone portion 250 and distal bone portion 252) intramedullary insertion body translation mechanism 128 can be operable to move intramedullary insertion body 106 and a medial to lateral direction. For example, intramedullary insertion body translation mechanism 128 can be engaged to move intramedullary insertion body 106 medially relative to body 102 of instrument 100, thereby applying a medial-directed pulling force to proximal bone portion 250 into which the intramedullary insertion body is inserted.

Body 102 of instrument 100 can include a distal contact surface 130 positioned distally of intramedullary insertion body 106 (e.g., when instrument is engaged with proximal bone portion 250 and distal bone portion 252). Distal contact surface 130 can be configured to be positioned against distal bone portion 252 while intramedullary insertion body 106 is inserted into the medullary canal of proximal bone portion 250. Accordingly, distal contact surface 130 can bear against a proximal portion of distal bone portion 252, e.g., while intramedullary insertion body 106 provides a medial-directed pulling force pulling intramedullary insertion body 106 and proximal bone portion 250 engaged therewith medially. Distal contact surface 130 can help distal bone portion 252 remain at a substantially fixed location in the transverse plane while proximal bone portion 250 is pulled medially in the transverse plane away from the distal bone portion, thereby increasing the offset between the cut end faces of the two bone portions and helping to achieve a transverse plane alignment correction between the two bone portions.

In some applications, transverse plane correction can be produced by actively increasing the relative spacing, in a medial-to-lateral direction, between the portion of instrument 100 engaged with proximal bone portion 250 and the portion of instrument 100 bearing against distal bone portion 252. For example, while intramedullary insertion body 106 is engaged with proximal bone portion 250, intramedullary insertion body translation mechanism 128 can move intramedullary insertion body 106 medially relative to body 102 so as to apply a medial-directed pulling force to a distal end region of proximal bone portion 250. At the same time, distal contact surface 130 can bear against a proximal portion of distal bone portion 252 and thereby apply a lateral-directed pushing force to distal bone portion 252, or can maintain distal bone portion 252 against medial movement such that the relative motion between proximal bone portion 250 and distal bone portion 252 produces a lateral shift of distal bone portion 252 relative to proximal bone portion 250 in the transverse plane. In this manner, the position and alignment of distal bone portion 252 and proximal bone portion 250 can be adjusted relative to each other in the transverse plane.

It should be appreciated, however, that the relative transverse plane adjustment need not be limited to a single mode of actuation. In certain configurations, the relative displacement may be achieved primarily by moving the proximal-bone-engaging portion of instrument 100 medially relative to body 102 while the distal-bone-engaging portion remains substantially fixed relative to body 102. In other configurations, the relative displacement may be achieved primarily by moving distal contact surface 130 laterally relative to body 102 while the proximal-bone-engaging portion remains substantially fixed relative to body 102. In still further configurations, both the proximal-bone-engaging portion and the distal-bone-engaging portion may move relative to body 102, for example in opposite directions, such that the effective spacing therebetween increases and distal bone portion 252 is shifted laterally relative to proximal bone portion 250. Accordingly, instrument 100 may be configured so that relative separation between the proximal-bone-engaging feature and the distal-bone-engaging feature produces the desired transverse plane correction regardless of whether that separation is generated by medial pulling, lateral pushing, or a combination thereof.

When configured with a distal contact surface 130, the distal contact surface may be substantially coplanar with a lateral-most surface of intramedullary insertion body 106 (e.g., when the intramedullary insertion body is positioned at its full lateral displacement location away from support surface 112), such as shown in FIG. 7D. In other configurations, distal contact surface 130 may be offset (e.g., in a medial-to-lateral direction) from the lateral-most surface of intramedullary insertion body 106, when the intramedullary insertion body is positioned at its full lateral displacement location away from support surface 112. For example, distal contact surface 130 may be configured to project laterally relative to the lateral-most surface of intramedullary insertion body 106, when the intramedullary insertion body is positioned at its full lateral displacement location away from support surface 112.

Distal contact surface 130 can be positioned at a variety of different locations relative to intramedullary insertion body 106. In the illustrated configuration, distal contact surface 130 is immediately adjacent to intramedullary insertion body 106 in a distal-to-proximal direction (e.g., when the intramedullary insertion body is positioned at its full lateral displacement location away from support surface 112). When so configured, intramedullary insertion body 106 and distal contact surface 130 can, collectively, form a substantially continuous surface, e.g., with a comparatively small gap at the intersection between the intramedullary insertion body and distal contact surface, such as a gap less than 5 mm, less than 3 mm, less than 2 mm, or less than 1 mm.

In other examples, distal contact surface 130 may be separated distally from intramedullary insertion body 106 a greater distance to define a larger separation gap between the distal contact surface and intramedullary insertion body. For example, distal contact surface 130 may be separated from intramedullary insertion body 106 a distance greater than 5 mm, such as great than 10 mm, or greater than 15 mm. When so configured, distal contact surface 130 may be separated from intramedullary insertion body 106 a distance within a range from 5 mm to 50 mm, such as from 10 mm to 40 mm.

Distal contact surface 130 may be a region of instrument 100 that can contact distal bone portion 252 and help prevent the distal bone portion from moving medially as proximal bone portion 250 is moved medially in response to medial-to-lateral translation of intramedullary insertion body 106. Distal contact surface 130 can have a length extending from a proximal end 130A (FIG. 7D) to a distal end 130B which, in some examples, may be within a range from 5 mm to 75 mm, such as from 10 mm to 50 mm.

Distal contact surface 130 can engage distal bone portion 252 by directly contacting the bone portion through the incision created to separate first metatarsal 210 into two different portions. More typically, however, distal contact surface 130 may be positioned in contact with the skin covering distal bone portion 252 and can engage the distal bone portion by contacting the skin covering the distal bone portion.

While distal contact surface 130 is illustrated as being substantially planar in the example of FIG. 7, the distal contact surface can have other configurations. For example, the distal contact surface may define a concave body that wraps partially or fully about the distal bone portion 252, allowing the distal contact surface to engage the bone portion on a dorsal side, a dorsal-medial side, a plantar-medial side, and/or a plantar side in addition to or in lieu of engaging the bone portion on a medial side. Other surface shapes and configurations can also be used.

In some examples, instrument 100 can engage distal bone portion 252 using another engagement configurations in addition to or in lieu of utilizing distal contact surface 130. For example, instrument 100 can include one or more fixation apertures configured to receive a corresponding fixation pins inserted there through that can extend into distal bone portion 252. The one or more fixation pins can be used to engage distal bone portion 252, e.g., helping to prevent medial movement of the distal bone portion as the proximal bone portion 250 is moved medially, in addition to or in lieu of the counter force provided by the presence of distal contact surface 130.

In some applications such as that illustrated in FIG. 7, body 102 of instrument 100 includes multiple different body segments that join together to form body 102 (e.g., with the different body segments being form separately as substantive joined together or being formed as a unitary, integral structure). The different body segments can be arranged relative to each other so as to position different functional features of instrument 100 at desired locations. For example, body 102 of instrument 100 can include a first arm segment 132 which may have a length configured to be positioned generally parallel to the longitudinal axis of first metatarsal 210 when performing a procedure (e.g., generally parallel to the longitudinal axis of proximal bone portion 250 and distal bone portion 252, prior to realignment). Body 102 of instrument 100 can also include a second arm segment 134 which may have a length configured to be positioned generally perpendicular to the longitudinal axis of first metatarsal 210 when performing a procedure (e.g., generally perpendicular to the longitudinal axis of proximal bone portion 250 and distal bone portion 252, prior to realignment). Second arm segment 134 can extend from first arm segment 132, e.g., in a lateral direction during use, positioning the terminal end of the second arm more laterally than first arm segment 132.

In the illustrated arrangement, distal contact surface 130 is positioned on and/or defined by a terminal end of second arm segment 134 of body 102. Intramedullary insertion body 106 is configured to translate along second arm segment 134 toward and/or away from support surface 112 defined by first arm segment 132. For example, intramedullary insertion body 106 may be configured to translate within a cavity defined by second arm segment 134 along sliding connection 126 (FIG. 7D). Intramedullary insertion body 106 may be configured to translate generally parallel to the longitudinal axis of second arm segment 134 and/or in a medial-to-lateral direction (e.g., generally perpendicular to the longitudinal axis defined by first metatarsal 210).

Intramedullary insertion body translation mechanism 128 can be configured to controllably move intramedullary insertion body 106 relative to body 102 to adjust the position of proximal bone portion 250 relative to distal bone portion 252. In the illustrated arrangement, translation mechanism 128 is shown as a threaded shaft connected to intramedullary insertion body 106 on one end and connected to body 102 on an opposite end. The threaded shaft can include a drive receptacle 136 (FIG. 7D) configured to receive a driver (e.g., hexalobular driver) through which a rotational force can be applied to turn the shaft. In some examples, a knob is provided on the end of the threaded shaft through which a rotational force can be applied to control positioning of intramedullary insertion body 106. In either case, turning the threaded shaft one direction can cause the shaft to pull intramedullary insertion body 106 toward support surface 112 (e.g., medially when instrument 100 is in use). Turing the threaded shaft in the opposite direction can cause the threaded shaft to push intramedullary insertion body 106 away from support surface 112 (e.g., laterally when instrument 100 is in use). While instrument 100 illustrates intramedullary insertion body translation mechanism 128 as comprising a threaded shaft, other mechanical linkages can be used that causes movement of one feature to apply a translational force to intramedullary insertion body 106, such as a rack and pinion, a ratch connection, and/or other mechanical linkage.

In some configurations, intramedullary insertion body 106 is configured to move in a distal-to-proximal direction in addition to or in lieu of being configured to move in a medial-to-lateral direction. For example, intramedullary insertion body 106 can be configured to move generally parallel to the longitudinal axis defined by body 102 of instrument 100 in addition to or in lieu of being configured to move generally perpendicular to the longitudinal axis defined by the body.

Configuring intramedullary insertion body 106 to move in a distal-to-proximal direction can be useful to control the depth to which the intramedullary insertion body extends into proximal bone portion 252. During a procedure, for instance, the clinician may insert intramedullary insertion body 106 to its full depth (e.g., such that support arm 124 and/or second arm segment 134 are adjacent to and/or in contact with the cut end face of proximal bone portion 250). The clinician may translate intramedullary insertion body 106 distally, causing first end 114 of the intramedullary insertion body to retract towards the cut end face of proximal bone portion 250 such that the intramedullary insertion body does not extend as deeply in the bone portion. This can be useful, for example, when inserting temporary or permanent fixation (e.g., wires, screws) if intramedullary insertion body 106 is located in a position that will intersect and interfere with insertion of the fixation. Withdrawing intramedullary insertion body 106 partially but not fully out of proximal bone portion 250 may remove the intramedullary insertion body from interfering with the insertion pathway of the fixation.

Accordingly, in some applications, intramedullary insertion body 106 can be translatable relative to body 102 of instrument 100 in a second direction in addition to being translatable in a first direction via intramedullary insertion body translation mechanism 128. This can allow bidirectional translation/movement of intramedullary insertion body 106 relative to body 102 of instrument 100 and/or proximal bone portion 250.

Instrument 100 may include an intramedullary insertion body translation mechanism 138 (FIG. 7D) for controlling translational movement of the intramedullary insertion body relative to body 102 in a proximal-to-distal direction. When configured with two different translation mechanisms, intramedullary insertion body translation mechanism 128 may be referred to as a first or medial-to-lateral intramedullary insertion body translation mechanism, and intramedullary insertion body translation mechanism 138 may be referred to as a second or proximal-to-distal intramedullary insertion body translation mechanism. It should be appreciated that reference to first, second, and/or other numerical order herein is intended to distinguish one feature from another feature and does not require or imply a specific order of operation of use unless otherwise specified. Accordingly, a clinician may utilize second intramedullary insertion body translation mechanism 138 before first intramedullary insertion body translation mechanism 128, or vice versa, without departing from the scope of disclosure.

When instrument 100 is positioned on a medial side of first metatarsal 210 (e.g., substantially parallel to and medially offset from proximal bone portion 250 and distal bone portion 252) intramedullary insertion body translation mechanism 138 can be operable to move intramedullary insertion body 106 and a proximal-to-distal direction. For example, intramedullary insertion body translation mechanism 138 can be engaged to move intramedullary insertion body 106 distally relative to body 102 of instrument 100, thereby retracting the intramedullary insertion body partially out of the cut end face of proximal bone portion 250 into which the intramedullary insertion body is inserted.

In some examples, intramedullary insertion body 106 is operatively connected to body 102 of instrument 100 via a movable body 140. Movable body 140 may be retained in a receiving cavity defined by a portion of body 102 of instrument 100, e.g., forming a sliding connection between the movable body and one or more interior surfaces of the receiving cavity. Intramedullary insertion body 106 including support arm 124 may be operatively connected to movable body 140 and can move with the movable body. In some configurations, such as that illustrated, first intramedullary insertion body translation mechanism 128 is also movable with movable body 140 within a bounded range of travel. Movable body 140 may move parallel to the longitudinal axis defined by body 102 of instrument 100.

In the illustrated arrangement, intramedullary insertion body translation mechanism 138 is shown as including a threaded shaft connected to intramedullary insertion body 106 via movable body 140. In some examples, a knob is provided on the end of the threaded shaft through which a rotational force can be applied to control positioning of the movable body and, correspondingly, intramedullary insertion body 106. For example, FIG. 7E is another cutaway view of a portion of instrument 100 showing an example configuration of intramedullary insertion body 106. As shown in this example, intramedullary insertion body translation mechanism 138 includes a threaded shaft connected to an actuator 142 (e.g., a knob) through which a rotational force can be applied to turn the shaft. The threaded shaft is illustrated as extending through a portion of body 102 of instrument 100 that defines a handle that can be grasped by a clinician to manipulate instrument during use. In other examples, the threaded shaft can include a drive receptacle configured to receive a driver (e.g., hexalobular driver) through which a rotational force can be applied to turn the shaft in lieu of a knob. Further while instrument 100 illustrates intramedullary insertion body translation mechanism 138 as comprising a threaded shaft, other mechanical linkages can be used that causes movement of one feature to apply a translational force to intramedullary insertion body 106, such as a rack and pinion, a ratch connection, and/or other mechanical linkage.

First intramedullary insertion body translation mechanism 128 and/or second intramedullary insertion body translation mechanism 138 can be configured to move intramedullary insertion body 106 a distance effective to perform the functions described herein during a surgical procedure. For example, first intramedullary insertion body translation mechanism 128 can be configured to move intramedullary insertion body 106 in a medial-to-lateral direction (e.g., medially or generally perpendicular to a longitudinal axis of body 102) a distance of at least 1 mm, such as at least 2 mm, at least 3 mm, at least 4 mm, at least 5 mm, at least 6 mm, at least 7 mm, or at least 8 mm. In some cases, first intramedullary insertion body translation mechanism 128 can be configured to move intramedullary insertion body 106 in a medial-to-lateral direction (e.g., medially or generally perpendicular to a longitudinal axis of body 102) a distance within a range from 2 mm to 20 mm, such as from 5 mm to 15 mm. Second intramedullary insertion body translation mechanism 138 can be configured to move intramedullary insertion body 106 in a proximal-to-distal direction (e.g., distally or generally parallel to a longitudinal axis of body 102) a distance of at least 1 mm, such as at least 2 mm, at least 3 mm, at least 4 mm, at least 5 mm, at least 6 mm, at least 7 mm, or at least 8 mm. In some cases, second intramedullary insertion body translation mechanism 138 can be configured to move intramedullary insertion body 106 in a proximal-to-distal direction (e.g., distally or generally parallel to a longitudinal axis of body 102) a distance within a range from 2 mm to 20 mm, such as from 5 mm to 15 mm.

With further reference to FIGS. 7A and 7B, instrument 100 can includes a bone positioning device 108 that can apply a force between one or more bone portions contacted by the bone positioning device and the instrument. For example, bone positioning device 108 can define a contact surface 144 that can contact a bone portion (e.g., directly or indirectly via an overlying skin layer that the contact surface touches). For example, in the illustrated arrangement, bone positioning device 108 is illustrated as being positioned between intramedullary insertion body 106 and screw targeting guide 110. In use when instrument 100 is engaged with proximal bone portion 250 and/or distal bone portion 252, bone positioning device 108 and, more particularly, contact surface 144, can be positioned distally of screw targeting guide 110 and proximally of intramedullary insertion body 106.

In use, bone positioning device 108 can be actuated to apply a force between a bone portion underlying contact surface 144 and body 102 of instrument 100. For example, bone positioning device 108 can apply a force in the transverse plane, such as a laterally-directed force that may be substantially perpendicular to the longitudinal axis of first metatarsal 210 (e.g., the longitudinal axes of proximal bone portion 250 and/or distal bone portion 252). When configured to apply a transverse plane directed force, bone positioning device 108 may be characterized as a transverse-plane bone positioning device.

The force applied by bone positioning device 108 can be effective to help realign distal bone portion 252 relative to proximal bone portion 250. Additionally or alternatively, the force applied by bone positioning device 108 can help the instrument stably engage foot 200 during a surgical procedure. For example, bone positioning device 108 can provide a counterforce (e.g., laterally-directed pushing force) to the force provided by intramedullary insertion body translation mechanism 128 (e.g., a medially-directed pulling force). The pressure between contact surface 144 and the underlying bone portion (e.g., skin covering the bone portion) can help stabilize instrument 100 during the surgical procedure. The clinician can visualize the bone portions under imaging (e.g., fluoroscopy) with instrument 100 attached. The clinician can evaluate the relative position of distal bone portion 252 to proximal bone portion 250. The clinician can adjust the position of distal bone portion 252 relative to proximal bone portion 250 (e.g., by hand and/or through the use of instrument 100) and/or the position of one or more screw implants implant relative to one or both bone portions until desired positioning is achieved.

Instrument 100 may stably engage the foot when the interaction between the intramedullary insertion body 104, the bone positioning device 108, and the underlying anatomy provides a self-maintained engagement state. For example, opposing forces generated between the intramedullary insertion body 104 and the bone positioning device 108 can bias instrument 100 into a seated position relative to the foot such that the position and orientation of instrument 100 are maintained without continuous manual support by the clinician. In this context, stable engagement may refer to the ability of instrument 100 to remain coupled to the foot in a substantially fixed position and/or angular orientation for a period of time sufficient to permit one or more procedural steps, such as fluoroscopic evaluation, bone manipulation, trajectory assessment, implant positioning, drilling, or screw placement, while the clinician physically releases instrument 100. Such stable engagement can reduce unintended shifting of instrument 100 relative to the foot and can facilitate more consistent alignment during the procedure.

Indeed, body 146 can define contact surface 144 that is configured as a proximal pusher that functions not only to apply a transverse-plane counterforce, but also to stabilize instrument 100 relative to proximal bone portion 250 and help set the angular orientation of the instrument relative to the metatarsal in the sagittal plane. For example, when contact surface 144 engages the medial, dorsal-medial, and/or plantar-medial region of proximal bone portion 250 and is advanced laterally against the underlying anatomy, the resulting counterpressure can bias body 102 into a seated position on the foot and help maintain the longitudinal axis of the instrument in a desired orientation, such as substantially parallel to the longitudinal axis of the metatarsal shaft. In this manner, contact surface 144 may help resist rocking, tipping, or other unintended movement of instrument 100 during imaging, alignment, drilling, and/or implant placement, while also contributing to transverse plane realignment. In some configurations, the geometry of body 146, including relative sizing and positioning of contact surface 144, can further help establish a desired plantarflexion and/or dorsiflexion bias on proximal bone portion 250, thereby assisting sagittal plane positioning in addition to transverse plane stabilization and correction. Additionally or alternatively, such counterpressure-based stabilization can reduce or eliminate reliance on fixation pins to maintain instrument position, although fixation apertures and fixation wires may still be used in other embodiments.

In the illustrated arrangement, bone positioning device 108 is shown as including a knob connected via a threaded shaft to body 102 of instrument 100. Body 102 can define a threaded opening through which a threaded shaft can translate. Engaging the knob of device 108 in one direction can cause the threaded shaft to advance contact surface 144 linearly away from body 102 to push the contact surface against a bone portion against which the contact surface is engaged. Engaging the knob of device 108 in the opposite direction can cause the threaded shaft to advance linearly in an opposite direction to draw contact surface 144 linearly toward body 102. In other examples, the threaded shaft can include a drive receptacle configured to receive a driver (e.g., hexalobular driver) through which a rotational force can be applied to turn the shaft in lieu of a knob. Bone positioning device 108 can implemented using a variety of additional or different force delivery mechanisms, such as a sliding connection, rack and pinion, ratch connection, and/or other mechanical linkage that causes movement of one feature to apply a translational force to the structure defining contact surface 144 and/or a pin engaged therewith and inserted into proximal bone portion 250.

The shaft of bone positioning device 108 can be arranged a variety of different ways relative to body 102 of instrument 100. With respect to FIG. 7, body 102 of instrument 100 can define a longitudinal axis extending parallel to the length of the body. Bone positioning device 108 can also define a longitudinal axis extending parallel to the length of the shaft of the bone positioning device. The longitudinal axis of bone positioning device 108 can intersect the longitudinal axis of body 102 at an angle, which may be a 90 degree angle as illustrated in FIG. 7 or a non-90 degree angle. Angling the shaft relative to the body 102 of instrument 100 can help apply a directed force when positioning one bone portion relative to another one portion using the instrument.

Contact surface 144 of bone positioning device 108 can have a variety of different configurations. In some examples, contact surface 144 is defined by a body 146 translatable relative to a shaft operatively connected to body 102 of instrument 100 or is defined by a body 146 coupled to a shaft. For example, body 146 can be positioned in an end of the shaft of bone positioning device 108, e.g., either at a fixed orientation or adjustable orientation relative to the shaft. In some examples, contact surface 144 is defined by a comparatively small cross-sectional area feature (e.g., a pin inserted percutaneously into proximal bone portion 250 and optionally having an enlarged region pressing against the proximal bone portion 250, such as against the skin overlying the metatarsal bone portion). In other examples, body 146 defining contact surface 144 is comparatively larger to transmit the force over a larger surface area.

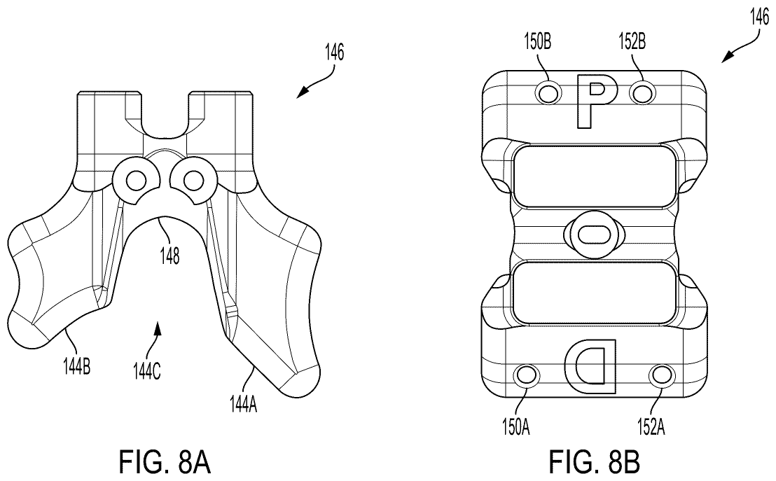

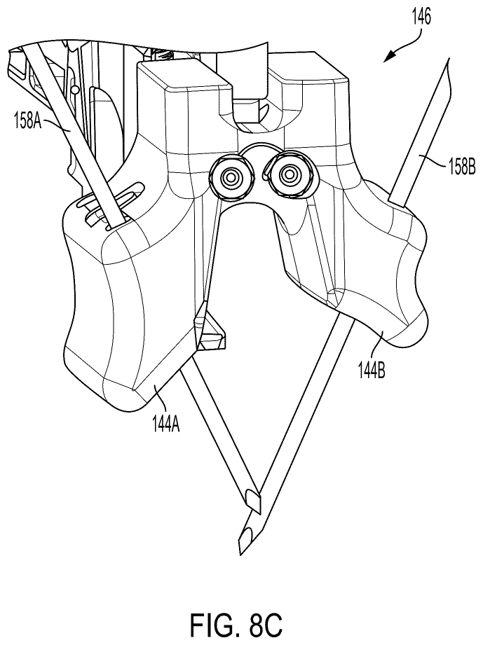

FIGS. 8A and 8B (collectively “FIG. 8”) are distal-to-proximal and medial-to-lateral side views, respectively of example configurations of body 146 defining contact surface 144 (with the direction orientations described relative to example positioning of instrument 100 during use). In some examples, contact surface 144 defined by body 146 is a cup having a concave shape configured to conform (e.g., partially wrap about) a generally cylindrical bone portion engaged with the contact surface. The contact surface 144 may be a solid, unbroken surface configured to generally distribute contact loading with a patient's tissue. In other examples, body 146 may define one or more grooves, openings, or other access features dividing contact surface 144, e.g., to allow room for insertion of wires, screws, drill bits, or other targeting implements.

In the example of FIG. 8, body 146 defines a first contact surface 144A, a second contact surface 144B, and a cutout 144C between the first contact surface and the second contact surface. First contact surface 144A can be configured to extend at least partially over a dorsal-medial and/or dorsal surface of proximal bone portion 250 during use of instrument 100. Second contact surface 144B can be configured to extend at least partially over a plantar-medial and/or plantar surface of proximal bone portion 250 during use of the instrument. Proximal bone portion 250 can be partially or fully received in cutout 144C between first contact surface 144A in second contact surface 144B. When first contact surface 144A in second contact surface 144B are in contact with proximal bone portion 250 (e.g., the skin covering the proximal bone portion), a void space may remain between a lateral wall surface 148 bounding cutout 144C and the medial side of the proximal bone portion. Configuring body 146 with cutout 144C between opposed contact surfaces 144A, 144B can be useful to provide an opening through the body through which a drill bit, screw, wire, and/or another instrument guided by screw targeting guide 110 can be advanced through proximal bone portion 250 (and optionally through the cutout of body 146) and into distal bone portion 252 without interference of the body.

In different configurations, cutout 144C may bisect and/or be substantially centered between first contact surface 144A and second contact surface 144B or may be offset closer to one contact surface than the other contact surface. Further, in various examples, first contact surface 144A and second contact surface 144B may be symmetrical (e.g., have substantially the same size and shape) or may be asymmetrical (e.g., have a different size and/or shape).

In FIG. 8, first contact surface 144A and second contact surface 144B are illustrated as being asymmetrically sized. First contact surface 144A and second contact surface 144B may be defined by sidewalls extending from a concavity or groove defining cutout 144C. One of the sidewalls can extend a different (larger) distance from the bottom of cutout 144C (e.g., defined by lateral wall surface 148) than the other sidewall, which can extend a smaller distance from the bottom of cutout. Configuring body 146 with asymmetrically extending first contact surface 144A and second contact surface 144B can be useful for applying a force that is biased in one direction instead of being applied uniformly across body 165.

In the example of FIG. 8, first contact surface 144A configured to contact the dorsal and/or dorsal-medial side of proximal bone portion 250 extends outwardly (e.g., laterally in use) a greater distance than second contact surface 144B configured to contact the plantar and/or plantar-medial side of the bone. This can apply a dorsal biased force when actuating bone positioning device 108 tending to plantar flex proximal bone portion 250. In other configurations, the second contact surface 144B configured to contact the plantar and/or plantar-medial side of the bone can extend outwardly (e.g., laterally in use) a greater distance than first contact surface 144A configured to contact the dorsal and/or dorsal-medial side of proximal bone portion 250. This can apply a plantar biased force when actuating bone positioning device 108 tending to dorsiflex proximal bone portion 250.

In some examples, bone positioning device 108 includes one or more fixation apertures for pinning contact surface 144 to proximal bone portion 250. As one example, bone positioning device 108 may include an opening or cannulation extending the length of the threaded shafted through body 146 and contact surface 144 through which a fixation wire can be inserted. Additionally or alternatively, bone positioning device 108 may include one or more fixation openings offset from the shaft of the device through which one or more corresponding fixation wires can be inserted to pin contact surface 144 to proximal bone portion 250. Offsetting the one or more fixation openings from the longitudinal axis of the shaft and/or center of contact surface 144 may be useful to avoid positioning fixation wires in the pathway that can interfere with a drill and/or screw guided by screw targeting guide 110.

In some examples, body 146 defining one or more contact surfaces 144 includes at least one proximal fixation aperture 150A and at least one distal fixation aperture 152A. In use when contact surface 144 is in contact with proximal bone portion 250 (e.g., the skin covering the proximal bone portion) proximal fixation aperture 150A may be located more proximally along the length of proximal bone portion 250 than distal fixation aperture 152A. When configured with multiple contact surfaces, such as first contact surface 144A in second contact surface 144B, body 146 may include fixation apertures extending through contact surfaces. For example, first contact surface 144A may include a proximal fixation aperture 150A and a distal fixation aperture 152A each configured to receive a fixation wire for pinning the first contact surface to the proximal bone portion. Second contact surface 144B may include a proximal fixation aperture 150B and a distal fixation aperture 152B each configured to receive a fixation wire for pinning the second contact surface to the proximal bone portion.

In use, the clinician may select any combination of one or more fixation apertures through which to insert corresponding fixation wires for pinning body 146 and the one or more contact surfaces 144 defined thereby to proximal bone portion 250. The clinician may insert a fixation wire through at least one fixation aperture extending through first contact surface 144A and through at least one fixation aperture extending through second contact surface 144B. The clinician may select whether to use a comparatively proximal or comparatively distal fixation aperture based on the expected positioning of a fixation wire extending there through relative to the underlying anatomy. For example, in instances where body 146 may extend across the tarsometatarsal joint and/or partially across the medial cuneiform, the clinician may use a comparatively distal fixation aperture to pin body 146 two proximal bone portion 250 to avoid inserting a pin into the tarsometatarsal joint and/or medial cuneiform if using a comparatively proximal fixation aperture.

The fixation apertures defined by body 146 and/or extending through one or more contact surfaces 144 can define a variety of different pin insertion trajectories relative to proximal bone portion 250. In some examples, a fixation aperture extending through first contact surface 144A defines a pin insertion trajectory that is angled (nonparallel) relative to a pin insertion trajectory defined by a fixation aperture extending through second contact surface 144B. Additionally or alternatively, one or more fixation apertures extending through first contact surface 144A and/or second contact surface 144B may define pin insertion trajectories that angle a pin inserted therethrough generally toward a center of proximal bone portion 250 in a dorsal-to-plantar direction. This can help prevent a pin inserted through the fixation aperture from missing engagement with proximal bone portion 250 either plantarly or dorsally. FIG. 8C is an illustration of an example configuration of body 146 defining first and second contact surfaces 144A, 144B showing example fixation aperture pin insertion trajectories with pins inserted through both contact surfaces.

As noted above with respect to FIG. 4, the example technique may include engaging a bone positioning and screw targeting instrument with proximal bone portion 250 and/or distal bone portion 252, including inserting an intramedullary insertion body associated with the instrument into proximal bone portion 250 (step 14 in FIG. 4). In some examples, the clinician forms a pocket through the cut end face of proximal bone portion 250 configured to receive intramedullary insertion body 106 before inserting the intramedullary insertion body in the pocket. The pocket can create an opening sized and oriented to receive and guide intramedullary insertion body 106 into the medullary cannel of proximal bone portion 250. If performing such a procedural step, the clinician can use a broach or other instrument to form the pocket. In other applications, the clinician inserts intramedullary insertion body 106 directly into the distal cut end face of proximal bone portion 250 (by manipulating instrument 100) without first forming a receiving pocket.

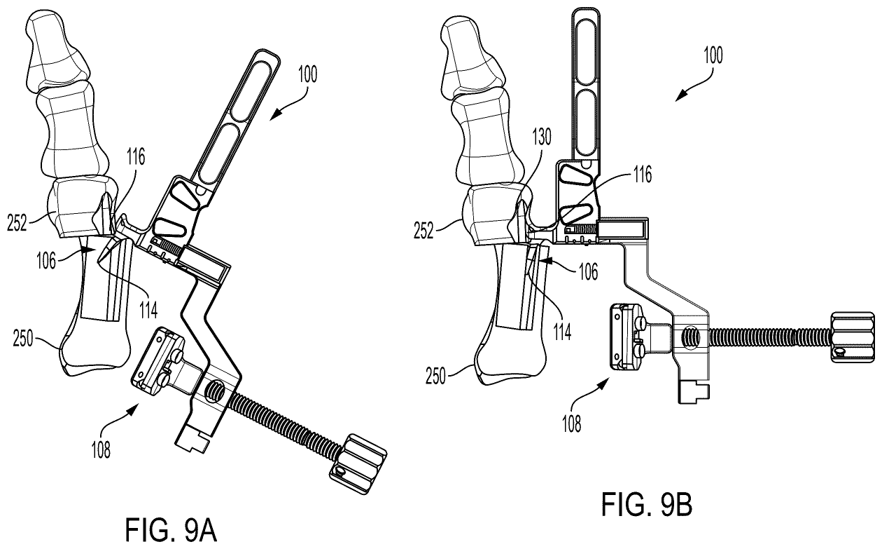

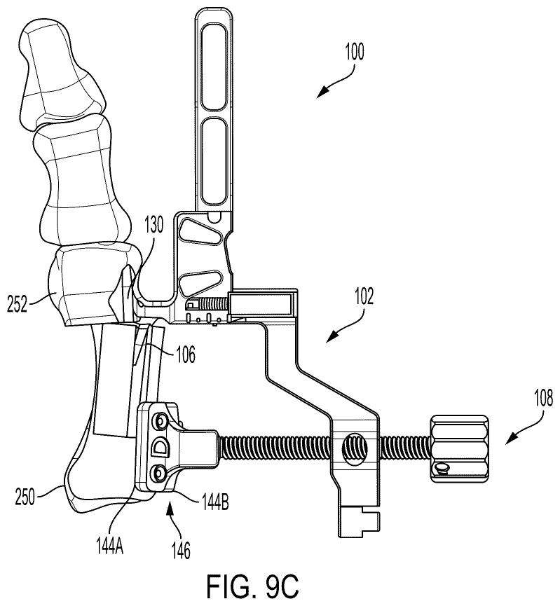

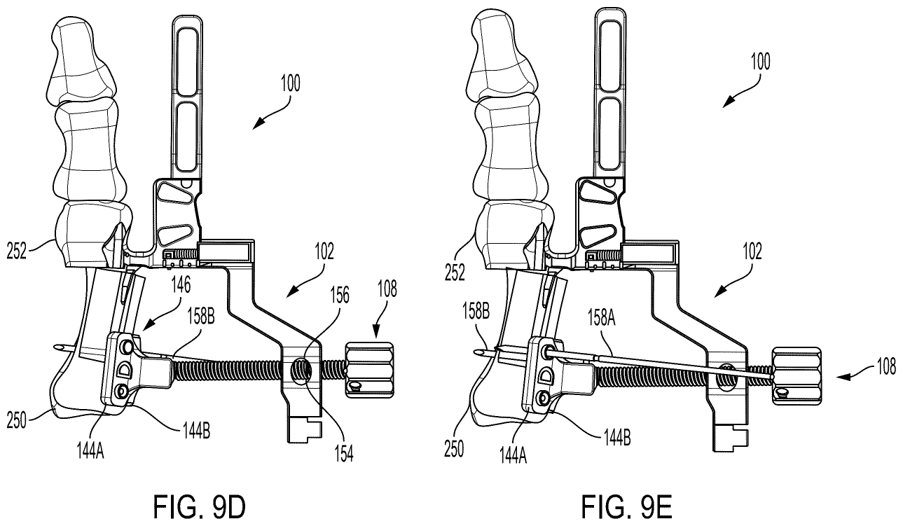

FIGS. 9A and 9B are dorsal-to-plantar views illustrating example procedure steps for inserting intramedullary insertion body 106 into proximal bone portion 250. With reference to FIGS. 9A and 9B, after cutting metatarsal into a proximal bone portion 250 and distal bone portion 252, the clinician can insert intramedullary insertion body 106 attached to instrument 100 through the cut end face of proximal bone portion 250. Intramedullary insertion body 106 may initially be inserted at an angle (FIG. 9A) from a medial side of proximal bone portion 250 (e.g., with second end 116 of intramedullary insertion body 106 positioned more immediately than first end 114 of the intramedullary insertion body). Insertion of intramedullary insertion body 106 into the cut end face of proximal bone portion 250 can cause distal bone portion 252 to be pushed laterally away from proximal bone portion 250 in the transverse plane. Instrument 100 can then be rotated to rotate intramedullary insertion body 106 to be parallel to the longitudinal axis of proximal bone portion 250, e.g., moving second end 116 of the intramedullary insertion body laterally and first and 114 of the intramedullary insertion body medially (FIG. 9B).

As intramedullary insertion body 106 is positioned through the cut end face of proximal bone portion 250 and/or instrument 100 is rotated to bring the longitudinal axis of intramedullary insertion body 106 substantially parallel to the longitudinal axis of proximal bone portion 250, distal contact surface 130 of instrument 100 can be positioned in contact with distal bone portion 252. For example, distal contact surface 130 can be positioned in contact with a medial (e.g., dorsal-medial) side of distal bone portion 252 (e.g., the skin covering the distal bone portion), as shown for example in FIG. 9B.

In some examples, intramedullary insertion body 106 is sized to be press-fit into the intramedullary canal of proximal bone portion 250, e.g., such that the clinician cannot substantially or at all translate or shift the intramedullary insertion body in the intramedullary canal once inserted. In these examples, the width of intramedullary insertion body 106 may be sized relative to the intramedullary canal to have a size effective to position the intramedullary insertion body extending across the entire intramedullary canal (e.g., with the body in contact with a cortical wall of the bone portion on one widthwise side of the body and also in contact with a cortical wall of the bone portion on an opposite widthwise side of the body).

In other examples, intramedullary insertion body 106 may be sized smaller than the intramedullary canal into which the body is inserted. For example, the width of intramedullary insertion body 106 may be sized smaller than the diameter of the intramedullary canal of proximal bone portion 250 (e.g., such that there is an offset from the cortical wall of the bone portion into which the body is inserted on one or both widthwise sides of the body). As a result of the configuration, in some examples, the clinician may translate or shift intramedullary insertion body 106 in the intramedullary canal once inserted. The clinician can shift intramedullary insertion body 106 in the intramedullary canal by shifting distal metatarsal bone 252 portion relative to proximal bone portion 250 while the intramedullary insertion body remains in a substantially stationary location and/or by shifting the intramedullary insertion body relative to the intramedullary canal while the bone portion remains substantially stationary. In either case, under sizing intramedullary insertion body 106 relative to the intramedullary canal can allow the clinician to rotate, translate, and/or otherwise move the intramedullary insertion body relative to the intramedullary canal once inserted.

The example technique of FIG. 4 also involves engaging a transverse plane bone positioning device 108 with proximal bone portion 250 (step 18 in FIG. 4). FIGS. 9C-9E are dorsal-to-plantar views illustrating example procedure steps for engaging a transverse plane bone positioning device with proximal bone portion 250. With reference to FIG. 9C, the clinician can engage the knob or other actuator or drive mechanism operatively connected to contact surface 144 to advance the contact surface relative to body 102 of instrument 100 toward proximal bone portion 250. When instrument 100 is positioned on a medial side of the metatarsal on which the procedure is being performed, the clinician can engage transverse plane bone positioning device 108 to move contact surface 144 laterally until the contact surface presses against a medial side of proximal bone portion 250 (e.g., the skin covering the medial side of the proximal bone portion). The clinician may perform an initial tightening in which contact surface 144 presses against proximal bone portion 250 with a first amount of force, e.g., to initially stabilize instrument 100. The clinician may then further advance contact surface 144 laterally, e.g., causing the contact surface to press against proximal bone portion 250 with a second amount of force greater than the first amount of force.

With reference to FIG. 9D, in some configurations, bone positioning device 108 includes an indicator 154 to help indicate to the clinician a minimum and/or maximum distance contact surface 144 should be advanced relative to body 102 of the instrument. For example, indicator 154 may be a line or other marking along a translatable feature of bone positioning device 108 (e.g., on the shaft of the bone positioning device) that moves relative to body 102 as contact surface 144 is advanced laterally relative to the body. The clinician may be instructed to advance contact surface 144 at least until indicator is aligned with or passes a corresponding reference indicator 156 defined by body 102. Additionally or alternatively, the clinician may be instructed to advance contact surface 144 a distance less than a threshold distance indicated by the same or different reference indicator 156 defined by body 102. In FIG. 9D, for example, reference indicator 156 is an opening extending through a wall surface of body 102. The clinician may advance contact surface relative to body 102 as long as indicator 154 is positioned within the opening providing reference indicator 156.

In either case, the clinician can engage proximal bone portion 250 with bone positioning device 108. In some configurations, the clinician advances body 146 on the end of a shaft of the bone positioning device laterally until first contact surface 144A contacts proximal bone portion 250 (e.g., the skin covering the proximal bone portion) and second contact surface 144B also contacts proximal bone portion 250 (e.g., the skin covering the proximal bone portion). First contact surface 144A may be positioned contacting a dorsal-medial and/or dorsal surface of proximal bone portion 250. Second contact surface 144B may be positioned contacting a plantar-medial and/or plantar surface of proximal bone portion 250. After initial contact, the clinician can further advance body 146 laterally to apply a lateral-directed force to proximal bone portion 250. Depending on the arrangement of instrument 100 relative to proximal bone portion 250, the one or more contact surfaces 144 defined by the instrument may be positioned in contact with a proximal half of the proximal bone portion, such as a proximal third, or proximal quarter of the bone portion along the length of the bone portion.

In some applications, the clinician may pin the bone positioning device 108 to proximal bone portion 250. For example, the clinician may insert a pin through fixation aperture operatively connected to contact surface 144 to pin the contact surface to the underlying bone. With reference to FIGS. 9D and 9E, the clinician may insert one or more pins 158A through a proximal and/or distal fixation aperture extending through first contact surface 144A in before or after insert one or more pins 158B through a proximal and/or distal fixation aperture extending through second contact surface 144B. In the illustrated arrangement, first pin 158A it is inserted through a distal fixation aperture extending through first contact surface 144A and second pin 158B it is inserted through a distal fixation aperture extending through second contact surface 158B. Other arrangements or combinations of fixation apertures and pin placements can be used.

In any arrangement, the one or more pins may be inserted along a pin insertion trajectory that advances the pin through at least one cortical wall surface of proximal bone portion 250. In some examples, at least one pin (e.g., optionally all pins inserted) are inserted bicortically such that the pin extends through to cortical wall surfaces of proximal bone portion 250. For example, a pin may be inserted through a medial, dorsal-medial and/or dorsal cortical wall a proximal bone portion 250 and project out a lateral, dorsal-lateral, plantar-lateral, and/or plantar cortical wall of the proximal bone portion. Additionally or alternatively, a pin may be inserted through a medial, plantar-medial and/or plantar cortical wall a proximal bone portion 250 and project out a lateral, dorsal-lateral, and/or dorsal cortical wall of the proximal bone portion.

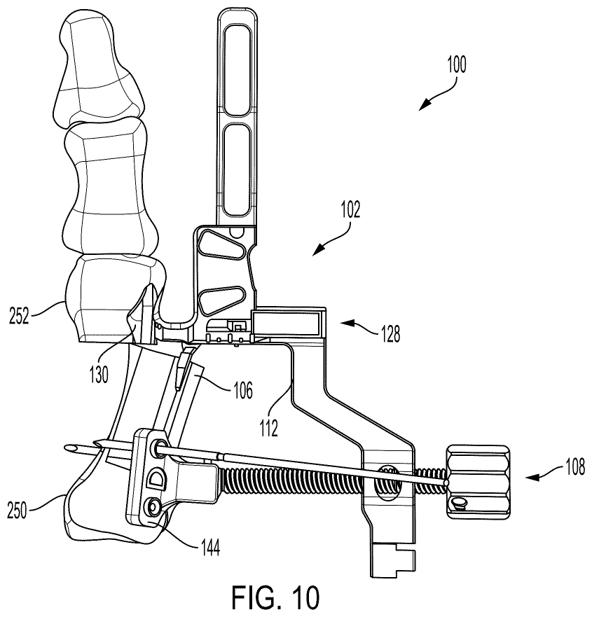

The example technique of FIG. 4 also involves adjusting a transverse plane position of distal bone portion 252 relative to proximal bone portion 250 (step 20 in FIG. 4). FIG. 10 is a dorsal-to-plantar view illustrating an example procedure step for moving distal bone portion 252 relative to proximal bone portion 250. In the illustrated example, the proximal bone portion 250 is moved medially relative to distal bone portion 252 in the transverse plane while the distal bone portion is held at a substantially stationary position to create relative offset between the two bone portions. In other applications, distal bone portion 252 may be moved relative to a stationary or moving proximal bone portion 250 without departing from the scope of disclosure.

With reference to FIG. 10, the clinician can engage intramedullary insertion body translation mechanism 128 to move intramedullary insertion body 106 inserted into the cut end face of proximal bone portion 250 medially. This can provide a pulling force causing the distal end of proximal bone portion 250 to move medially (angle medially outwardly) about a rotational axes or pivot point defined by the proximal end of proximal bone portion 250 (the tarsometatarsal joint). Bone positioning device 108 and, more particularly, contact surface 144 can provide a laterally directed counterforce adjacent the proximal end of proximal bone portion 250. As intramedullary insertion body 106 is advanced medially toward body 102 of instrument 100, distal contact surface 130 of the body can press against the medial side of distal bone portion 252. This can also provide a laterally directed counterforce adjacent the proximal end of distal bone portion 252, e.g., helping the distal bone portion remain in a substantially stationary transverse plane location while the distal end of proximal bone portion 250 is pulled medially.

Accordingly, before, after, and/or while actuating bone positioning device 108, the clinician can engage intramedullary insertion body translation mechanism 128. For example, the clinician can engage a driver or knob to turn a threaded shaft attached to intramedullary insertion body 106 to draw the insertion body toward support surface 112 of instrument 100. This can cause intramedullary insertion body 106 to move medially within the medial canal of proximal bone portion 250. As a result, distal bone portion 252 can be shifted further laterally relative to the proximal bone portion within the transverse plane.

The cut end faces of distal bone portion 252 and proximal bone portion 250 may be moved away from each other in the transverse plane a distance effective so the medial-most half or less of distal bone portion 252 is positioned over and/or in contact with a lateral portion of the cut end face of proximal bone portion 250, such as the medial-most third or less of the distal bone portion, the medial-most quarter or less of the distal bone portion, or the medial-most fifth or less of the distal bone portion. The cut end faces of distal bone portion 252 and proximal bone portion 250 may be moved away from each other in the transverse plane a distance effective to substantially maximize the offset between the cut end faces in the transverse plane while still maintain at least some contact between the cut end faces. This can help maximize the amount of correction provided by the procedure.

While technique of FIG. 4 has been described in conjunction with FIG. 10 as you utilizing intramedullary insertion body translation mechanism 128 and/or bone positioning device 108 to facilitate transverse plane movement of proximal bone portion 250 and distal bone portion 252 relative to each other, in some applications the technique, the clinician may additionally or alternatively perform transverse plane correction by hand. For example, the clinician may additionally or alternatively grasp a pin inserted into distal bone portion 252 and/or a pin inserted into proximal bone portion 250 and manipulate the position of the pins and/or corresponding bone portions in the transverse plane.

The example technique of FIG. 4 also involves moving distal bone portion 252 relative to proximal bone portion 250, e.g., to correct a deformity such as a bunion deformity (step 20 in FIG. 4). For example, the clinician can shift distal bone portion 252 in the transverse plane (e.g., to move the distal bone portion laterally), rotate the distal bone portion in the frontal plane, and/or shift the distal bone portion in the sagittal plane. While the example of FIG. 4 describes an example procedure order in which distal bone portion 252 is realigned following insertion of the intramedullary stem into proximal bone portion 250, it should be appreciated the procedure is not limited to this order.

For example, realignment of distal bone portion 252 may occur through various stages of the surgical procedure. The distal bone portion 252 may be moved in the transverse plane (e.g., laterally) relative to proximal bone portion 250 prior to and/or while moving the distal bone portion 252 in the frontal plane and/or sagittal plane. For example, after cutting first metatarsal 210 into the proximal bone portion 250 and distal bone portion 252, the clinician can shift distal bone portion 252 laterally to at least partially expose the cut end face of proximal bone portion 250. The clinician can grasp a K-wire inserted into distal bone portion 252 (e.g., a K-wire used to secure the bone preparation guide to the distal bone portion, with the K-wire remaining in the bone portion after removing the bone preparation guide) to move the distal bone portion laterally.

The distance distal bone portion 252 is moved laterally in the transverse plane relative to proximal bone portion 250 may be controlled by the clinician. In some examples, distal bone portion 252 is shifted laterally in the transverse plane so less than half of the cut end face of the distal bone portion is positioned over and/or in contact with the cut end face of the proximal bone portion. For example, distal bone portion 252 may be shifted laterally so the medial-most half or less of the distal bone portion is positioned over and/or in contact with a lateral portion of the cut end face of proximal bone portion 250, such as the medial-most third or less of the distal bone portion, the medial-most quarter or less of the distal bone portion, or the medial-most fifth or less of the distal bone portion.

Distal bone portion 252 can be moved in one or more other planes relative to proximal bone portion 250 in addition to or in lieu of moving the bone portion in the transverse plane. For example, distal bone portion 252 may be rotated in the frontal plane and/or plantar flexed or dorsiflexed in the sagittal plane.

The clinician may move distal bone portion 252 in one or more planes with and/or without the aid of a bone positioning device. In some examples, the clinician grasps one or more K-wires inserted into distal bone portion 252 (e.g., extending percutaneously out of the skin of the patient) and manipulates the position of the distal bone portion in one or planes. Additionally or alternatively, the clinician may engage a bone positioning device with distal bone portion 252 that can be controlled (e.g., actuated) to move the distal bone portion in one or planes.

In some applications, the clinician may shift rotate distal bone portion 252 in the frontal plane and/or shift the distal bone portion in the sagittal plane relative to proximal bone portion 250 in addition to or in lieu of adjusting a relative position of the bone portions in the transverse plane. To move distal bone portion 252 in the frontal plane, the clinician may grasp the bone portion (optionally via a K-wire inserted into the bone portion) and rotate the bone portion in the frontal plane. Additionally or alternatively, the clinician may grasp the bone portion (optionally via a K-wire inserted into the bone portion) and dorsiflex or plantar flex the bone portion to move the bone portion in the sagittal plane.

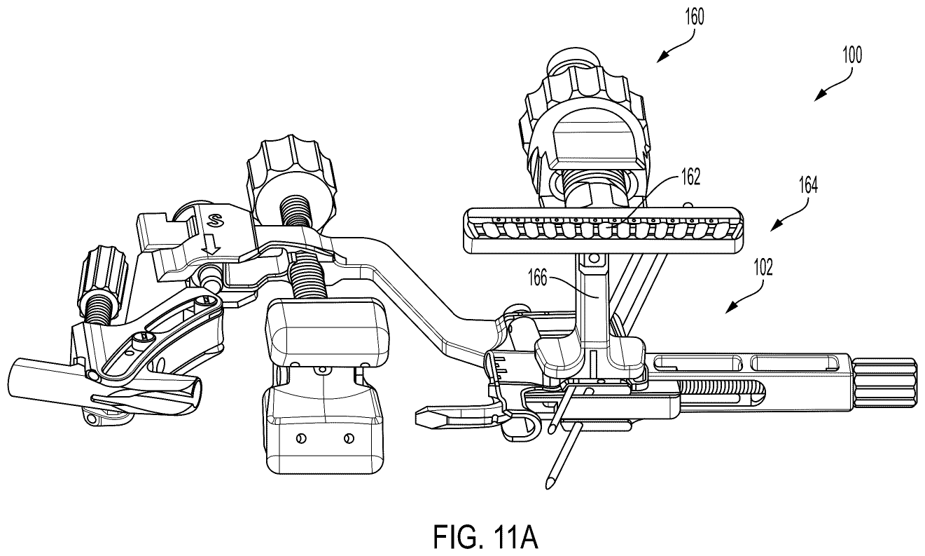

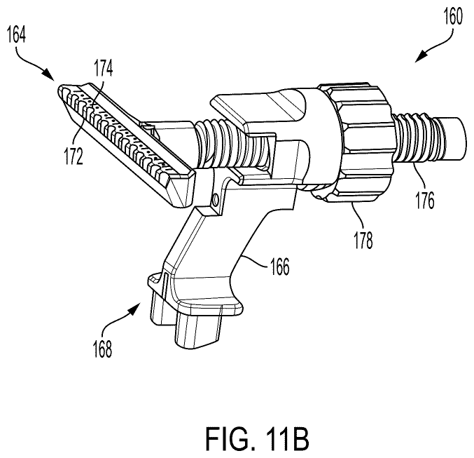

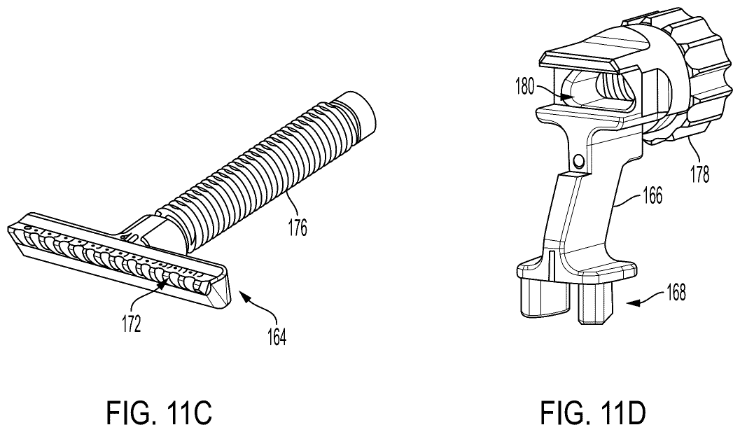

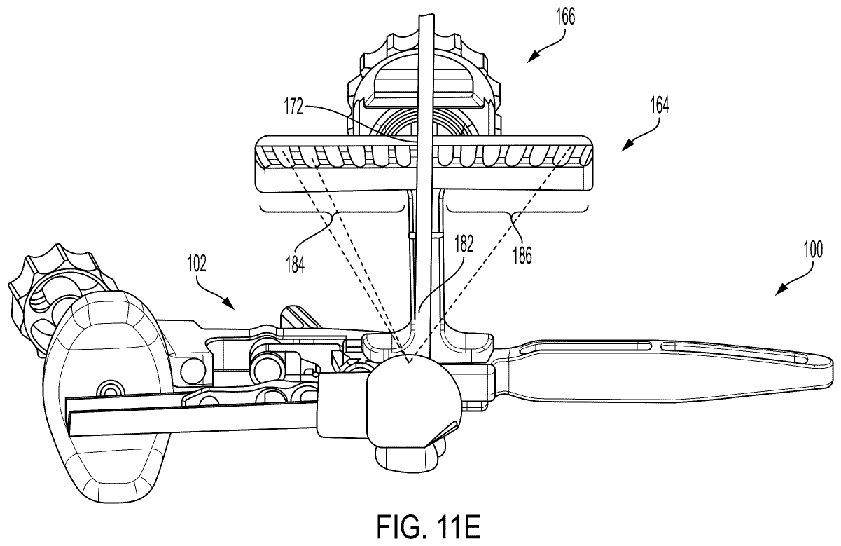

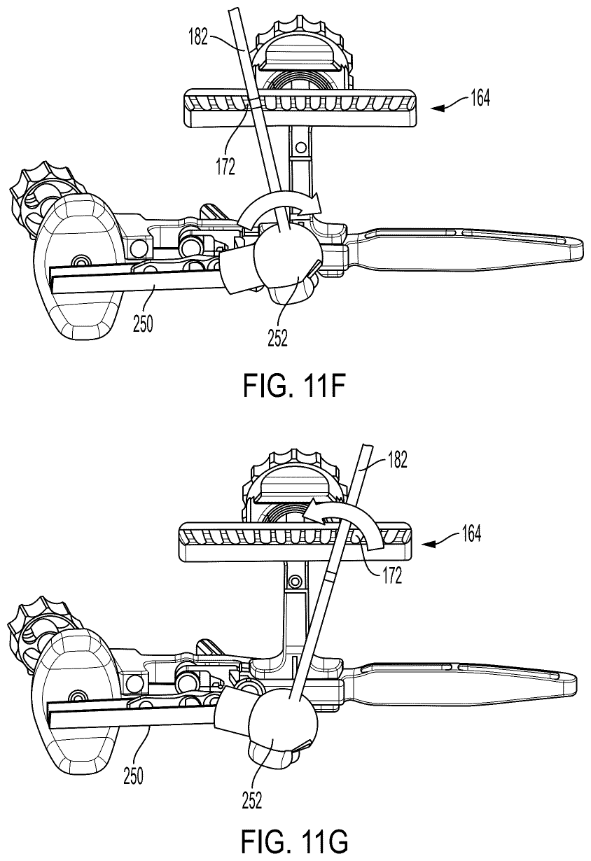

In some examples, instrument 100 is configured with a bone positioning device operable to apply a force to distal bone portion 252 to controllably reposition the bone portion in the frontal plane (in addition to or in lieu of configuring the bone positioning device to apply a force to controllably position distal bone portion 252 relative to proximal bone portion 250 in the transverse plane). FIGS. 11A-11G (collectively “FIG. 11”) are different views of an example bone positioning device 160 that can be used with instrument 100 to control repositioning of a bone portion (e.g., distal bone portion 252) in the frontal plane. Bone positioning device 160 can be referred to as a frontal plane bone positioning device and/or a second bone positioning device, when instrument is configured with a transverse plane bone positioning device and/or first bone positioning device 108. FIG. 11A is a lateral-side view of instrument 100 showing an example configuration of bone positioning device 160 operatively engaged with body 102 of instrument 100.

As shown in the example of FIG. 11, bone positioning device 160 can include at least one wire receiving opening 162. A wire (e.g., K-wire) inserted into distal bone portion 252 can be received in wire receiving opening 162 to operatively connect the bone to bone positioning device 160. The body 164 defining wire receiving opening 162 can be laterally offset from the main body 102 of instrument 100. For example, bone positioning device 360 may include a dorsally extending support arm 166 and/or a laterally extending support arm that positions the body defining wire receiving opening 162 dorsally and/or laterally offset from the body 102 (e.g., when instrument 100 is engaged on the medial side of the bone portions). In some applications, body 164 and/or contact surface 144 is formed of a radiolucent material, such as plastic, to facilitate visualization through the body under fluoroscopic or other radiographic imaging.

FIG. 11B is a perspective view of an example configuration of bone positioning device 160 operable to apply a force to distal bone portion 252 to controllably reposition the bone portion in the frontal plane. Bone positioning device 160 can engage a wire (e.g., K-wire) inserted into distal bone portion 252 to operatively engage the bone positioning device with the bone. Bone positioning device 160 can be permanently or detachably connected to body 102 of instrument 100. For example, bone positioning device 160 and body 102 may have complementary male and female connection features that allow the two components to be joined together. In the illustrated example, bone positioning device 160 is illustrated as including a male connector 168 that is insertable into a complementary female receiving opening 170 of body 102 (FIG. 7) and can be frictionally retained therein. Other connection configurations can be used without departing from the scope of the disclosure.

Bone positioning device 160 in the example of FIG. 11B can include a wire receiving body 164 configured to receive a wire inserted into distal bone portion 252. Wire receiving body 164 can define one or more grooves 172 (each of which defines a wire receiving opening 162) into which the wire can be received (e.g., with the side wall defining the groove at least partially extending about the wire received in the groove). For example, wire receiving body 164 may include a plurality of grooves 172 arrayed side-by-side with respect to each other (e.g., with different grooves being spaced from each other in a proximal to distal direction, when instrument 100 is in use). Each of the plurality of grooves may define a different location that can receive the wire inserted into distal bone portion 252. Configuring wire receiving body 164 with multiple different grooves or wire receiving locations offset from each other can be useful to provide flexibility depending on where the wire inserted into distal bone portion 252 is positioned relative to the wire receiving body.

In the illustrated example of FIG. 11B, wire receiving body 164 is shown in the form of a rake having multiple adjacent wire receiving grooves 172. For example, wire receiving body 164 may define a plurality of teeth 174 arrayed in a row. The plurality of grooves 172 may be defined by the plurality of teeth 174, with each groove being a recess relative to the projecting surfaces defined by adjacent teeth 174. Each of the grooves may be sized and shaped relative to the wire inserted into distal bone portion 252. For example, each of the grooves 172 may define a circular shape (e.g., semi-circular wall portion) in may be sized as large or larger than the diameter of the wire inserted into distal bone portion 252. Grooves having other sizes and shapes may also be used in the effective to receive the wire inserted into distal bone portion 252. The specific number of wire receiving grooves 172 defined by wire receiving body 164 can vary, e.g., from 1 to 50, such as from 2 to 40, or 5 to 25.

Wire receiving body 164 can be offset from the body 102 of instrument 100. For example, bone positioning device 164 may include an arm 166 that is configured to engage with and/or extend from instrument 100. Wire receiving body 164 can be connected to and/or carried by arm 166. For example, wire receiving body 164 can be operably connected to a shaft 176 which, in turn, is operably connected to arm 166. Wire receiving body 164 can translate relative to arm 166 and/or body 102 of instrument 100 via shaft 176. In some configurations, wire receiving body 164 is movable along shaft 176. In other configurations, where receiving body 164 is movable with shaft 176. For example, in the illustrated configuration, wire receiving body 164 is positioned on an end of shaft 176 and moves as the shaft is translated relative to arm 166. Shaft 176 may be defined by a threaded rod, rack and pinion, ratchet, and/or other mechanical linkage that allows the position of wire receiving body 164 to be moved. In some examples, bone positioning device 160 includes an actuator 178 (e.g., rotatable knob, screw drive) that a clinician can engage to control the position of wire receiving body 164 relative to instrument 100 and/or arm 166 of the bone positioning device.

FIGS. 11C and 11D are perspective views of the example components of bone positioning device 160 shown disassembled from each other. In particular, FIG. 11C illustrates an example configuration of wire receiving body 164 and shaft 176, while FIG. 11D illustrates an example configuration of arm 166 and actuator 178. In the example of FIGS. 11C and 11D, shaft 176 is illustrated as defining a threaded portion that is configured to be inserted into a corresponding receiving opening 180 of the base of arm portion 166 of the bone positioning device. In some examples, shaft 176 and receiving opening 180 define an asymmetric cross-sectional shape (e.g., a generally D-shaped body and opening) to prevent misalignment of the shaft and wire receiving body carrying thereby relative to arm 166. Receiving opening 180 can define a channel for receiving shaft 176 that can constrain movement of the shaft in a linear direction (e.g., medially to laterally) while reducing or eliminating upward or downward pitching (e.g., dorsal or plantar) and/or side to side pivoting (e.g., distal or proximal).

Each groove 172 of receiving body 164 may define a longitudinal length extending parallel to the length of the wire inserted into distal bone portion 252 (when the wire is received in the groove). For example, each groove 172 of wire receiving body 164 may define a longitudinal length extending in a dorsal to plantar direction, when bone positioning device 160 is engaged with instrument 100 and in use. The longitudinal length of each groove 172 of wire receiving body 164 may be parallel to each other of the plurality of grooves of the wire receiving body. Alternatively, the longitudinal length of one or more grooves 172 of wire receiving body 164 may be angled relative to the longitudinal length of one or more other of the plurality of grooves 172 of the wire receiving body. Angling the longitudinal length of grooves 172 relative to each other may be useful to provide flexibility for controlling and adjusting the sagittal plane positioning of distal bone portion 252.

FIG. 11E illustrates an example configuration of bone positioning device 160 attached to and extending from instrument 100 with the instrument engaged with both proximal bone portion 250 and distal bone portion 252. As illustrated, a wire 182 is inserted into distal bone portion 252 and received by one of the plurality of wire-receiving openings / grooves 172 of wire receiving body 164. Bone positioning device 160 in this example is configured to control an angle of distal bone portion 252 in the sagittal plane relative to proximal bone portion 250 by receiving wire 182 in different ones of the plurality of grooves 172. For example, the longitudinal length of plurality of grooves 172 may extend at different angles relative to each other. In some configurations, one of the plurality of grooves 172 (e.g., a groove at or around the middle of the array of different grooves) may extend orthogonally (at 90°) relative to main body 102 of instrument 100 and/or parallel to the dorsal-to-planar direction of the metatarsal bone portions. Grooves 172 offset from the parallel groove may be angled in the dorsal-to-planar direction, with the extent of angulation increasing moving away from the middle of the array of grooves. Wire receiving body 164 may include a first plurality of grooves 184 angled in a first sagittal plane direction and a second plurality of grooves 186 angled in a second sagittal plane direction opposite the first sagittal plane direction. The two sets of grooves may be positioned on opposite sides of the center of the array of grooves. In some configurations, the longitudinal lengths of the plurality of grooves 172 are angled to define wire receiving axes that converge at an apex point. In other configurations, the longitudinal lengths of the plurality of grooves 172 are angled to define wire receiving axes that do not converge.

FIGS. 11F and 11G illustrate bone positioning device 160 from FIG. 11E showing wire 182 positioned in different example grooves of wire receiving body 164 to adjust the sagittal plane angle of distal bone portion 252. FIG. 11F illustrates wire 182 received in a groove 172 that plantar biases distal bone portion 252. Inserting wire 182 into a plantar biased groove can cause the proximal end of distal bone portion 252 to angle plantarly (downwardly) and/or the distal end of the distal bone portion to angle dorsally (upwardly). The clinician can move the position of wire 182 to a different one of the plurality of grooves to adjust the sagittal plane angulation of distal bone portion 252 (e.g., optionally moving the wire to a position that causes the distal bone portion to be substantially coplanar with the proximal bone portion 250). FIG. 11G illustrates wire 182 received in a groove 172 that dorsal biases distal bone portion 252. Inserting wire 182 into a dorsal biasing groove can cause the proximal end of distal bone portion 252 to angle dorsally (upwardly) and/or the distal end of the distal bone portion to angle plantarly (downwardly). Again, the clinician can move the position of wire 182 to a different one of the plurality of grooves to adjust the sagittal plane angulation of distal bone portion 252.

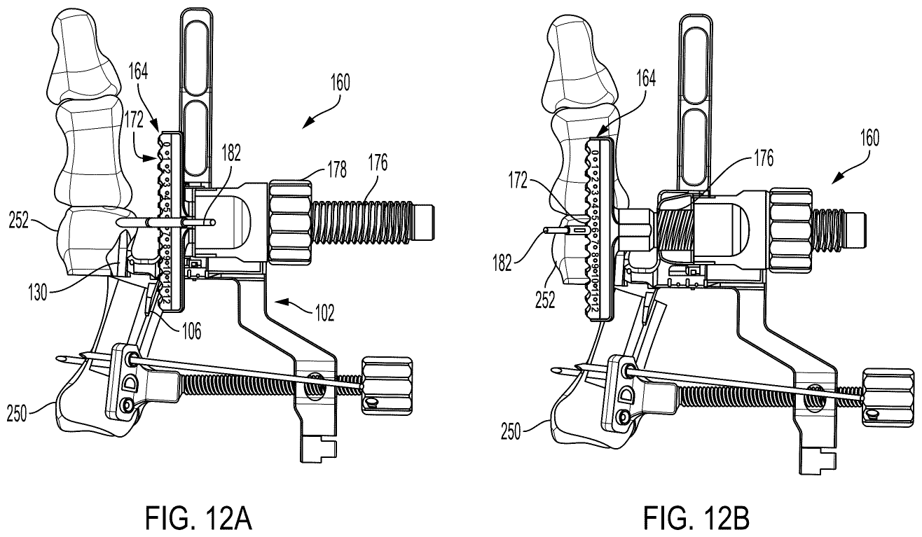

The example technique of FIG. 4 can involve adjusting frontal plane rotational position of distal bone portion 252 and/or the sagittal plane position of the distal bone portion relative to proximal bone portion 250, e.g., using bone positioning device 160 of instrument 100 (step 22 in FIG. 4). Before, after, and/or while moving distal bone portion 252 relative to proximal bone portion 250 (e.g., by moving the proximal bone portion medially), distal bone portion 252 may be moved in the frontal plane and/or sagittal plane using a frontal plane and/or sagittal plane bone positioning device, such as bone positioning device. FIGS. 12A and 12B (collectively “FIG. 12”) are dorsal-to-plantar views illustrating example procedure steps for engaging a frontal plane and/or sagittal plane bone positioning device 160 with distal bone portion 252 and using the device to adjust a position of the bone portion.

As shown in FIG. 12, bone positioning device 160 can be connected to the body 102 of instrument 100 (in configurations in which the bone positioning device 160 is attachable to and detachable from body 102). When configured to be detachable from body 102, bone positioning device 160 may be connected to body 102 of instrument 100 throughout the procedure or may be attached during the procedure when ready to utilize bone positioning device 160. Accordingly, while example procedural steps have been illustrated and described above without bone positioning device 160 being attached to instrument 100, it should be appreciated that the bone positioning device may be connected to the instrument when performing other procedure steps as described herein.

In either case, in use, wire receiving body 164 of bone positioning device 160 can be advanced until a wire 182 inserted (e.g., percutaneously) into distal bone portion 252 is received in one of the plurality of grooves 172 of the wire receiving body. For example, the clinician can engage actuator 178 to translate shaft 176 relative to support arm 166, causing wire receiving body 164 to advance until the wire receiving body contexts wire 182. Because wire 182 may receive and transmit a comparatively high force to control positioning of distal bone portion 252, the wire selected to be used as wire 182 may have a sufficiently large diameter for the application. In various examples, wire 182 may have a diameter of at least 1.0 mm, such as at least 1.2 mm, at least 1.6 mm, at least 2.0 mm, at least 2.2 mm, or at least 2.4 mm.

In the illustrated arrangement of FIG. 11, body 102 of instrument 100 is shown positioned on a medial side of the proximal bone portion 250 and on a medial side of distal bone portion 252 with intramedullary insertion body 106 inserted into proximal bone portion 250 and distal contact surface 130 flush against distal bone portion 252. When so positioned, arm 166 of bone positioning device 160 can be positioned to extend in a dorsal direction from body 102 and shaft 176 of the bone positioning device can be positioned to extend in a lateral direction from the arm. As a result, bone positioning device 160 can apply a laterally-directed force to wire 182 to move the distal bone portion 252 in at least a frontal plane.

The clinician can advance wire receiving body 164 by controlling actuator 178 until distal bone portion 252 is at a desired frontal plane rotation position. In some instances, this involves repositioning the sesamoid bones plantarly and parallel to ground under distal bone portion 252. In some examples, the clinician may over rotate distal bone portion 252 such that the sesamoid bones under the distal bone portion are shifted from directly plantar to being angled at least partially medially. Over rotation may be beneficial if spring back or derotation is anticipated later in the procedure. In some examples, the clinician rotates distal bone portion 252 in the frontal plane to substantially realign the rotational position of the distal bone portion and/or sesamoid bones back to their normal anatomical frontal plane rotation position, e.g., such as the position as observed in normal patient population not experiencing a bunion deformity.

Before, during, and/or after moving distal bone portion 252 in the frontal plane, the clinician can additionally or alternative move the distal bone portion 252 in the sagittal plane to correct the sagittal plane alignment of the metatarsal bone portion. For example, the clinician can move wire 182 from one of the plurality of grooves 172 to a different one of the plurality of grooves 172. Where the grooves are angled relative to each other, shifting wire 182 proximally or distally along the array of grooves from one group to a different groove can cause the angular orientation of wire 182 to change, and correspondingly change in the sagittal plane angulation of distal bone portion 252. This can change the angular orientation of the cut end face of distal bone portion 252 relative to the cut end face of proximal bone portion 250 in the sagittal plane. The clinician may adjust the angular orientation of distal bone portion 252 so the distal bone portion is substantially in line with (e.g., coaxial with) proximal bone portion 250 and is not pitched dorsally or plantarly. For example, during the procedure, tissue surrounding proximal bone portion 250 and/or distal bone portion 252 can introduce misalignments to distal bone portion 252 as other portions of the procedure are performed (e.g., resulting in distal bone portion 252 angling proximally or distally) which can be corrected during sagittal plane adjustment.

Additionally or alternatively, the clinician can shift the entirety of distal bone portion 252 dorsally or plantarly in the sagittal plane (in addition to or in lieu of change in the angular orientation of the cut end face of the metatarsal bone portion). For example, the clinician can apply a force to wire 182 in a plantar direction, advancing the wire plantarly to move distal bone portion 252 plantarly or advance the wire dorsally to move distal bone portion 252 dorsally. The clinician can move wire 182 dorsally or plantarly while the wire is retained in one of the plurality of grooves 172. The clinician can adjust the orientation of distal bone portion 252 so the distal bone portion is substantially in line with (e.g., coaxial with) proximal bone portion 250. If desired, a securing mechanism can be used to lock wire 182 at a particular location and/or orientation relative to wire receiving body 164 to prevent inadvertent movement of the wire (and correspondingly distal bone portion 252) after the distal bone portion 252 has been moved to a desired frontal plane and/or sagittal plane position.

In clinical practice, an observed challenge during minimally invasive metatarsal correction can be unintended plantar translation of distal bone portion 252 (the metatarsal head or capital fragment), while the surgeon is performing corrective manipulation, whether by transverse-plane translation, frontal-plane derotation, sagittal-plane adjustment, or combinations thereof. As corrective forces are applied through percutaneous instruments and/or pins, the distal bone portion 252 may tend to drop or drift plantarly relative to the proximal metatarsal portion, particularly when soft-tissue tension, instrument leverage, and limited direct visualization permit subtle loss of dorsal support. This plantar displacement, if not controlled, can be a limiting factor in achieving a reproducible correction because it may compromise fragment control, alter the intended bony relationship across the osteotomy, and/or complicate subsequent steps such as accurate targeting and advancement of fixation elements. Accordingly, in some configurations, instrument 100 is configured to provide a dedicated support that applies an upward (dorsally directed) force beneath distal bone portion 252 during manipulation to help mitigate plantar drift and improve maintenance of the desired reduction while other alignment adjustments and/or fixation are performed.

A plantar support usable to resist plantar translation of distal bone portion 252 can be implemented in a variety of configurations and may be provided as a feature integrated with instrument 100 or, alternatively, as a separate accessory engageable with or used separately from instrument 100. In some examples, the plantar support is operatively connected to body 102 of instrument 100 and is movable relative to the body to position a support surface beneath distal bone portion 252 and to apply a dorsally directed support force to the distal bone portion. In other examples, the plantar support is detachably connectable to instrument 100, such as by sliding engagement, clamping engagement, or engagement with a complementary coupling feature on instrument 100, thereby allowing the plantar support to be added when desired and removed when not needed. In still other examples, the plantar support can be provided as a separate instrument that is positionable relative to instrument 100 (and/or relative to a handle, jig, or other component used during the procedure) to place a support surface beneath distal bone portion 252 without being physically attached to instrument 100, while still being useable concurrently with instrument 100 to maintain the reduction during manipulation and/or fixation.

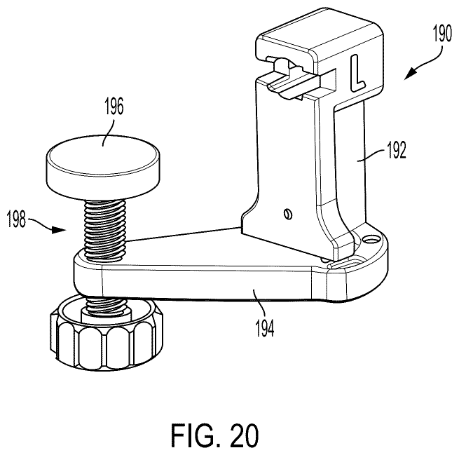

FIG. 20 is a perspective view showing an example configuration of a plantar support mechanism 190 for distal bone portion 252 that can be engaged with instrument 100. The plantar support may include a base 192 configured to interface with instrument 100, an arm 194 extending from the base to position a support surface 196 beneath distal bone portion 252, and an adjuster 198 (e.g., a threaded mechanism) operable to advance the support surface 196 dorsally or plantarly to provide controlled support.

Support surface 196 can be shaped and configured in a variety of ways to interface with tissue overlying distal bone portion 252 and to provide stable resistance to plantar translation without creating undesirable point loading. In some examples, support surface 196 is defined by a cup or platform having a generally concave profile sized to at least partially cradle an inferior aspect of distal bone portion 252, thereby increasing contact area and improving resistance to slipping during manipulation. Additionally or alternatively, support surface 196 can be substantially planar, arcuate, or saddle-shaped, and may include rounded edges and/or radiused transitions to reduce localized pressure on soft tissue. In some examples, support surface 196 includes a compliant layer or insert (e.g., elastomeric pad) to accommodate variation in anatomy and to distribute load. In further examples, support surface 196 defines one or more relief features such as a notch, slot, channel, or opening to avoid interference with pins, wires, or other instruments and/or to permit visualization. Support surface 196 may be provided in multiple sizes and/or shapes, including patient-specific shapes, and may be configured to be interchangeable with plantar support mechanism 190.

Adjuster 198 can be implemented in a variety of ways to set and/or control a position of support surface 196 relative to instrument 100 and distal bone portion 252. In some examples, adjuster 198 comprises a threaded mechanism including a screw, lead screw, or jack screw operatively coupled to support surface 196 such that rotation of an actuator (e.g., a knob or driver receptacle) advances support surface 196 dorsally toward distal bone portion 252 or retracts support surface 196 plantarly away from distal bone portion 252. In other examples, adjuster 198 comprises a rack-and-pinion mechanism, a cam mechanism, a ratchet mechanism, a wedge mechanism, or a linkage-driven slider configured to provide incremental or continuous adjustment. In some configurations, adjuster 198 provides a lock or detent to maintain a selected support position under load. In other configurations, support surface 196 is fixed in position relative to a base and arm of plantar support mechanism 190 such that the dorsal support is provided by placing the mechanism at a predetermined height and/or by selecting a particular size or configuration of the mechanism. In still other configurations, support surface 196 is movable via adjuster 198 through a defined range of travel, allowing the clinician to initially position support surface 196 beneath distal bone portion 252 and then advance the support surface to apply a controlled dorsally directed support force, optionally prior to manipulation as a preventative support or during manipulation in response to observed plantar drift.

Plantar support mechanism 190 can be permanently connected to instrument 100 or detachably connectable to instrument 100 to permit selective use during a procedure. When detachably connected, plantar support mechanism 190 may be coupled to instrument 100 in a variety of different ways, including by a sliding engagement, a dovetail or tongue-and-groove interface, a clamp, a collet, a threaded coupling, a bayonet-style connection, a latch, a pin-and-slot arrangement, a cam lock, and/or other complementary mating features configured to provide a repeatable and stable attachment. In some examples, plantar support mechanism 190 is configured to engage with a handle portion of instrument 100, thereby allowing the clinician to mount the plantar support mechanism on the handle and translate the mechanism relative to the handle until support surface 196 is positioned beneath distal bone portion 252. In some examples, such as illustrated, plantar support mechanism 190 includes a base 192 defining an opening configured to receive and slide over the handle of instrument 100, optionally with a rectangular profile to resist rotation relative to the handle and to maintain a selected orientation of arm 194 and support surface 196 during use.

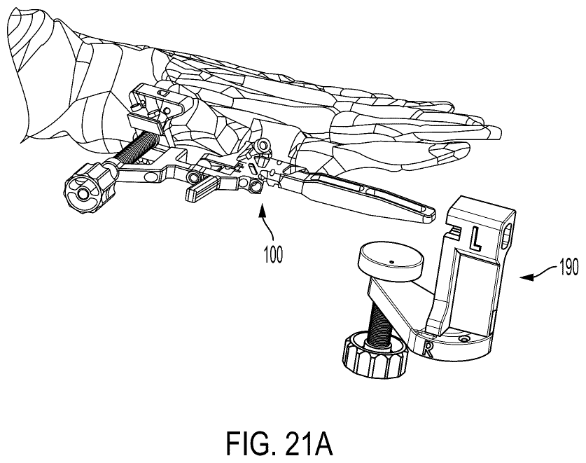

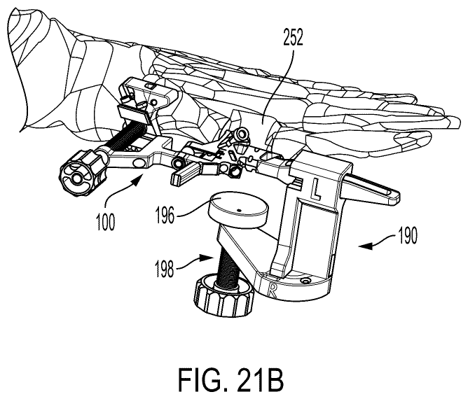

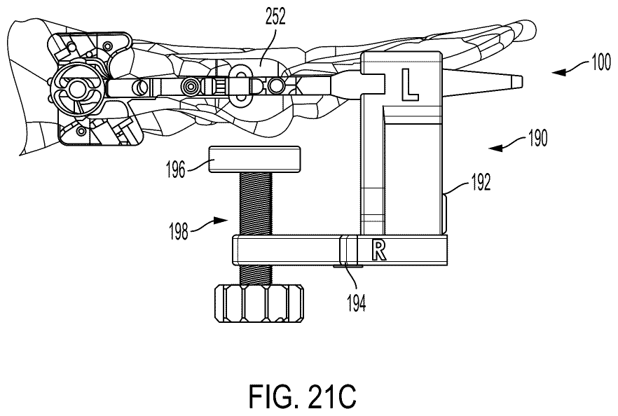

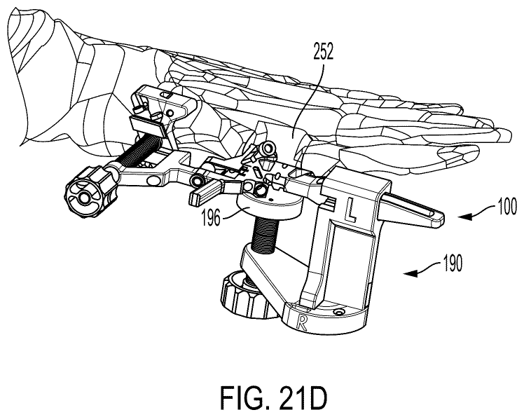

FIGS. 21A-21D illustrate example procedure steps for engaging plantar support mechanism 190 with instrument 100 and actuating plantar support mechanism 190 to engage distal bone portion 252. FIG. 21A illustrates base 192 positioned offset from and alignable with instrument 100 prior to attachment, such as with base 192 oriented to be advanced onto a handle portion of instrument 100. FIGS. 21B and 21C are perspective and medial-to-lateral side views, respectively, illustrating base 192 engaged with instrument 100 and, more particularly, slid onto and retained on the handle of instrument 100, with base 192 positioned in a proximal-to-distal direction along the handle to place support surface 196 beneath distal bone portion 252. In the illustrated arrangement, arm 194 extends from base 192 to position support surface 196 inferior to a plantar aspect of distal bone portion 252 while instrument 100 remains engaged with the metatarsal. FIG. 21D illustrates adjuster 198 actuated to advance support surface 196 dorsally into contact with the plantar side of distal bone portion 252 to provide plantar support resisting plantar translation of distal bone portion 252 during correction and/or fixation.

Plantar support mechanism 190 may be applied and used at different stages of a minimally invasive metatarsal correction procedure. In some examples, plantar support mechanism 190 is engaged with instrument 100 immediately after the instrument is inserted into the metatarsal shaft (e.g., prior to adjusting distal bone portion 252 using instrument 100). In other examples, plantar support mechanism 190 is applied after the metatarsal has been cut to form proximal bone portion 250 and distal bone portion 252, such as after distal bone portion 252 has been shifted and/or rotated relative to proximal bone portion 250 and the clinician desires to reduce or eliminate plantar drift during continued manipulation and/or fixation. In either case, plantar support mechanism 190 may remain in place throughout one or more manipulation steps and/or during screw targeting and insertion, or may be selectively added and removed as needed.

In use, plantar support mechanism 190 can be actuated to provide a stable support that resists plantar translation of distal bone portion 252 by maintaining support surface 196 in contact with, or in close proximity to, the plantar side of distal bone portion 252. In some configurations, the support provided by support surface 196 is set to a selected height to prevent unintended downward movement while allowing other adjustments (e.g., transverse-plane translation and/or frontal-plane rotation) to be performed with reduced risk of loss of reduction. In other configurations, plantar support mechanism 190 is used more actively to drive positioning of distal bone portion 252 in the sagittal plane, such as by actuating adjuster 198 to advance support surface 196 dorsally to elevate distal bone portion 252 (e.g., to reduce plantar displacement) and/or to maintain a desired sagittal-plane relationship of distal bone portion 252 relative to proximal bone portion 250 while fixation elements are placed. Accordingly, plantar support mechanism 190 may be used as a passive stabilizer, an active repositioning tool, or both, depending on the desired procedural workflow and the observed behavior of distal bone portion 252 during correction.

Additional instrument configurations and techniques can use a plantar support mechanism 190 as described herein are described in United States Patent Application No. 2025/0049443, filed Jul. 15, 2024, the entire contents of which are incorporated herein by reference.

It should be appreciated that while example techniques are described herein, including with respect to FIG. 4, the described procedure order is not limited to any particular order of operation unless otherwise specified. In practice, distal bone portion 252 can be moved in any one or more planes prior and/or while moving in any one or more other planes. The clinician can adjust a position of distal bone portion 252 relative to proximal bone portion 250 in the frontal plane and/or sagittal plane prior to, after, and/or while adjusting the position in the transverse plane. Further, while example procedural steps have been described as being executed by instrument 100, a clinician may additionally or alternatively move distal bone portion 252 relative to proximal bone portion 250 in one or more planes without the aid of instrument 100 (e.g., by grasping distal bone portion 252 directly or by grasping a pin inserted into the bone portion and manipulating the positioning of the bone portion in one or planes).

After suitably moving distal bone portion 252 relative to proximal bone portion 250 in one or more planes, for example to correct a bunion deformity, the technique of FIG. 4 can involve temporarily fixating a moved position of the distal bone portion prior to permanent fixation (step 24 in FIG. 4). In some examples, one or more fixation wires are inserted through one or more corresponding fixation apertures of body 102 of instrument 100 to a position of distal bone portion 252 relative to instrument 100. The system may be configured such that the one or more fixation wires, when inserted into distal bone portion 252, provide a target indication aligned with a screw insertion trajectory defined by screw targeting guide 110 (FIG. 7B) for orienting and guiding insertion of one or more permanent fixation screws.

A screw insertion trajectory defined by screw targeting guide 110 can refer to an axis, path, or directional line along which a definitive fixation screw is intended to be advanced into bone. In some applications, screw targeting guide 110 may define one or more such screw insertion trajectories by virtue of one or more openings, guide surfaces, sleeves, passages, or other structural features that establish the intended position and orientation of a screw relative to instrument 100, proximal bone portion 250, distal bone portion 252, or another target anatomy. A screw inserted along a given screw insertion trajectory may pass through an opening defined by screw targeting guide 110 during insertion. In other uses, however, screw targeting guide 110 may be used to establish the screw insertion trajectory indirectly, for example by guiding placement of a pin, drill, awl, sleeve, cannula, pilot feature, or other intermediate guide structure that thereafter defines or preserves the intended trajectory for subsequent screw insertion.

Accordingly, in some applications, the definitive fixation screw may be advanced through screw targeting guide 110 itself along the screw insertion trajectory defined by screw targeting guide 110. In other applications, the screw insertion trajectory may first be established using screw targeting guide 110, after which screw targeting guide 110 and/or instrument 100 may be removed, repositioned, or no longer used for direct screw guidance while the definitive fixation screw is subsequently inserted along the previously established trajectory. Thus, screw targeting guide 110 may be configured to define, set, establish, and/or preserve a screw insertion trajectory independent of whether the definitive fixation screw physically passes through screw targeting guide 110 during final implantation.

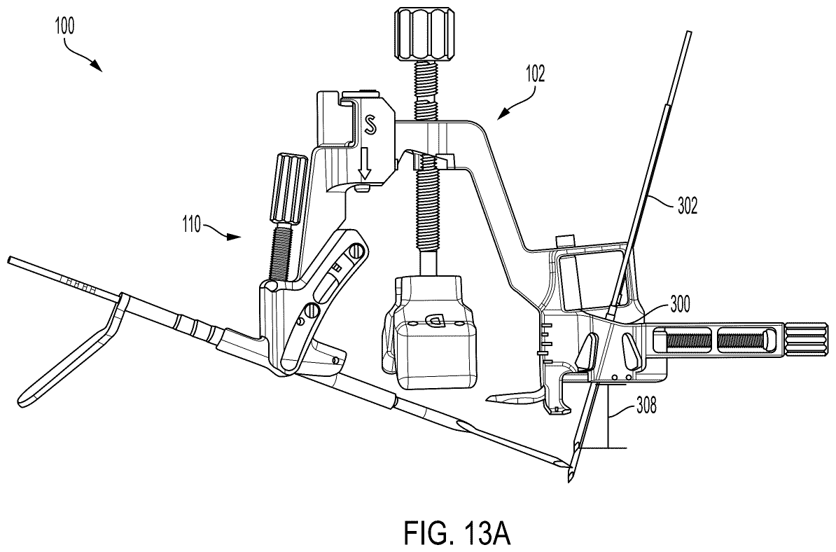

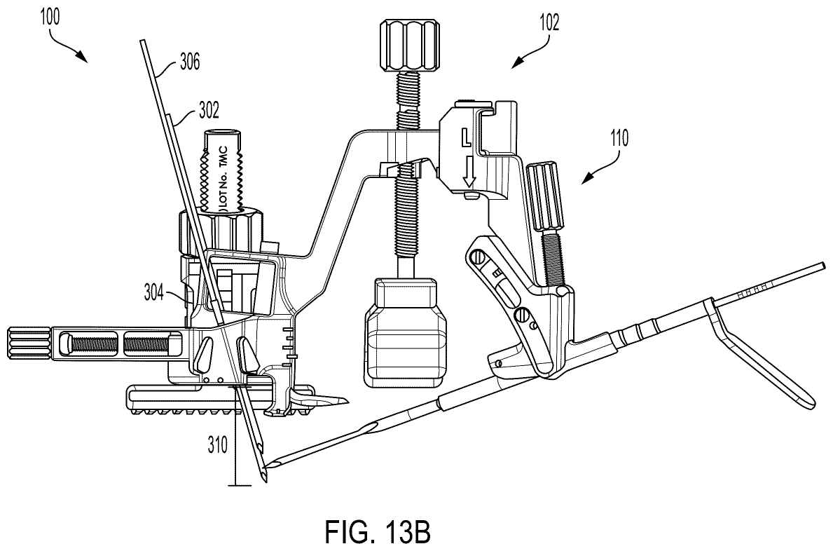

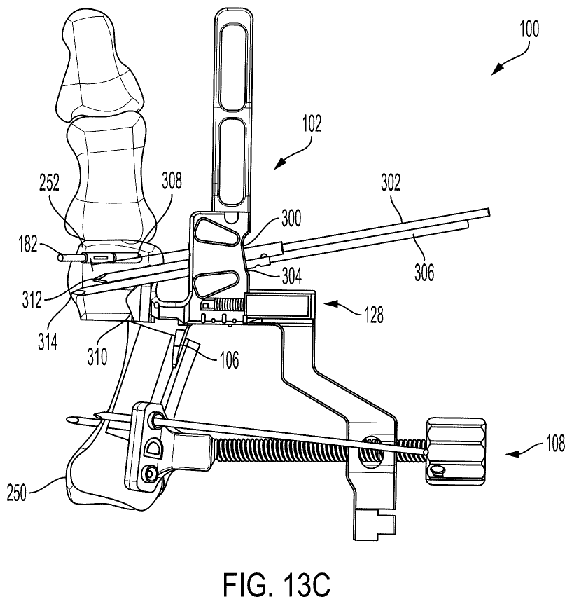

FIGS. 13A-13C (collectively “FIG. 13”) are different views of instrument 100 showing example fixation aperture and fixation wire arrangements that can be used to fixate distal bone portion 252 relative to the instrument. FIG. 13A is a dorsal-to-plantar view of instrument 100 (e.g., as the instrument would be positioned in use on a medial side of the bone) showing an example arrangement of features (with bone positioning device 160 detached from the device for purposes of illustration). FIG. 13B is a plantar-to-dorsal view of instrument 100 (e.g., as the instrument would be positioned in use on a medial side of the bone) showing an example arrangement of features. In addition, FIG. 13C is another dorsal-to-plantar view of instrument 100 shown engaged with proximal bone portion 250 and distal bone portion 252 (with bone positioning device 160 detached from the device for purposes of illustration).

As shown in the illustrated example of FIG. 13, body 102 of instrument 100 can define at least one fixation aperture 300 configured to guide a fixation wire 302 into the distal bone portion 252. In the illustrated arrangement, body 102 defines a first fixation aperture 300 configured to guide a first fixation wire 302 into distal bone portion 252 and a second fixation aperture 304 configured to guide a second fixation wire 306 into the distal bone portion. Body 102 can define more or fewer fixation apertures without departing from the scope of the disclosure.

Each fixation aperture 300, 304 defined by instrument 100 (e.g., body 102 of the instrument) can have a longitudinal length defining a longitudinal axis along which a fixation wire inserted through the fixation aperture is guided. Accordingly, the longitudinal axis of each fixation aperture 300, 304 may define a wire insertion trajectory that intersects distal bone portion 252. As a result, when fixation wire 302, 306 is inserted through a corresponding fixation aperture, the wire can pierce the cortical wall of distal bone portion 252 and be inserted into the bone portion. In practice, fixation aperture 300, 304 may overlie a portion of skin covering distal bone portion 252 and fixation wire 302, 306 may be inserted percutaneously through the skin.

In the example of FIG. 13, fixation apertures 300, 304 are located distally of intramedullary insertion body 106 and intramedullary insertion body translation mechanism 128 along the length of proximal bone portion 250 and distal bone portion 252 (e.g., when instrument 100 is positioned on the medial side of the bones during use). First fixation aperture 300 and second fixation aperture 304 may be positioned on different midline sides of body 152 (e.g., dorsal-to-plantar midline). For example, first fixation aperture 300 may be positioned dorsally of intramedullary insertion body 106 (e.g., when instrument 100 is positioned on the medial side of the bones during use) and/or second fixation aperture 304 may be positioned plantarly of intramedullary insertion body 106 (e.g., when instrument 100 is positioned on the medial side of the bones during use). First fixation aperture 300 may be positioned to insert first fixation wire 302 in a dorsal half of distal bone portion 252. Second fixation aperture 304 may be positioned to insert second fixation wire 306 and a plantar half of distal bone portion 252.

In either case, the wire insertion trajectory defined by fixation aperture 300, 304 may extend in the transverse plane without being skewed in the sagittal and/or frontal plane. For example, the wire insertion trajectory defined by fixation aperture 300, 304 may extend from a medial side of distal bone portion 252 toward a lateral side of the distal bone portion in the transverse plane without angling dorsally or plantarly in the sagittal plane moving from the medial to lateral side of the distal bone portion. This can be useful, for example, to consistently control the depth and positioning of fixation wire 302, 306 in distal bone portion 252 for subsequent visualization. That being said, in other configurations, the wire insertion trajectory defined by fixation aperture 300, 304 may be angled in the sagittal plane such that fixation wire 302, 306 extends dorsally or plantarly moving from the medial side of distal bone portion 252 toward the lateral side of the bone portion.

Instrument 100 and/or fixation wire 302, 306 may be configured so that the fixation wire provides a screw insertion indicating functionality once inserted into distal bone portion 252 in addition to or in lieu of functioning to fixate a position of the distal bone portion relative to instrument 100. For example, instrument 100 may be configured so there is a set and repeatable distance between body 102 of instrument 100 and the tip fixation wire 302, 306 (when the fixation wire is fully inserted into fixation aperture 300, 304). This distance and/or the position of the tip of the fixation wire relative to one or more screw insertion trajectories defined by screw targeting guide 110 can be set (e.g., during manufacture, design) and repeatable between uses of instrument 100. The clinician can identify the location of fixation wire 302, 306 once inserted into distal bone portion 252 to set screw targeting guide 110 and/or to select one or more screws to install for permanent fixation.

For example, as shown in FIG. 13, first fixation wire 302 may extend a first length 308 from body 102 of instrument 100. Second fixation wire 302 may extend a second length 310 from body 102 of instrument 100, where the second length is different than the first length (e.g., larger than the first length). When configured with additional fixation apertures and/or a system of additional wires, additional fixation wires (e.g., two, three, four, five, or more) may be provided that are each configured to extend a different length from the instrument. A clinician may select and insert through a fixation aperture one or fixation wires from the system of different wires to identify a target location in distal bone portion 252 for guiding one or more permanent fixation screws.

In some applications, a clinician need not rely on a system of multiple fixation wires having different predefined lengths to identify a desired location in distal bone portion 252. Instead, a single fixation wire 302 may be inserted through fixation aperture 300 and/or 304 and advanced into distal bone portion 252, and the position of the tip of the single fixation wire may be determined based on the extent to which the fixation wire has been advanced relative to instrument 100. For example, the clinician may measure, estimate, or otherwise determine a length of fixation wire extending from body 102 and/or extending into bone using fluoroscopic imaging, one or more depth markings on fixation wire 302, a scale associated with instrument 100, and/or another depth-indicating feature. Based on the identified depth and the known relationship between fixation aperture 300 and/or 304 and one or more screw insertion trajectories defined by screw targeting guide 110, the clinician may identify a target location for one or more permanent fixation screws, adjust screw targeting guide 110, and/or select one or more screws for implantation. Accordingly, location-identification functionality may be achieved using a single fixation wire 302 in addition to or instead of using multiple fixation wires having different extension lengths.

During a procedure, a clinician can insert first fixation wire 302 through first fixation aperture 300 and advance the wire laterally into distal bone portion 252. The tip of first fixation wire 302 can penetrate a cortical wall (e.g., medial cortical wall) of distal bone portion 252 and can continue advancing laterally toward an opposite cortical wall (e.g., lateral cortical wall). The clinician can continue advancing first fixation wire 302 through first fixation aperture 300 until a set depth indication. The depth indication may be a line or other visual indicator on first wire 302 that the clinician can insert first wire up to. Additionally or alternatively, the depth indication may be a physical stop, such as an enlarged collar on first wire 302 or other mechanical engagement feature, that physically prevents the clinician from advancing first wire 302 deeper into distal bone portion 252 beyond the depth indicated by the physical stop. Depending on the length 308 that first wire 302 extends from instrument 100 and the size of distal bone portion 252 for the particular patient being operated on, the tip of first wire 302 may be in the medullary canal and/or cancellous bone of distal bone portion 252 or may partially or fully project through the lateral cortical wall of the bone portion.

In some examples, the clinician inserts one or more additional fixation wires through the same fixation aperture or a different fixation aperture that are sized to be inserted to a different depth than first fixation wire 302. For example, the clinician can insert second fixation wire 306 through second fixation aperture 304 and advance the wire laterally into distal bone portion 252. The tip of second fixation wire 306 can penetrate a cortical wall (e.g., medial cortical wall) of distal bone portion 252 (optionally advancing through a hole already formed by the first fixation wire that was subsequently removed) and can continue advancing laterally toward an opposite cortical wall (e.g., lateral cortical wall). The clinician can continue advancing second fixation wire 306 through second fixation aperture 304 until a set depth indication. Again, the depth indication may be a line or other visual indicator on second wire 306 that the clinician can insert second wire up to. Additionally or alternatively, the depth indication may be a physical stop, such as an enlarged collar on second wire 306 or other mechanical engagement feature, that physically prevents the clinician from advancing second wire 306 deeper into distal bone portion 252 beyond the depth indicated by the physical stop. Depending on the length 310 that second wire 306 extends from instrument 100 and the size of distal bone portion 252 for the particular patient being operated on, the tip of second wire 306 may be in the medullary canal and/or cancellous bone of distal bone portion 252 or may partially or fully project through the lateral cortical wall of the bone portion.

With reference to FIG. 13C, first fixation wire 302 is illustrated as extending to a first target medial-to-lateral insertion location 312 (as identified by the tip of the wire) for guiding a drill and/or screw by screw targeting guide 110. Second fixation wire 306 is illustrated as extending to a second target medial-to-lateral insertion location 314 (as identified by the tip of the wire) for guiding a drill and/or screw by screw targeting guide 110). First target location 312 is more medial than second target location 314. One or more additional or different fixation wires that extend to a different medial-to-lateral depth can be inserted in distal bone portion 252.

The different target locations 312, 314 can indicate different targeting points that a drill and/or screw guided by screw targeting guide 110 can be advanced toward. For example, the different targeting points can indicate different locations in a medial-to-lateral direction across distal bone portion 252 that a drill and/or screw guided by screw targeting guide 110 can be targeted. The clinician can select one of the different target locations 312, 314 and use that target location for controlling screw targeting guide 110 and/or guiding one or more features using the screw targeting guide. For example, the clinician may select a target location 312, 314 that intersects with a lateral cortex of distal bone portion 252 without being located laterally beyond the lateral cortical wall of the bone. This can allow the clinician to place a screw through proximal bone portion 250 and into distal bone portion 252 with the tip of the screw engaged with the lateral cortical wall of the distal bone portion. The clinician may choose additional or different target locations depending on the anatomical characteristics and specific conditions of the procedure.

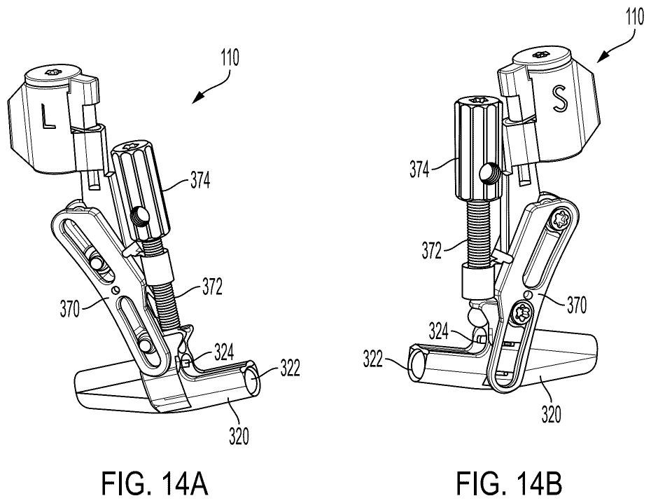

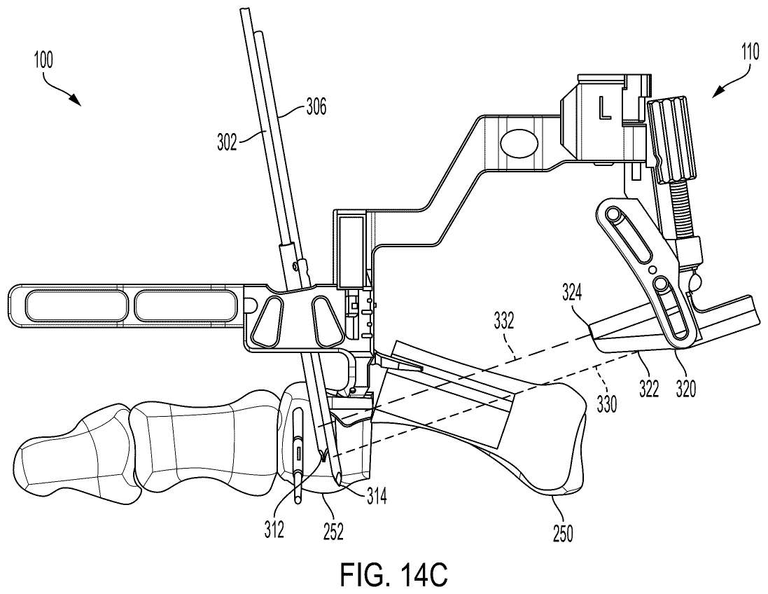

FIGS. 14A and 14B are different views of an example configuration of screw targeting guide 110 that can be used with instrument 100 according to the disclosure. Screw targeting guide 110 can have a guide body 320 having one or more openings each defining a screw insertion trajectory there through which, in the illustrated example, is shown as a first opening 322 defining a first screw insertion trajectory and a second opening 324 defining a second screw insertion trajectory. Each screw insertion trajectory can be defined by an axis extending lengthwise through the opening (e.g., parallel to the longitudinal axis of the portion of body 320 defining the opening). The screw insertion trajectory defined by first opening 322 and the screw insertion trajectory defined by second opening 324 can be parallel to each other to facilitate placement of the two parallel fixation screws. In use, a clinician can insert one or more instruments and/or implants through opening 322, 324, as will be described, to facilitate permanent fixation of proximal bone portion 252 distal bone portion 252 after alignment. While guide body 320 is configured with two openings, a screw targeting guide according to disclosure can have fewer guide openings or more guide openings (e.g., three, four, five or more) without departing from the scope of disclosure

Screw targeting guide 110 can be operatively connected to body 102 of instrument 100. In some configurations, screw targeting guide 110 is permanently connected to body 102 of instrument 100 (e.g., formed as a unitary component or formed as separate components permanently coupled together). In other configurations, screw targeting guide 110 is detachably connected to body 102 of instrument 100. When configured to be detachable from body 102, screw targeting guide 110 may be connected to body 102 of instrument 100 throughout the procedure or may be attached during the procedure when ready to utilize screw targeting guide 110. Accordingly, while example procedural steps have been illustrated and described above without screw targeting guide 110 being attached to instrument 100, it should be appreciated that the screw targeting guide may or may not be connected to the instrument when performing other procedure steps as described herein.

When configured to be detachably connected, a variety of different mechanical connection features can be used to interconnect screw targeting guide 110 to body 102 of instrument 100. For example, with reference to FIGS. 7A and 7B, screw targeting guide 110 and body 102 of instrument 100 may have complementary male and female connection features that allow the two components to be joined together. In the illustrated example, body 102 is illustrated as including a male connector 326 that is insertable into a complementary female receiving opening of screw targeting guide 110. Once assembled, screw targeting guide 110 can be frictionally retained to body 102 of instrument 100, or other mechanical attachment features (e.g., screw, clasp, lock) may be used to couple the screw targeting guide to body 102. Once assembled, screw targeting guide 110 may be positioned proximally of bone positioning device 108, intramedullary insertion body translation mechanism 128, and bone positioning device 160.

FIG. 14C is a dorsal-to-plantar view of instrument 100 with screw targeting guide 110 attached illustrating an example procedure step and arrangement of components. In the illustrated example, bone positioning devices 108 and 160 are shown removed from instrument 100 for purposes of clarity. As shown in this example, body 320 of screw targeting guide 110 can be connected to and/or extend from an arm that positions the body defining opening, 320, 322 proximally and/or laterally offset relative to a portion of the body from which the arm extends.

As shown in FIG. 14C, a first screw insertion trajectory 330 can be defined by first opening 322 that intersects proximal bone portion 250 and distal bone portion 252. Similarly, a second screw insertion trajectory 332 can be defined by second opening 324 that intersects proximal bone portion 250 and distal bone portion 252. Second screw insertion trajectory 332 can be parallel to first screw insertion trajectory 330. Each screw insertion trajectory may be a projection of a longitudinal axis defined by the lengthwise extent of first opening 322 and second opening 324, respectively.

Body 320 can be positioned by instrument 100 relative to proximal bone portion 250 and distal bone portion 252 such that each screw insertion trajectory defined by the body extends from a proximal-medial to distal-lateral direction. For example, one or both screw insertion trajectories 330, 332 may intersect a medial side of proximal bone portion 250 on a proximal-most half of the bone, such as proximal-most third of the bone. The target location for one or both screw insertion trajectories 330, 332 to intersect in distal bone portion 252 may be set by indicating wires 302, 306 and manipulation of instrument 100 to align at least one opening defined by body 320 with a target location 312 or 314 selected by the clinician.

The example technique of FIG. 4 can further include positioning screw insertion guide 110 to align with a target insertion location identified at distal bone portion 252 (step 24 in FIG. 4). For example, in some configurations, instrument 100 may be configured to guide a drill and/or screw along one or screw insertion trajectories defined by the instrument. In some configurations, the clinician is able to manipulate instrument 100 to adjust the orientation of body 320 and openings 322, 324 defined thereby to define any desired screw insertion trajectory and/or target locations 312, 314 relative to distal bone portion 252. In other configurations, instrument 100, and screw targeting guide 110, may provide a discrete number of screw insertion trajectories and/or target locations 312, 314 that the clinician can select from. For example, instrument 100, and screw targeting guide 110 may provide a discrete number of positions that body 320 can be set to relative to body 102 to provide a discrete number of target locations 312, 314 (e.g., terminal or destination points at the distal bone portion 252) a clinician can select a target using screw targeting guide 110. In different examples, the discrete number of target locations 312, 314 in distal bone portion 252 that may be selectable for targeting by screw targeting guide 110 may be two, three, four, five, six, or more. In some cases, the discrete number of targeting locations is two, three, or four (e.g., three) to limit the complexity of the system and prevent the clinician from being overwhelmed with choice.

Instrument 100 and/or screw targeting guide 110 can have a variety of different features that allow body 320 to be set at a discrete number of positions for targeting a discrete number of target locations 312, 314 in the medial-to-lateral and/or distal-to-proximal direction across distal bone portion 252. In some configurations, instrument 100 and/or screw targeting guide 110 is configured with an adjustable mechanical connection that controls the positioning of body 320 (e.g., in a medial-to-lateral direction when attached to body 102 and instrument 100 is positioned on a medial side of the bone portions) to one of a plurality of set offset distances. Manipulation of instrument 100 to select one of the plurality of discrete settings can align a screw insertion trajectory (e.g., the lateral-most screw insertion trajectory 330) defined by body 320 with a select one of the identified target locations 312, 314 identified by fixation wires 302, 306. The screw insertion trajectory can be aligned with the target location at the distal bone portion 252 in that the screw insertion trajectory intersects the target location.

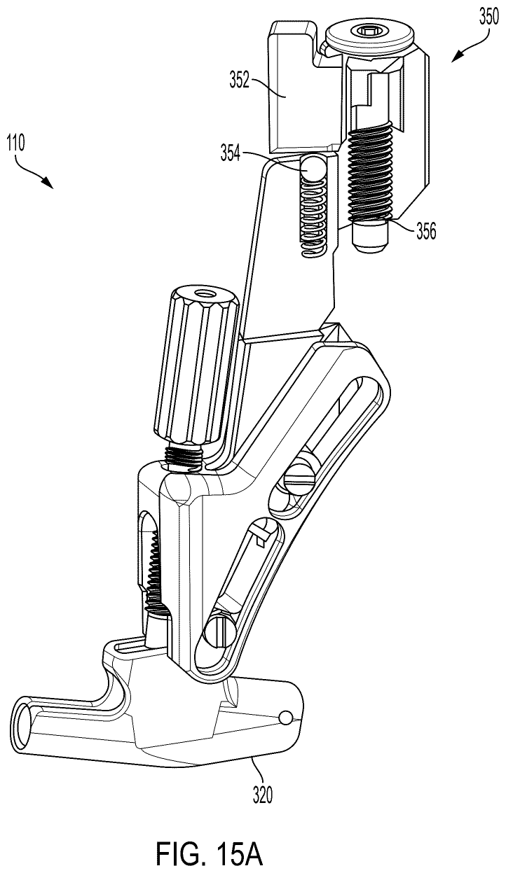

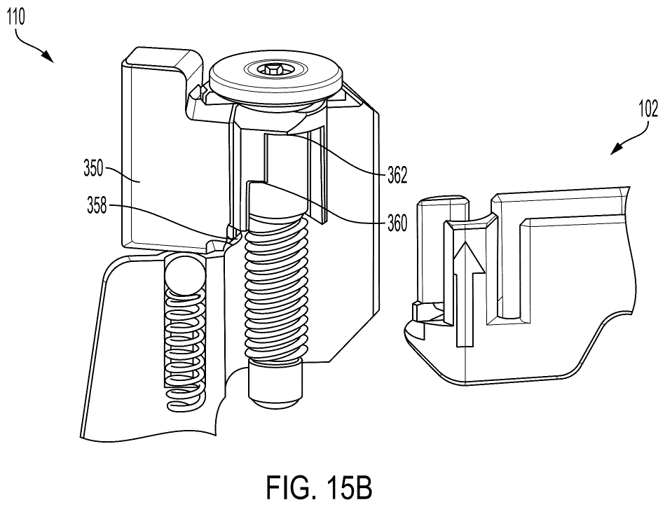

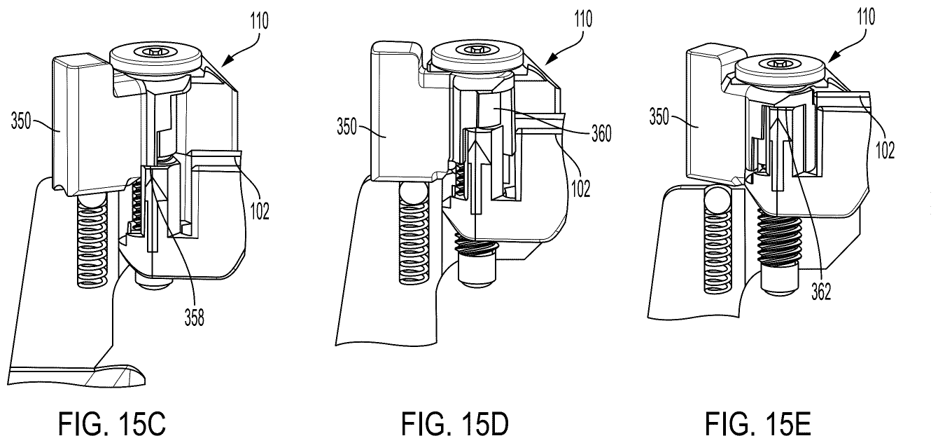

FIG. 15A as a partial cutaway of screw targeting guide 110 showing an example selector 350 that can be used by a clinician to select one of a plurality of identified target locations 312, 314 to be targeted. In the illustrate example, selector 350 is in the form of a toggle that includes a switch 352 movable between discrete positions, each of which corresponds to one of the plurality of discrete target locations that can be targeted at distal bone portion 252. Selector 350 can include a ball detent 354 that engages with switch 352. A thread 356 can be provided to draw screw targeting guide 110 tight to body 102 of instrument 100 once assembled.

FIG. 15B is an expanded partial cutaway of screw targeting guide 110 showing an example adjustable interface between the targeting guide and mating portion 326 of body 102 to which screw targeting guide 110 is configured to be operatively connected. In the illustrate example, screw targeting guide 110 is shown detached from but attachable to body 102 of instrument 100. As shown in this example, a plurality of discrete connection interfaces that are offset from each other are provided between screw targeting guide 110 and body 102 of instrument 100. In the illustrated example, the connection interface defined by screw targeting guide 110 includes a first interface 358, a second interface 360, and a third interface 362.

FIGS. 15C-15E illustrate screw targeting guide 110 interfacing with body 102 of instrument 100 at different discrete selectable meeting positions. When the connection interface of screw targeting guide 110 is mated with the corresponding mating portion 326 of body 102, body 320 carried by screw targeting guide 110 may be positioned closer to body 102 of instrument 100 when selector 350 is positioned to have first interface 358 contact a mating surface of body 102 (FIG. 15C). By contrast, screw targeting guide 110 may be positioned farther away from body 102 of instrument 100 when selector 350 is positioned to have second interface 360 contact a mating surface of body 102 (FIG. 15D). Screw targeting guide 110 can be positioned even farther away from body 102 of instrument 100 when selector 350 is positioned to have third interface 362 contact a mating surface of body 102 (FIG. 15E). As shown in FIGS. 15C-15E, rotating selector 352 different height faces can present different pairs of faces to the tower that on body 102 that receives and operatively coupled to screw targeting guide 110.

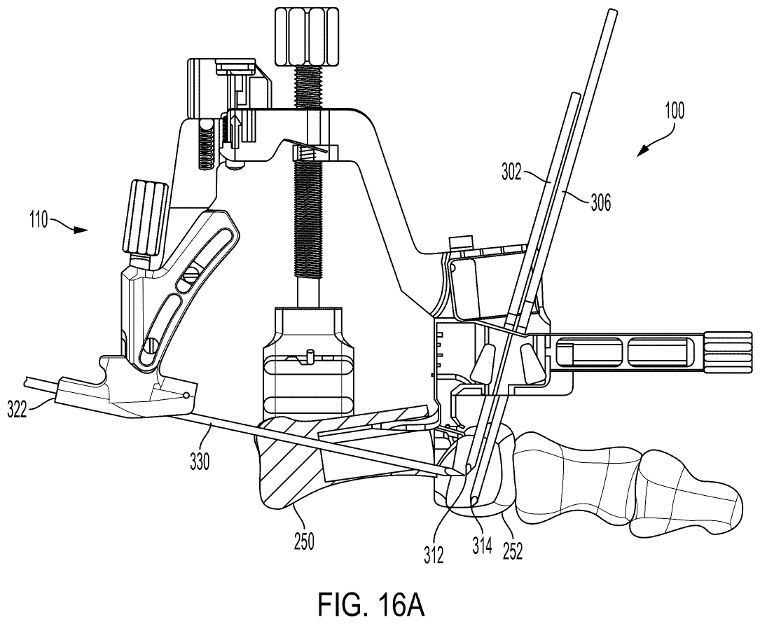

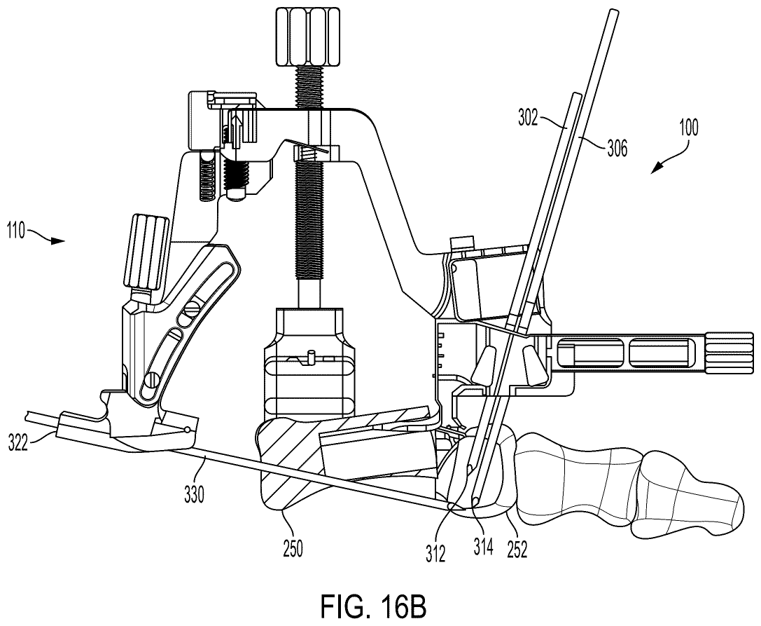

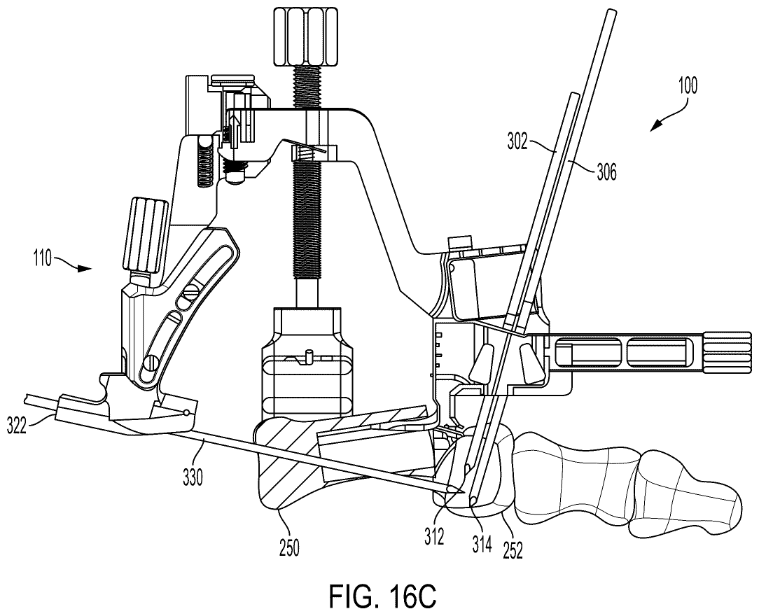

Each selectable position of screw targeting guide 110 can correspond to a different fixed target location 312, 314 at distal bone portion 252. Accordingly, the clinician can select one of the different discrete selectable screw targeting guide positions based on which target location at distal bone portion 252 the clinician would like to target to deliver an implant screw two. FIGS. 16A-16C show different example discrete target locations at distal bone portion 252 that can be selected to be targeted by screw targeting guide 110. FIG. 16A shows the lateral-most screw insertion trajectory defined by opening 322 of screw targeting guide 110 aligned with a first target location 312 at distal bone portion 252, which is a medial-most target location in the example. This can correspond to engaging first interface 358 with body 102 of instrument 100 via control of selector 350. FIG. 16B shows the lateral-most screw insertion trajectory defined by opening 322 of screw targeting guide 110 aligned with second target location 314 at distal bone portion 252, which is a lateral-most target location in the example. This can correspond to engaging third interface 362 with body 102 of instrument 100 via control of selector 350. FIG. 16C shows the lateral-most screw insertion trajectory defined by opening 322 of screw targeting guide 110 aligned with a third target location between the first and second target locations 312, 314, which is an intermediate medial-to-lateral target location in the example. This can correspond to engaging second interface 360 with body 102 of instrument 100 via control of selector 350.

After positioning screw insertion guide 110 to align with a target insertion location identified at distal bone portion 252, the technique of FIG. 4 can involve selecting a screw insertion trajectory entry point relative to proximal bone portion 250 (step 26 in FIG. 4). In some configurations, the screw insertion trajectory defined by body 320 is fixed relative to the selected target insertion location and cannot be modified. In other configurations, the screw insertion trajectory defined by body 320 (e.g., opening 322 of body 320) is adjustable about the selected target insertion location to control the location where the screw insertion trajectory intersects proximal bone portion 250.

For example, with further reference to FIGS. 14A and 14B, body 320 is illustrated as being connected to and/or carried by arm 370 and an adjustable shaft 372. In some configurations, body 320 is movable along an arc defined by arm 370 by actuating shaft 372. Shaft 372 may be a threaded shaft rotation of which causes body 320 to rotate along an arc, with the center of rotation about which body 320 rotates being the target insertion location at distal bone portion 252 selected to be targeted. In some examples, screw targeting guide 110 includes an actuator 374 (e.g., rotatable knob, screw drive) that a clinician can engage to control the position of body 320 about the arc. In various examples, body 320 may be connected via a threaded rod, rack and pinion, ratchet, and/or other mechanical linkage that allows the position of the body to be moved.

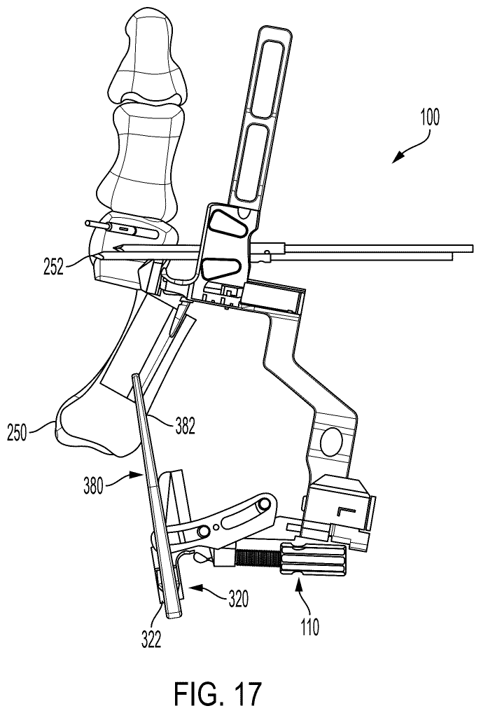

In some applications, the clinician engages an entry point identification tool with screw targeting guide 110 to visualize the screw insertion trajectory defined by the screw targeting guide to help set the desired insertion trajectory entry point. FIG. 17 is a dorsal-to-planar view showing an example entry point identification tool 380 that can be engaged with screw targeting guide 110 to visualize a screw insertion trajectory. Entry point identification tool 380 may be positioned in opening 322 of body 320 and extend parallel to the screw trajectory defined by the opening. In some examples, entry point identification tool 380 may be radiopaque to be visualizable under imaging (e.g., fluoroscopy). As shown in FIG. 17, entry point identification tool 380 may extend from opening 322 at least to the medial side of proximal bone portion 250 to visually indicate an intersection location 382 where the screw insertion trajectory will enter the medial side of the proximal bone portion.

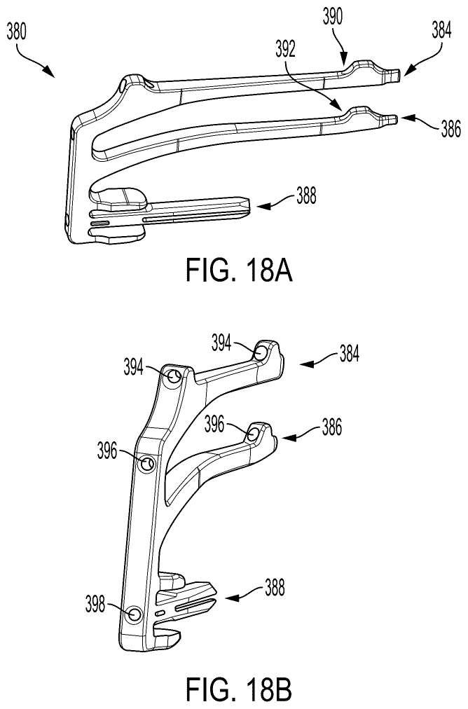

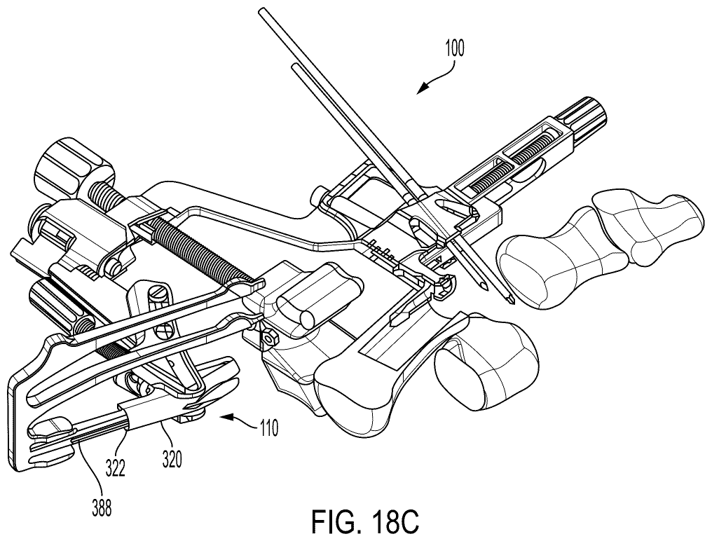

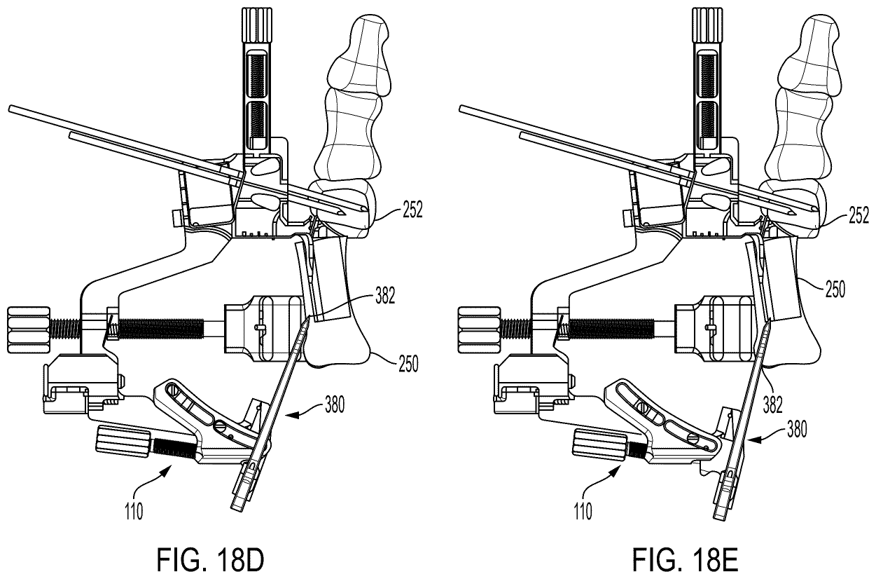

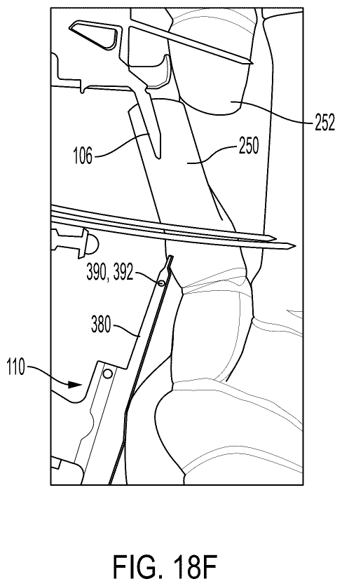

FIGS. 18A and 18B are different perspective views illustrating an example configuration of an entry point identification tool 380 that may be used according to disclosure. In this example, entry point identification tool 380 includes a first longitudinal arm 384 and a second longitudinal arm 386 parallel to the first longitudinal arm. The entry point identification tool 380 also includes a coupling feature 388, which is illustrated as a stem insertable into opening 322 of screw targeting guide 110. The one or more longitudinal arms which, in the illustrated configuration, are shown as first and second arms 384, 386, may be offset from coupling feature 388 to allow the arms to extend over a dorsal surface of the proximal bone portion 250 and/or under a plantar surface of the bone portion while the instrument is still engaged with screw targeting guide 110. In this way, the longitudinal arms can extend parallel to this screw insertion trajectory defined by screw targeting guide 110 without intersecting the bone portions through which the actual screw insertion trajectory extends.

In some configurations, first and second arms 384, 386 include one or more pairs of aligned holes 390, 392. Configuring the arms with aligned hole pairs can be useful for visualization during fluoroscopic imaging. When taking a dorsal-plantar fluoroscopic image with entry point identification tool 380 engaged, the aligned hole pairs will appear as a single aperture if the foot is square and aligned in the image. By contrast, if the portion of the foot being imaged is canted or off axis, the pair of holes in first and second arms 384, 386 may not appear fully aligned together or may not appear aligned at all. This can provide a visual indication to the clinician about the accuracy of the image which can be beneficial to ensure that the clinician is accurately viewing the location of any targeting wires inserted into distal bone portion 252. FIG. 18F is a dorsal-plantar fluoroscopic image showing entry point identification tool 380 positioned relative to proximal bone portion 250. The image shows a unitary hole, indicating that holes 390, 392 on the parallel and offset longitudinal arms are aligned and therefore the portion of the foot being imaged is accurately aligned in the plane being imaged.

In some configurations, entry point identification tool 380 additionally or alternatively includes one or horizontally extending apertures 394, 396, 398 through which a wire can be inserted. For example, a clinician may desire to guide a radiopaque wire parallel to and extending beyond first longitudinal arm 384 by guiding the wire through apertures 394 and/or guide a radiopaque wire parallel to and extending beyond second longitudinal arm 386 by guiding the wire through apertures 396. This can provide a visualization aid under imaging. Additionally or alternatively, the clinician may desire to guide a wire along the screw insertion trajectory by guiding the wire through aperture 398 to facilitate provisional fixation. For example,

FIG. 18C is a perspective illustration showing entry point identification tool 380 engaged with screw targeting guide 110 by advancing coupling feature 388 into opening 322. FIGS. 18D and 18E illustrate different example insertion trajectory entry point 382 at the medial side of proximal bone portion 250 that may be controllably targeted via actuation of actuator 274 on screw targeting guide 110. As shown, the screw insertion trajectory as indicated by entry point identification tool 380 can be rotated about an arc centered on the selected target location at distal bone portion 252. The clinician may set screw targeting guide 110 so the insertion trajectory entry point 382 is at the medial side of proximal bone portion 250 (e.g., as opposed to the proximal end face of the bone) and is not too steep of an angle, which may cause skiving.

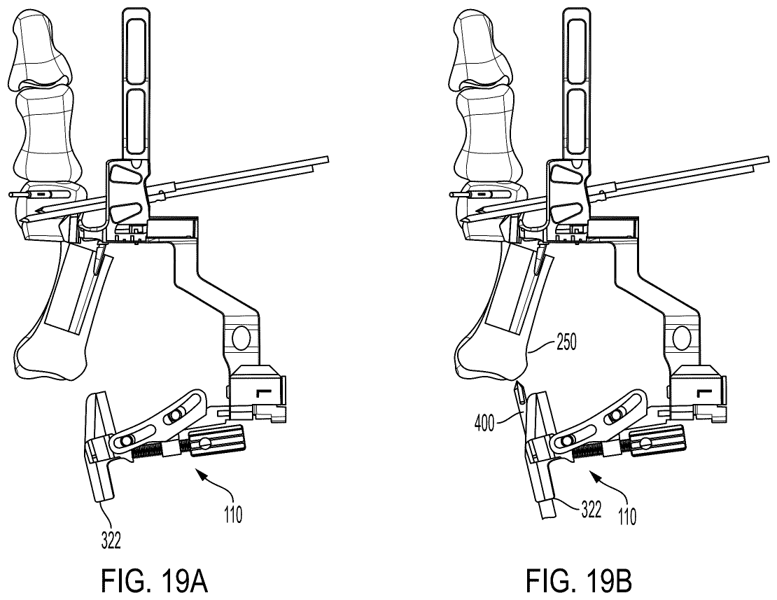

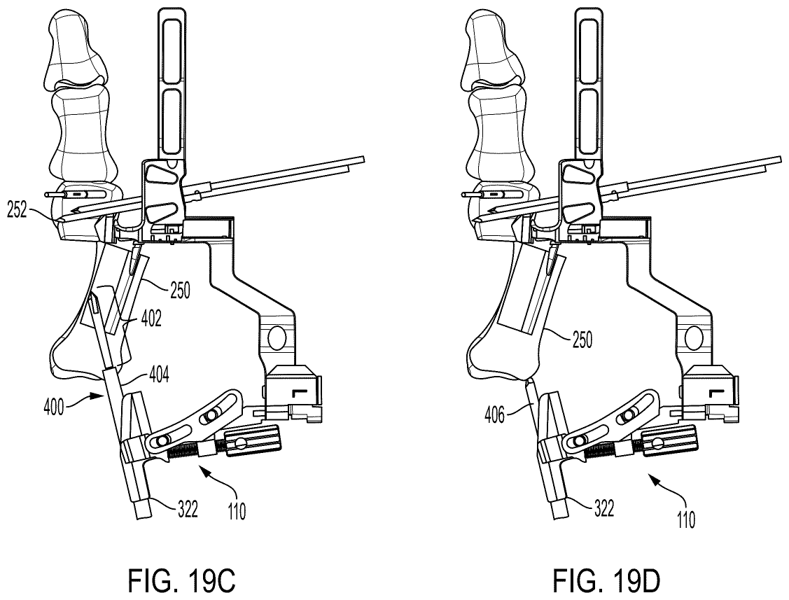

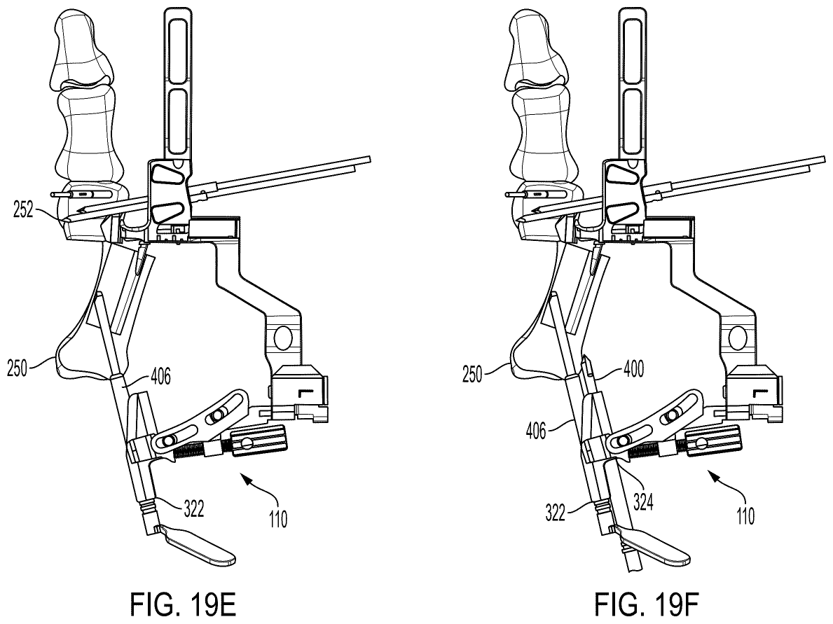

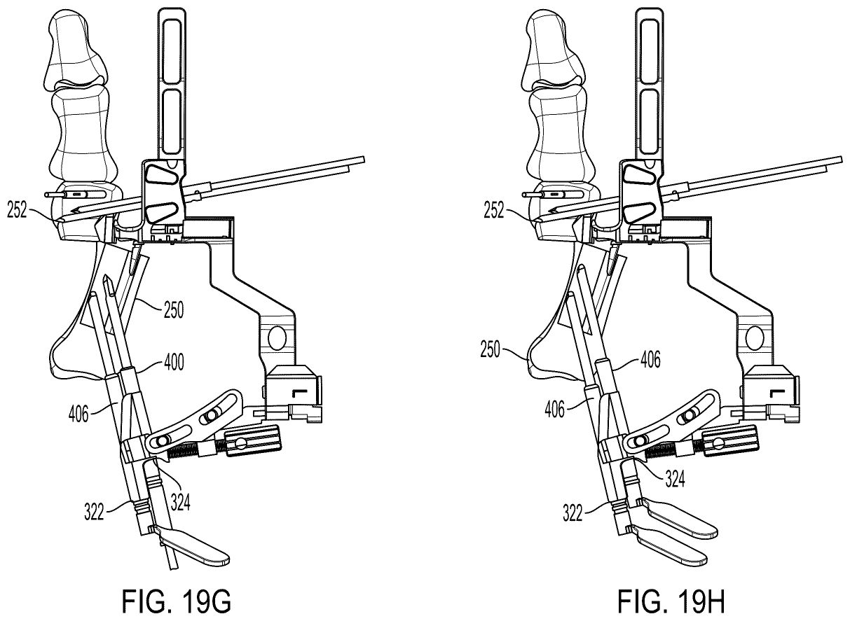

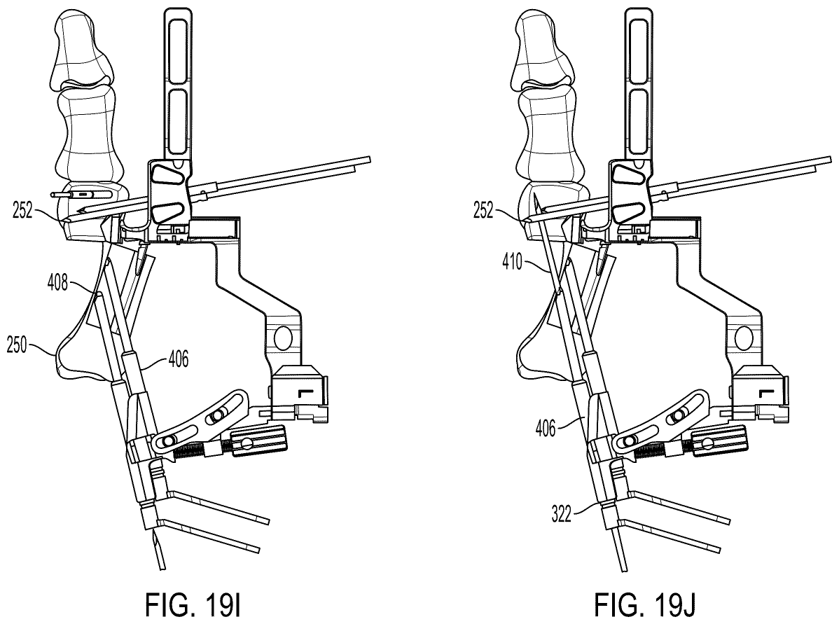

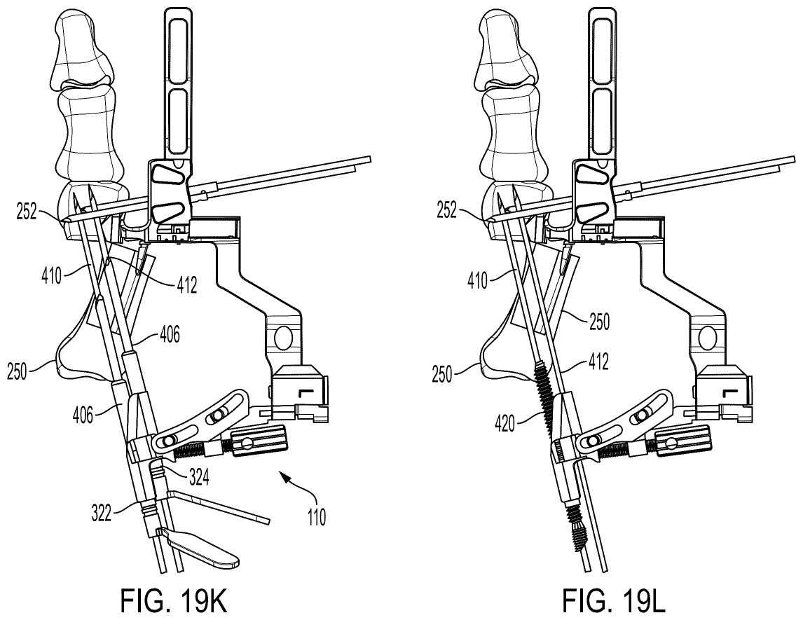

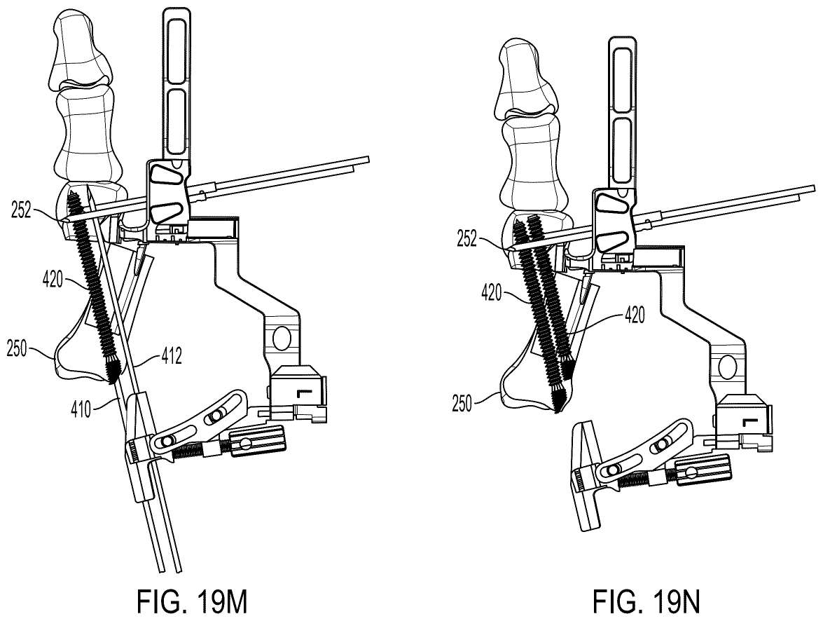

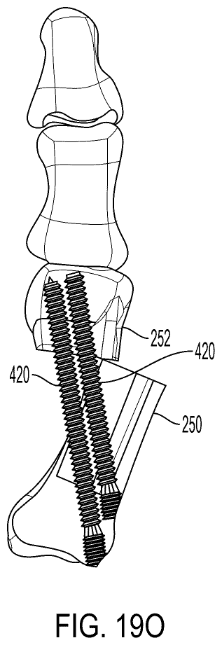

The example technique of FIG. 4 further involves inserting one or permanent screws through proximal bone portion 250 and into distal bone portion 252 along one or more screw insertion trajectories defined by screw targeting guide 110 (step 30 in FIG. 4). FIGS. 19A-19O illustrate example procedure steps that can be used to install screw fixation using instruments and techniques according to disclosure.

With reference to FIG. 19A, entry point identification tool 380 can be removed from opening 322 of screw targeting guide 110 to provide an open lumen for guiding one or more additional features. As shown in FIG. 19B, a drill 400 can be introduced into first opening 322 of screw targeting guide 100 and advanced along the screw insertion trajectory defined by the opening. Drill 400 may be any cutting instrument that forms an opening through bone for subsequent guiding insertion of a screw. Drill 400 may be a sharpened puncturing a wire, a drill bit attached to a hand-operated powered driver configured to rotate the drill bit, and/or other cutting instrument.

In various configurations, drill 400 may be cannulated or non-cannulated. For example, when drill 400 is cannulated, a k-wire may first be advanced through opening 322 of screw targeting guide 110 so that the k-wire defines an intended trajectory, and drill 400 may then be advanced over and along the k-wire to align drill 400 relative to the anatomy and/or relative to screw targeting guide 110. In other applications, drill 400 may be aligned and advanced along an axis defined by opening 322 without initially placing a k-wire through the opening, such that opening 322 and the associated guide features establish the intended trajectory for drill 400 and a k-wire is inserted after forming a drilled opening and removing drill 400. In still other applications, drill 400 may be used without utilizing a k-wire. Accordingly, the disclosed systems may employ wire-guided, wire-assisted, wire-first, drill-first, or non-wire techniques depending on the particular configuration and surgical workflow.

Regardless of whether a wire, drill, or another bone-penetrating instrument is used as the initial instrument to penetrate the bone, the instrument may be inserted percutaneously rather than through a primary incision that provides direct access to the location at which the metatarsal is cut. For example, the instrument may be introduced through the skin of the patient via a stab incision or other relatively small percutaneous opening. In some procedures, the instrument may be sufficiently sharp or otherwise configured to be advanced in a self-penetrating manner through the skin and into the underlying tissue. In other procedures, an initial skin incision or puncture may be created first, after which the wire, drill, or other instrument is advanced through the percutaneous access site toward the metatarsal. Accordingly, the disclosed techniques may employ percutaneous access independent of the particular type or sequence of bone-penetrating instrument used.

In some applications, a skin incision and/or soft tissue incision may be formed by guiding a cutting instrument through opening 322 of screw targeting guide 110 and advancing the cutting instrument axially into the skin and underlying soft tissue. For example, opening 322 may constrain the position and orientation of the cutting instrument such that the cutting instrument is directed along an intended access path toward bone. The clinician may advance the cutting instrument using an axial or plunging motion to pierce and cut through the skin and/or soft tissue, and additionally or alternatively may use a sweeping, rocking, or other lateralized cutting motion to enlarge or shape the incision as the cutting instrument progresses. In some procedures, the cutting instrument may be advanced only through the skin. In other procedures, the cutting instrument may be advanced farther through the soft tissue, for example down to bone or to a location adjacent the metatarsal. By guiding the cutting instrument through opening 322, the resulting incision may be positioned and aligned with the trajectory of one or more subsequent instruments and/or implants introduced through opening 322, which can facilitate more accurate percutaneous access and reduce the need for separate freehand localization.

In some procedures, where screw targeting guide 110 includes multiple openings 322, a cutting instrument may be sequentially advanced through each opening 322 to form separate skin incisions and/or soft tissue incisions associated with the respective openings 322. The resulting incisions may be spaced apart from one another such that a region of uncut skin remains between adjacent incisions. Such separated access sites may correspond to respective implant trajectories, drilling trajectories, fixation locations, or other instrument paths defined by the instrument. In other embodiments, the clinician may instead form a single larger incision that is accessible to multiple openings 322 of screw targeting guide 110, such that multiple instruments and/or implants can be introduced through the larger incision while still being guided by different openings 322. Additionally or alternatively, the clinician may form the skin incision or soft tissue incision without guiding the cutting instrument through opening 322, for example by creating the incision freehand before positioning screw targeting guide 110 or after screw targeting guide 110 has been positioned. Accordingly, the disclosed systems may employ guided incision formation, freehand incision formation, multiple discrete incisions, and/or a common larger access incision depending on the procedural workflow and anatomical considerations.

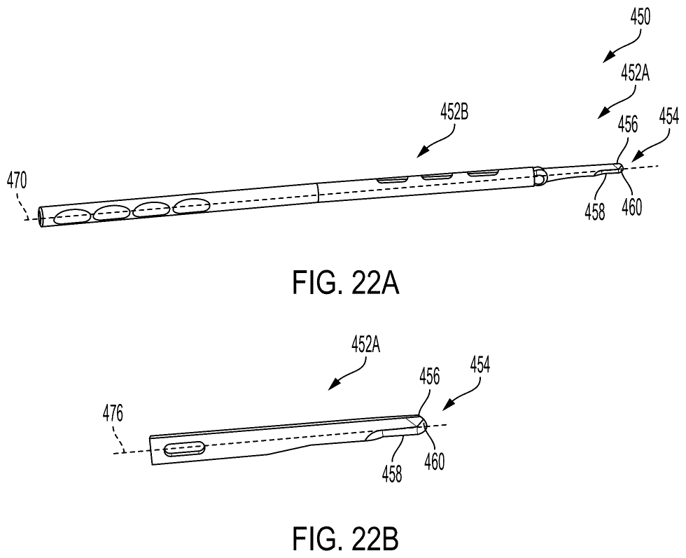

FIGS. 22A and 22B illustrate an example cutting instrument 450 that may be used to form an incision through the skin and/or soft tissue. As shown in FIG. 22A, cutting instrument 450 may include a blade 452A extending from or otherwise coupled to a handle 452B. FIG. 22B illustrates blade 452A separated from handle 452B, such that blade 452A and handle 452B together can define a replaceable blade assembly in which a used blade 452A may be removed and discarded while handle 452B is retained and reused. In other configurations, however, blade 452A and handle 452B may be formed as a unitary one-piece structure, for example machined from a single piece of metal, or may be formed as a multi-material structure such as a metal blade overmolded with a polymeric handle.

In some implementations, blade 452A may define a tip 454 having a rounded or pointed distal profile with cutting structure extending around the distal end of the blade rather than being limited to only one side of the blade. For example, tip 454 may include a dorsal cutting surface 456, a plantar cutting surface 458, and a distal cutting surface 460 extending between dorsal cutting surface 456 and plantar cutting surface 458 such that the cutting edge wraps around the distal-most rounded portion of tip 454. As a result, when blade 452A is advanced into tissue, tip 454 may cut tissue on both an upper side and a lower side of the blade, for example on both the dorsal side and the plantar side of the blade, rather than cutting predominantly on only one side as with certain conventional blade configurations. This wrapped cutting geometry can facilitate penetration and incision formation during axial advancement of cutting instrument 450 through the skin and/or soft tissue and can improve formation of an access path aligned with a desired instrument trajectory.

In some examples, blade 452A may be offset relative to handle 452B such that tip 454 is positioned to reach a desired tissue entry location more accurately when cutting instrument 450 is advanced toward the foot. For example, the offset arrangement can help place tip 454 slightly farther dorsally than would otherwise occur if blade 452A were centered on the handle axis, which may be beneficial in procedural contexts in which a subsequently introduced instrument, harpoon, sleeve, or other component tends to skive or translate distally along the bone or soft tissue during advancement. By initially forming the incision at a slightly more dorsal location, cutting instrument 450 can help compensate for such skiving behavior so that the subsequently inserted instrument reaches the intended anatomical location. In this manner, the offset geometry of blade 452A may improve the consistency and accuracy of percutaneous access site formation.

In some embodiments, tip 454 and/or one or more cutting surfaces of blade 452A may be positioned offset dorsally relative to a longitudinal axis 470 bisecting cutting instrument 450 as a whole. Such offset may be achieved in various ways. For example, blade 452A may be mounted to handle 452B in an offset relationship such that blade 452A is displaced dorsally relative to handle 452B and relative to longitudinal axis 470. Additionally or alternatively, blade 452A itself may include a bend, step, jog, or other transition feature 476 such that tip 454 is dorsally offset relative to a longitudinal axis 476 bisecting blade 452A along a more distal portion thereof. In some examples, the step or other offset feature 474 may cause dorsal cutting surface 456, plantar cutting surface 458, distal cutting surface 460, and/or tip 454 to be generally positioned more dorsally relative to axis 474 than proximally or centered along the axis. Accordingly, the desired entry-point compensation may be produced by offsetting blade 452A relative to handle 452B, by configuring blade 452A itself with an offset tip geometry, or by a combination thereof.

In some applications, cutting instrument 450 may be used by advancing blade 452A through opening 322 of screw targeting guide 110 such that opening 322 constrains the trajectory of blade 452A as tip 454 enters the skin and underlying soft tissue. The wrapped cutting geometry of tip 454 may facilitate formation of an incision during plunging advancement and may also assist cutting during sweeping or other secondary motion of cutting instrument 450. Although FIGS. 22A and 22B illustrate one example configuration, other blade shapes, handle geometries, attachment interfaces, and cutting edge arrangements may be employed while still providing a cutting instrument configured to create a percutaneous access incision aligned with a subsequent instrument and/or implant path.

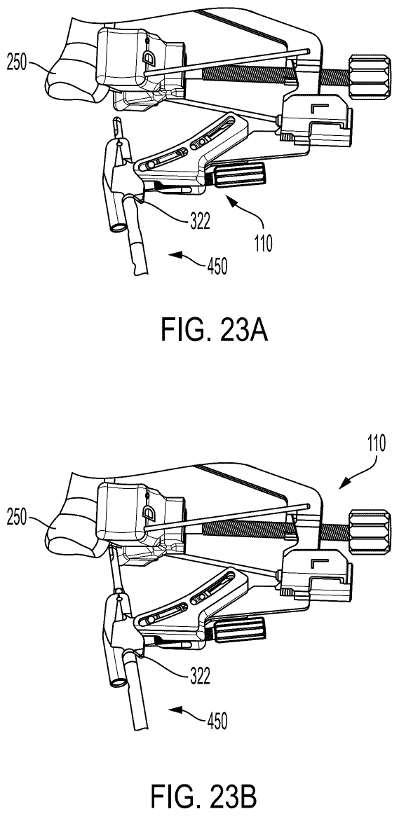

FIGS. 23A and 23B illustrate example use of cutting instrument 450 to form an incision through the skin and/or soft tissue by advancing cutting instrument 450 through opening 322 after the targeter has been adjusted to a desired entry point. In some examples, blade 452A may be mounted to handle 452B prior to use and then be inserted through one or more openings 322 to create one or more poke-hole incisions. Where screw targeting guide 110 includes multiple openings 322, the clinician may sequentially insert cutting instrument 450 through the respective openings 322 to form separate incisions corresponding to the respective subsequent instrument paths. By passing cutting instrument 450 through opening 322, the location and orientation of the resulting incision may be coordinated with the trajectory defined by screw targeting guide 110 for later-introduced instruments, sleeves, drills, wires, implants, or combinations thereof.

The offset geometry of blade 452A and/or tip 454 may bias the incision distally in the illustrated orientation. Such distal bias may be advantageous where one or more subsequently introduced instruments have a tendency to skive distally during advancement, such that the initially formed incision better corresponds to the effective entry location of the subsequently introduced instruments after such skiving occurs. In some procedures, a larger incision may be desired than that produced by a single insertion of cutting instrument 450. In such cases, cutting instrument 450 may first be advanced through opening 322 in a first orientation to form an initial incision, then withdrawn, rotated approximately 180 degrees, and reinserted through the same opening 322 to enlarge the incision, for example to create a double-width incision or another enlarged access opening.

In some applications, the wrapped cutting geometry of blade 452A may facilitate incision formation across a range of skin contours and approach angles. For example, because tip 454 may include cutting structure on multiple sides of the distal tip region, cutting instrument 450 may cut tissue during advancement even where the skin presents folds, uneven contour, or an oblique local surface orientation relative to the instrument path. This may reduce the tendency of the blade to snag prematurely or cut asymmetrically as compared with certain single-sided blade configurations. Similarly, where multiple poke-hole incisions are formed, the multi-sided cutting configuration may provide more consistent cutting performance during each pass, including passes that might otherwise be disadvantaged by blade orientation relative to the skin surface or insertion direction.

In some applications, because the angle at which cutting instrument 450 enters the skin can vary significantly depending on anatomy, skin contour, soft tissue tension, instrument orientation, and procedural approach, it may be difficult to reliably position a cutting edge at the intended entry point using a blade configuration sharpened on only one side. By contrast, where blade 452A includes tip 454 sharpened around substantially the entire distal tip region, such as along dorsal cutting surface 456, plantar cutting surface 458, and distal cutting surface 460, cutting may occur in a more consistent manner regardless of the particular approach angle of cutting instrument 450 relative to the skin. As a result, the full-tip cutting configuration can reduce variability associated with blade orientation and entry angle and can provide a more predictable incision at the intended access location. Additionally, when blade 452A is plunged distally to a depth adjacent bone, the circumferentially cutting tip 454 can more effectively release skin and soft tissue along the intended access path, thereby facilitating smoother advancement of subsequently introduced instruments. This can be advantageous relative to a freehand poke-hole incision, which may be too shallow, misaligned, or formed at a suboptimal angle and therefore may provide less reliable access for later procedural steps.

Returning to FIG. 19, with reference to FIG. 19C, drill 400 can be advanced through the medial cortical wall of proximal bone portion 250 and the tip of the drill advanced laterally and/or distally along the defined screw insertion trajectory up to the lateral cortical wall of proximal bone portion 250. In some examples the tip of drill 400 is advanced to cut partially but not fully into the lateral cortical wall of proximal bone portion 250. In other examples, the tip of drill 400 is advanced up to but without cutting into the lateral cortical wall of proximal bone portion 250. In either case, drill 400 may not penetrate through the lateral cortical wall of proximal bone portion 250. In other examples, drill 400 may be advanced beyond proximal bone portion 250 and into distal bone portion 252, for example prior to insertion of a k-wire and/or instead of inserting a k-wire. In such examples, drill 400 itself may establish the intended trajectory through proximal bone portion 250 and into distal bone portion 252 for a subsequent implant and/or other instrument.

In some configurations, such as is illustrated, drill 400 defines multiple diameters along its length including a first region 402 having a first diameter and a second region 404 having a second diameter larger than the first region. A shoulder or other size interface may be provided between the two regions. The shoulder or interface may be positioned along the length of drill 400 at a location that allows the enlarged portion of the drill to enter proximal bone portion 250 while drill 400 is still advancing through the proximal bone portion without penetrating the lateral cortical wall. Configuring drill with a shoulder and/or enlarged region can be beneficial to increase the stiffness of the drill and decrease skiving during drilling, helping to ensure accurate targeting during drilling.

With reference to FIG. 19D, drill 400 can be withdrawn from proximal bone portion 250 to leave a hole formed by the drill. A sleeve 406 can then be guided through opening 322 into the hole previously formed by drill 400. Sleeve 406 may be advanced until it contacts the lateral wall of proximal bone portion 250, as illustrated in FIG. 19E. Sleeve 406 may define a lumen sized to pass a subsequent drill instrument. Sleeve 406 can help maintain the orientation of the whole initially formed by drill 400 and also stabilize the interconnection between instrument 100 and proximal bone portion 250, helping to increase the stiffness and cohesiveness to help ensure accuracy during further drilling.

With sleeve 406 inserted into proximal bone portion 250 following the opening formed by drill 400, screw insertion trajectories defined by one or more additional openings of screw targeting guide to 110 can be drilled and sleeved. For example, drill 400 can be advanced through opening 324 extending parallel to opening 322, penetrating the medial cortical wall of proximal bone portion 250 and advancing up to but not through the lateral cortical wall of the bone portion, as illustrated in FIGS. 19F and 19G. Thereafter, drill 400 can be retracted and another sleeve 406 inserted into the opening formed by drill 400, as discussed above with respect to drilling through opening 322, as illustrated in FIG. 19H.

FIG. 19I illustrates sleeves 406 inserted through openings 322 and 324 and position in proximal bone portion 250. The distal tips of sleeves 406 are advanced up to but not through the lateral cortical wall of proximal bone portion 250. In this example, each sleeve 406 includes an angled or beveled tip 408 having a slope selected to generally conform to the slope of the lateral cortical wall.

With sleeves 406 inserted, the clinician can insert a first wire 410 through opening 322 of screw targeting guide 110 and, more particularly, through the cannulated sleeve 406 extending through the opening. Wire 410 can penetrate the lateral cortical wall of proximal bone portion 250 and enter the distal bone portion 252 (e.g., through the cut end face of the distal bone portion), as shown in FIG. 19J. The clinician can insert a second wire 412 through opening 324 of screw targeting guide 110 and, more particularly, through the cannulated sleeve 406 extending through the opening. Wire 412 can penetrate the lateral cortical wall of proximal bone portion 250 and enter the distal bone portion 252 (e.g., through the cut end face of the distal bone portion), as shown in FIG. 19K.

With first and second wires 410, 412 inserted extending through proximal bone portion 250 and into distal bone portion 252 along the preestablished screw insertion trajectory, the clinician can remove sleeves 406 from screw targeting guide of 110 leaving the wires in place. The clinician can then select a cannulated screw having desired length and characteristics to advance over each wire. The length of each wire 410, 412 inserted into proximal bone portion 250 and distal bone portion 252 may include a depth indicating in line and/or other measurement feature may be used to determine the length of screw to use based on the depth of the wire. In some configurations, the screw selected to be inserted over each wire 410, 412 includes a first threaded region configured to be threateningly engaged in proximal bone portion 250 and a second threaded region configured to be threateningly engaged in the distal bone portion 252. The screw may have a continuous thread along an entirety of its length or one or more discrete regions of threading separated by regions that are unthreaded and/or regions having a different configuration of threading than one or more other adjacent regions. Example screw configurations that may be used, but are not required and therefore the disclosure is not limited in this respect, are described in United Stated Provisional Patent Application No. 63/649,208, filed May 17, 2024, and titled “ORTHOPEDIC SCREW AND DRIVER SYSTEM FOR MINIMALLY INVASIVE METATARSAL CORRECTION PROCEDURE,” the entire contents of which are incorporated herein by reference.

FIG. 19L illustrates a first cannulated screw 420 partially advanced over first wire 410. FIG. 19M illustrates screw 420 fully inserted into proximal bone portion 250 and distal bone portion 252 along the screw insertion trajectory set by instrument 100. Screw 420 may include a chamfered end 422 having an angle configured to align with the angle of the medial wall of proximal bone portion 250. After insertion, first wire 410 can be withdrawn, leaving screw 420 in the bone portions for permanent fixation. A second screw 420 can be inserted over second wire 412 and the wire subsequently withdrawn following the same process as discussed, as shown in FIG. 19N. Instrument 100 can subsequently be decoupled leaving screws 420 inserted into proximal bone portion 250 and distal bone portion 252 across the osteotomy between the two bone portions, thereby stabilizing the bone portions for permanent fixation allowing bone in growth and fusion of the bone portions.

Any of the instruments, devices, and/or implants described herein can be designed and constructed with patient-specific sizing and/or characteristics (e.g., one or more characteristics configured to interface with patient-specific anatomical attributes). In these examples, the anatomical characteristics (e.g., size and/or shape) of at least a portion of the patient's foot undergoing the procedure can be determined prior to performing the surgical procedure. The patient's foot may be imaged to provide data indicative of the size and structure of the patient's foot. A computational model representative of the patient's foot may then be generated and one or more of the instruments and/or implants to be used during the procedure sized, shaped, and/or otherwise configured to the specific anatomical characteristics of the foot of the patient undergoing the procedure. The instruments and/or implants can then be manufactured to provide one or more patient-specific components that are then used during the subsequent surgical procedure. For example, the instruments and/or implants may have one or more surface features size and shape indexed to corresponding anatomical location(s) of the patient's bone where the features can be positioned.

Various examples have been described. These and other examples are within the scope of the following claims.

1. A bunion treatment method comprising:
cutting a metatarsal bone of a foot into a distal metatarsal portion and a proximal metatarsal portion;
applying a medial-directed pulling force to a distal end of the proximal metatarsal portion with an instrument engaged with the proximal metatarsal portion and applying a lateral-directed pushing force to a proximal portion of the proximal metatarsal to adjust a position alignment of the distal metatarsal portion and proximal metatarsal portion relative to each other in at transverse plane; and
guiding a screw through the proximal metatarsal portion and into the distal metatarsal portion through an opening of a screw targeting guide of the instrument along a screw insertion trajectory.

2. The method of claim 1, further comprising adjusting an angular orientation of the distal metatarsal portion relative to the proximal metatarsal portion in a frontal plane using a frontal-plane positioning device of the instrument.

3. The method of claim 2, wherein adjusting the position of the distal metatarsal portion relative to the proximal metatarsal portion in the frontal plane using the frontal-plane positioning device of the instrument comprises engaging a pin inserted into the distal bone portion and applying a force to the pin to move the distal bone portion in the frontal plane.

4. The method of claim 1, further comprising, prior to guiding the screw, identifying a target insertion location at the distal metatarsal portion by inserting at least one wire into the distal metatarsal portion.

5. The method of claim 4, wherein identifying the target insertion location at the distal metatarsal portion by inserting at least one wire into the distal metatarsal portion comprises identifying the target insertion location at the distal metatarsal portion by inserting a plurality of different length wires into the distal metatarsal portion.

6. The method of claim 4, further comprising selecting the screw insertion trajectory to intersect the target insertion location from one of a plurality of discrete different target insertion location options that the screw targeting guide is configured to target.

7. The method of claim 4, further comprising, after selecting the screw insertion trajectory to intersect the target insertion location, adjusting an initial insertion location of the screw insertion trajectory relative to a medial side of the proximal metatarsal portion.

8. The method of claim 1, wherein the opening of the screw targeting guide defining the screw insertion trajectory for guiding the screw comprises:
a first opening defining a first screw insertion trajectory for guiding a first screw; and
a second opening defining a second screw insertion trajectory for guiding a second screw;
wherein the second screw insertion trajectory is parallel to the first screw insertion trajectory.

9. The method of claim 1, comprising:
inserting an intramedullary insertion body of the instrument into a medullary canal of the proximal bone portion; and
positioning a proximal contact surface of the instrument against the proximal bone portion;
wherein applying the medial-directed pulling force to the distal end of the proximal metatarsal portion and applying the lateral-directed pushing force to the proximal portion of the proximal metatarsal comprises moving the intramedullary insertion body medially to apply the medial-directed pulling force and moving the proximal contact surface laterally to apply the lateral-directed pushing force.

10. The method of claim 9, further comprising positioning a distal contact surface of the instrument against the distal bone portion prior to applying the medial-directed pulling force to the distal end of the proximal metatarsal portion and applying the lateral-directed pushing force to the proximal portion of the proximal metatarsal.

11. The method of claim 9, wherein proximal contact surface is defined by a cup comprising:
a first contact surface configured to extend at least partially over a dorsal-medial and/or dorsal surface of the proximal bone portion;
a second contact surface configured to extend at least partially over a plantar-medial and/or plantar surface of the proximal bone portion; and
a cutout between the first contact surface and the second contact surface through which an instrument and/or the screw is configured to be inserted.

12. The method of claim 9, further comprising:
engaging a first intramedullary insertion body translation mechanism to control movement of the intramedullary insertion body relative to the instrument in a medial-to-lateral direction; and/or
engaging a second intramedullary insertion body translation mechanism to control movement of the intramedullary insertion body relative to the instrument in a distal-to-proximal direction.

13. The method of claim 1, further comprising, prior to guiding the screw, guiding a drill through the opening along the screw insertion trajectory.

14. The method of claim 13, wherein guiding the drill through the opening along the screw insertion trajectory comprises advancing the drill through the proximal metatarsal portion and into the distal metatarsal bone portion.

15. The method of claim 13, wherein guiding the drill through the opening along the screw insertion trajectory comprises advancing the drill through a medial cortical wall of the proximal metatarsal portion and up to but not through a lateral cortical wall of the proximal metatarsal bone portion.

16. The method of claim 15, further comprising, after advancing the drill through the medial cortical wall of the proximal metatarsal portion and up to but not through the lateral cortical wall of the proximal metatarsal bone portion, withdrawing the drill and inserting a sleeve into the proximal metatarsal bone portion following an opening formed by the drill.

17. The method of claim 16, further comprising advancing a wire through a cannulation of the sleeve, through the lateral cortical wall of the proximal metatarsal bone portion, and into the distal metatarsal bone portion.

18. The method of claim 17, further comprising withdrawing the sleeve over the wire and guiding the screw over the wire until the screw is positioned in the proximal metatarsal bone portion and the distal metatarsal bone portion.

19. The method of claim 1, further comprising positioning a support surface of a plantar support mechanism associated with the instrument plantary under the distal metatarsal portion.

20. The method of claim 19, further comprising actuating an adjuster of the plantar support mechanism to advance the support surface dorsally into contact with the plantar side of the distal metatarsal portion to resist plantar translation of the distal metatarsal portion and/or to adjust a sagittal-plane position of the distal metatarsal portion.
